(19) United States
(12) Patent Application Publication
(10) Pub. No.: US 2012/0208827 A1
(43) Pub. Date: Aug. 16, 2012

(54) BENZIMIDAZOLES AS FATTY ACID SYNTHASE INHIBITORS

(75) Inventors: Steven Dock, Research Triangle Park, NC (US); Jason Hallman, Cary, NC (US); Christopher Laudeman, Durham, NC (US); Ronggang Liu, Berwyn, PA (US); Aaron Miller, Research Triangle Park, NC (US); Michael Lee Moore, Collegeville, PA (US); David Musso, Raleigh, NC (US); Cynthia Parrish, Collegeville, PA (US)

(73) Assignee: GlaxoSmithKline LLC

(21) Appl. No.: 13/504,547

(22) PCT Filed: Oct. 27, 2010
(86) PCT No.: PCT/US10/54230

§ 371 Date: Apr. 27, 2012

Related U.S. Application Data
(60) Provisional application No. 61/255,230, filed on Oct. 27, 2009.
(60) Provisional application No. 61/256,096, filed on Oct. 29, 2009.

(51) Int. Cl. A61K 31/496; A61K 31/437; A61K 31/4725; A61K 31/428; A61K 31/423; A61K 31/4184; C07D 471/04; C07D 401/14; C07D 417/14; C07D 413/14; C07D 403/14; C07D 405/14; A61P 35/00; C07D 403/06; A61K 31/517

(52) U.S. Cl. 514/254.06; 514/266.23; 514/300; 514/303; 514/309; 514/367; 514/375; 514/394; 544/284; 544/370; 546/113; 546/119; 546/121; 546/141; 548/159; 548/217; 548/305.1

(57) ABSTRACT

This invention relates to the use of benzimidazole derivatives for the modulation, notably the inhibition of the activity or function of fatty acid synthase (FAS). Suitably, the present invention relates to the use of benzimidazoles in the treatment of cancer.

This application claims the benefit of U.S. provisional application No. 61/256,096, filed 29 Oct. 2009 and U.S. provisional application No. 61/255,230, filed 27 Oct. 2009, both which are incorporated herein in their entirety.

### FIELD OF INVENTION

This invention relates to novel benzimidazoles which are inhibitors of fatty acid synthase (FAS), to pharmaceutical compositions containing them, to processes for their preparation, and to their use in therapy for the treatment of cancers.

### BACKGROUND

Fatty acids have an essential role in a variety of cellular processes including building blocks for membranes, anchors for targeting membrane proteins, precursors in the synthesis of lipid second messengers and as a medium to store energy, Menendez J S and Lupu R, Fatty acid synthase and the lipogenic phenotype in cancer pathogenesis, Nature Reviews Cancer, 7: 763-777 (2007). Fatty acids can either be obtained from the diet or can be synthesized de novo from carbohydrate precursors. The biosynthesis of the latter is catalyzed by the muliti-functional homodimeric FAS. FAS synthesizes long chain fatty acids by using acetyl-CoA as a primer and Malonyl Co-A as a 2 carbon donor, and NADPH as a reducing equivalents (Wakil S J, Lipids, Structure and function of animal fatty acid synthase, 39: 1045-1053 (2004), Asturias F J et al., Structure and molecular organization of mammalian fatty acid synthase, Nature Struct. Mol. Biol. 12:225-232 (2005), Maier T, et al., Architecture of Mammalian Fatty Acid Synthase at 4.5 Å Resolution, Science 311:1258-1262 (2006).

De novo fatty acid synthesis is active during embryogenesis and in fetal lungs where fatty acids are used for the production of lung surfactant. In adults, most normal human tissues preferentially acquire fatty acids from the diet. Therefore the level of de novo lipogensis and expression of liopogenic enzymes is low, Weiss L, et al., Fatty-acid biosynthesis in man, a pathway of minor importance. Purification, optimal assay conditions, and organ distribution of fatty-acid synthase. Biological Chemistry Hoppe-Seyler 367(9):905-912 (1986). In contrast, many tumors have high rates of de novo fatty acid synthesis Medes G, et al., Metabolism of Neoplastic Tissue. IV. A Study of Lipid Synthesis in Neoplastic Tissue Slices in Vitro, Can Res, 13:27-29, (1953). FAS has now been shown to be overexpressed in numerous cancer types including prostate, ovary, colon, endometrium lung, bladder, stomach and kidney Kuhajda F P, Fatty-acid synthase and human cancer: new perspectives on its role in tumor biology, Nutrition; 16:202-208 (2000). This differential expression and function of FAS in tumors and normal cells provide an approach for cancer therapy with the potential of a substantial therapeutic window.

Pharmacological and small interference RNA mediated inhibition of FAS has demonstrated a preferential inhibition of cancer cell proliferation. Additionally these inhibitors induce apoptosis in cancers cells in vitro and retard growth in human tumors in murine xenograft models in vivo, Menendez J S and Lupu R, Nature Reviews Cancer, 7: 763-777 (2007). Based upon these findings, FAS is considered a major potential target of antineoplastic intervention.

### SUMMARY OF THE INVENTION

This invention relates to compound of the Formula (I), as shown below

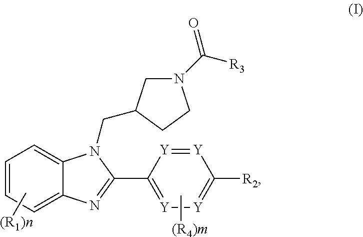

wherein,
each R₁ is independently selected from the group consisting of: halogen, C1-6alkyl, alkoxy, hydroxyl, amino, substituted amino, alkylsulfonyl, C4-7heterocycloalkyl, cyano, and —C(O)NRₐR_{b},
in which Rₐ and R_{b} are hydrogen, C1-6alkyl, C3-7cycloalkyl, or together Rₐ and R_{b} form a C4-7heterocycloalkyl;
R₂ is selected from the group consisting of: optionally substituted aryl and heteroaryl, in which adjacent substituents together may form an additional five or six membered ring which contains 0-2 hetero atoms;
R₃ is selected from the group consisting of: amino, alkylamino, dialkylamino, —OC1-6alkyl, C1-6alkyl and C3-7cycloalkyl;
R₄ is selected from the group consisting of: C1-6alkyl, alkoxy, hydroxyl and halogen;

### Y is C or N; and

n is 0-4;
m is 0-4;
or a pharmaceutically acceptable salt thereof;
with the proviso that at least two Y's are C.

This invention also relates to pharmaceutical compositions, which comprise compounds of Formula (I) and pharmaceutically acceptable carriers.

This invention also relates to methods of treating cancer which comprise administering an effective amount of a compound of Formula (I) to a human in need thereof.

This invention also relates to methods of treating cancer which comprise co-administering an compound of Formula (I) and a second compound to a human in need thereof.

### DETAILED DESCRIPTION OF THE INVENTION

This invention also relates to compound of the Formula (I)(A), as shown below

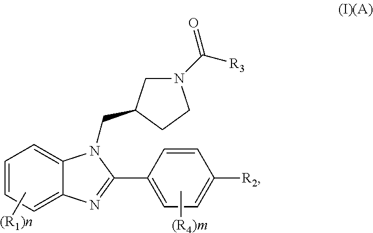

wherein,
each R₁ is independently selected from the group consisting of: C1-6alkyl, alkoxy, cyano, halogen, and —C(O)NRₐR_{b}, in which Rₐ and R_{b} are hydrogen, C1-6alkyl, C3-7cycloalkyl, or together Rₐ and R_{b} form a C4-7heterocycloalkyl;
R₂ is selected from the group consisting of: optionally substituted aryl and heteroaryl, in which adjacent substituents together may form an additional five or six membered ring which contains 0-2 hetero atoms;
R₃ is selected from the group consisting of: amino, alkylamino, dialkylamino, —OC1-6alkyl,
C1-6alkyl and C3-7cycloalkyl;

R₄ is selected from the group consisting of: C1-6alkyl, alkoxy, hydroxyl and halogen; and

n is 0-4
m is 0-4;
or a pharmaceutically acceptable salt thereof.

This invention also relates to compounds of Formula (I)(A), wherein R₃ is cyclopropyl.

This invention also relates to compounds of Formula (I)(A), wherein n is 0-2 and m is 0.

This invention also relates to compounds of Formula (I)(A), wherein n is 1 and m is 0.

This invention also relates to compounds of Formula (I)(A), wherein R₁ is halogen, cyano, alkoxy, C1-3alkyl, or —C(O)NRₐR_{b} as defined above.

This invention also relates to compounds of Formula (I)(A), wherein R₂ is heteroaryl.

This invention also relates to compounds of Formula (I)(A), wherein R₂ is aryl.

This invention also relates to compounds of Formula (I)(A), wherein R₂ is an aryl or heteroaryl selected from the group consisting of: indole, phenyl, indazole, benzofuranyl, wherein said aryl or heteroaryl may be substituted by one to three groups selected from: alkyl, halogen, hydroxyl, —SO₂Me and alkoxy.

This invention also relates to compounds exemplified in the Experimental section. This invention also relates to the following compounds:

### Example 1

1-{[(3S)-1-(Cyclopropylcarbonyl)-3-pyrrolidinyl]methyl}-2-[4-(1H-indol-5-yl)phenyl]-1H-benzimidazole,
1-{[(3S)-1-(Cyclopropylcarbonyl)-3-pyrrolidinyl]methyl}-2-[4-(1H-indol-6-yl)phenyl]-1H-benzimidazole,
2-[4-(1-Benzofuran-5-yl)phenyl]-1-{[(3S)-1-(cyclopropylcarbonyl)-3-pyrrolidinyl]methyl}-1H-benzimidazole,
6-[4-(1-{[(3S)-1-(Cyclopropylcarbonyl)-3-pyrrolidinyl]methyl}-1H-benzimidazol-2-yl)phenyl]-1,3-benzothiazole,
1-{[(3S)-1-(Cyclopropylcarbonyl)-3-pyrrolidinyl]methyl}-2-(4′-fluoro-4-biphenylyl)-4-(methyloxy)-1H-benzimidazole
1-{[(3S)-1-(Cyclopropylcarbonyl)-3-pyrrolidinyl]methyl}-4-(methyloxy)-2-[4′-(methyloxy)-4-biphenylyl]-1H-benzimidazole
1-{[(3S)-1-(Cyclopropylcarbonyl)-3-pyrrolidinyl]methyl}-2-[4-(1H-indol-5-yl)phenyl]-4-(methyloxy)-1H-benzimidazole
1-{[(3S)-1-(Cyclopropylcarbonyl)-3-pyrrolidinyl]methyl}-2-[4-(1H-indol-6-yl)phenyl]-4-(methyloxy)-1H-benzimidazole
5-{4-[1-{[(3S)-1-(Cyclopropylcarbonyl)-3-pyrrolidinyl]methyl}-4-(methyloxy)-1H-benzimidazol-2-yl]phenyl}-1H-indazole
6-{4-[1-{[(3S)-1-(Cyclopropylcarbonyl)-3-pyrrolidinyl]methyl}-4-(methyloxy)-1H-benzimidazol-2-yl]phenyl}-1H-indazole
2-(4-Biphenylyl)-1-{[(3S)-1-(cyclopropylcarbonyl)-3-pyrrolidinyl]methyl}-4-(methyloxy)-1H-benzimidazole
2-[4-(1-Benzofuran-5-yl)phenyl]-1-{[(3S)-1-(cyclopropylcarbonyl)-3-pyrrolidinyl]methyl}-4-(methyloxy)-1H-benzimidazole
1-{[(3S)-1-(Cyclopropylcarbonyl)-3-pyrrolidinyl]methyl}-2-(4′-fluoro-4-biphenylyl)-4-methyl-1H-benzimidazole
1-{[(3S)-1-(Cyclopropylcarbonyl)-3-pyrrolidinyl]methyl}-4-methyl-2-[4′-(methyloxy)-4-biphenylyl]-1H-benzimidazole
2-[4-(1-Benzofuran-5-yl)phenyl]-1-{[(3S)-1-(cyclopropylcarbonyl)-3-pyrrolidinyl]methyl}-4-methyl-1H-benzimidazole
1-{[(3S)-1-(Cyclopropylcarbonyl)-3-pyrrolidinyl]methyl}-2-[4-(1H-indol-5-yl)phenyl]-4-methyl-1H-benzimidazole
1-{[(3S)-1-(Cyclopropylcarbonyl)-3-pyrrolidinyl]methyl}-2-[4-(1H-indol-6-yl)phenyl]-4-methyl-1H-benzimidazole
5-[4-(1-{[(3S)-1-(Cyclopropylcarbonyl)-3-pyrrolidinyl]methyl}-4-methyl-1H-benzimidazol-2-yl)phenyl]-1H-indazole
6-[4-(1-{[(3S)-1-(Cyclopropylcarbonyl)-3-pyrrolidinyl]methyl}-4-methyl-1H-benzimidazol-2-yl)phenyl]-1H-indazole
2-(4-Biphenylyl)-1-{[(3S)-1-(cyclopropylcarbonyl)-3-pyrrolidinyl]methyl}-4-methyl-1H-benzimidazole
1-{[(3S)-1-(Cyclopropylcarbonyl)-3-pyrrolidinyl]methyl}-2-(4′-fluoro-4-biphenylyl)-4-(trifluoromethyl)-1H-benzimidazole
1-{[(3S)-1-(Cyclopropylcarbonyl)-3-pyrrolidinyl]methyl}-2-[4′-(methyloxy)-4-biphenylyl]-4-(trifluoromethyl)-1H-benzimidazole
1-{[(3S)-1-(Cyclopropylcarbonyl)-3-pyrrolidinyl]methyl}-2-[4-(1H-indol-5-yl)phenyl]-4-(trifluoromethyl)-1H-benzimidazole
1-{[(3S)-1-(Cyclopropylcarbonyl)-3-pyrrolidinyl]methyl}-2-[4-(1H-indol-6-yl)phenyl]-4-(trifluoromethyl)-1H-benzimidazole
5-{4-[1-{[(3S)-1-(Cyclopropylcarbonyl)-3-pyrrolidinyl]methyl}-4-(trifluoromethyl)-1H-benzimidazol-2-yl]phenyl}-1H-indazole
6-{4-[1-{[(3S)-1-(Cyclopropylcarbonyl)-3-pyrrolidinyl]methyl}-4-(trifluoromethyl)-1H-benzimidazol-2-yl]phenyl}-1H-indazole
2-[4-(1-Benzofuran-5-yl)phenyl]-1-{[(3S)-1-(cyclopropylcarbonyl)-3-pyrrolidinyl]methyl}-4-(trifluoromethyl)-1H-benzimidazole
2-(4-Biphenylyl)-1-{[(3S)-1-(cyclopropylcarbonyl)-3-pyrrolidinyl]methyl}-4-(trifluoromethyl)-1H-benzimidazole
4-Bromo-1-{[(3S)-1-(cyclopropylcarbonyl)-3-pyrrolidinyl]methyl}-2-(4′-fluoro-4-biphenylyl)-1H-benzimidazole
4-Bromo-1-{[(3S)-1-(cyclopropylcarbonyl)-3-pyrrolidinyl]methyl}-2-[4′-(methyloxy)-4-biphenylyl]-1H-benzimidazole
4-Bromo-1-{[(3S)-1-(cyclopropylcarbonyl)-3-pyrrolidinyl]methyl}-2-[4-(1H-indol-5-yl)phenyl]-1H-benzimidazole
5-[4-(4-Bromo-1-{[(3S)-1-(cyclopropylcarbonyl)-3-pyrrolidinyl]methyl}-1H-benzimidazol-2-yl)phenyl]-1H-indazole
2-(4-Biphenylyl)-4-bromo-1-{[(3S)-1-(cyclopropylcarbonyl)-3-pyrrolidinyl]methyl}-1H-benzimidazole
4-Bromo-1-{[(3S)-1-(cyclopropylcarbonyl)-3-pyrrolidinyl]methyl}-2-[4-(1H-indol-6-yl)phenyl]-1H-benzimidazole
6-[4-(4-Bromo-1-{[(3S)-1-(cyclopropylcarbonyl)-3-pyrrolidinyl]methyl}-1H-benzimidazol-2-yl)phenyl]-1H-indazole
2-[4-(1-Benzofuran-5-yl)phenyl]-4-bromo-1-{[(3S)-1-(cyclopropylcarbonyl)-3-pyrrolidinyl]methyl}-1H-benzimidazole
2-[4-(1-Benzofuran-5-yl)phenyl]-1-{[(3S)-1-(cyclopropylcarbonyl)-3-pyrrolidinyl]methyl}-5-(methyloxy)-1H-benzimidazole
1-{[(3S)-1-(Cyclopropylcarbonyl)-3-pyrrolidinyl]methyl}-2-[4-(1H-indol-5-yl)phenyl]-5-(methyloxy)-1H-benzimidazole
1-{[(3S)-1-(Cyclopropylcarbonyl)-3-pyrrolidinyl]methyl}-2-[4-(1H-indol-6-yl)phenyl]-5-(methyloxy)-1H-benzimidazole
5-{4-[1-{[(3S)-1-(Cyclopropylcarbonyl)-3-pyrrolidinyl]methyl}-5-(methyloxy)-1H-benzimidazol-2-yl]phenyl}-1H-indazole
6-{4-[1-{[(3S)-1-(Cyclopropylcarbonyl)-3-pyrrolidinyl]methyl}-5-(methyloxy)-1H-benzimidazol-2-yl]phenyl}-1H-indazole
2-(4-Biphenylyl)-1-{[(3S)-1-(cyclopropylcarbonyl)-3-pyrrolidinyl]methyl}-5-(methyloxy)-1H-benzimidazole
1-{[(3S)-1-(Cyclopropylcarbonyl)-3-pyrrolidinyl]methyl}-2-(4′-fluoro-4-biphenylyl)-5-(methyloxy)-1H-benzimidazole
1-{[(3S)-1-(Cyclopropylcarbonyl)-3-pyrrolidinyl]methyl}-5-(methyloxy)-2-[4′-(methyloxy)-4-biphenylyl]-1H-benzimidazole
1-{[(3S)-1-(Cyclopropylcarbonyl)-3-pyrrolidinyl]methyl}-2-(4′-fluoro-4-biphenylyl)-5-methyl-1H-benzimidazole
1-{[(3S)-1-(Cyclopropylcarbonyl)-3-pyrrolidinyl]methyl}-5-methyl-2-[4′-(methyloxy)-4-biphenylyl]-1H-benzimidazole
1-{[(3S)-1-(Cyclopropylcarbonyl)-3-pyrrolidinyl]methyl}-2-[4-(1H-indol-5-yl)phenyl]-5-methyl-1H-benzimidazole
1-{[(3S)-1-(Cyclopropylcarbonyl)-3-pyrrolidinyl]methyl}-2-[4-(1H-indol-6-yl)phenyl]-5-methyl-1H-benzimidazole
5-[4-(1-{[(3S)-1-(Cyclopropylcarbonyl)-3-pyrrolidinyl]methyl}-5-methyl-1H-benzimidazol-2-yl)phenyl]-1H-indazole
6-[4-(1-{[(3S)-1-(Cyclopropylcarbonyl)-3-pyrrolidinyl]methyl}-5-methyl-1H-benzimidazol-2-yl)phenyl]-1H-indazole
2-(4-Biphenylyl)-1-{[(3S)-1-(cyclopropylcarbonyl)-3-pyrrolidinyl]methyl}-5-methyl-1H-benzimidazole
2-[4-(1-Benzofuran-5-yl)phenyl]-1-{[(3S)-1-(cyclopropylcarbonyl)-3-pyrrolidinyl]methyl}-5-methyl-1H-benzimidazole
5-{4-[1-{[(3S)-1-(Cyclopropylcarbonyl)-3-pyrrolidinyl]methyl}-5-(trifluoromethyl)-1H-benzimidazol-2-yl]phenyl}-1H-indazole
1-{[(3S)-1-(Cyclopropylcarbonyl)-3-pyrrolidinyl]methyl}-2-[4-(1H-indol-6-yl)phenyl]-5-(trifluoromethyl)-1H-benzimidazole
6-{4-[1-{[(3S)-1-(Cyclopropylcarbonyl)-3-pyrrolidinyl]methyl}-5-(trifluoromethyl)-1H-benzimidazol-2-yl]phenyl}-1H-indazole
2-(4-biphenylyl)-1-{[(3S)-1-(Cyclopropylcarbonyl)-3-pyrrolidinyl]methyl}-5-(trifluoromethyl)-1H-benzimidazole
2-[4-(1-Benzofuran-5-yl)phenyl]-1-{[(3S)-1-(cyclopropylcarbonyl)-3-pyrrolidinyl]methyl}-5-(trifluoromethyl)-1H-benzimidazole
4′-[1-{[(3S)-1-(Cyclopropylcarbonyl)-3-pyrrolidinyl]methyl}-5-(trifluoromethyl)-1H-benzimidazol-2-yl]-3-biphenylol
1-{[(3S)-1-(Cyclopropylcarbonyl)-3-pyrrolidinyl]methyl}-2-(4′-fluoro-4-biphenylyl)-5-(trifluoromethyl)-1H-benzimidazole
1-{[(3S)-1-(Cyclopropylcarbonyl)-3-pyrrolidinyl]methyl}-2-[4′-(methyloxy)-4-biphenylyl]-5-(trifluoromethyl)-1H-benzimidazole
2-(3′-Chloro-4-biphenylyl)-1-{[(3S)-1-(cyclopropylcarbonyl)-3-pyrrolidinyl]methyl}-5-(trifluoromethyl)-1H-benzimidazole
1-{[(3S)-1-(Cyclopropylcarbonyl)-3-pyrrolidinyl]methyl}-2-[4-(1H-indol-5-yl)phenyl]-5-(trifluoromethyl)-1H-benzimidazole
5-Bromo-1-{[(3S)-1-(cyclopropylcarbonyl)-3-pyrrolidinyl]methyl}-6-fluoro-2-[4-(1H-indol-5-yl)phenyl]-1H-benzimidazole
2-(4-Biphenylyl)-5-bromo-1-{[(3S)-1-(cyclopropylcarbonyl)-3-pyrrolidinyl]methyl}-6-fluoro-1H-benzimidazole
5-Bromo-1-{[(3S)-1-(cyclopropylcarbonyl)-3-pyrrolidinyl]methyl}-6-fluoro-2-[4′-(methyloxy)-4-biphenylyl]-1H-benzimidazole
1-{[(3S)-1-(Cyclopropylcarbonyl)-3-pyrrolidinyl]methyl}-2-[4-(1H-indol-6-yl)phenyl]-6-(methyloxy)-1H-benzimidazole
4′-[1-{[(3S)-1-(cyclopropylcarbonyl)-3-pyrrolidinyl]methyl}-6-(methyloxy)-1H-benzimidazol-2-yl]-3-biphenylol
1-{[(3S)-1-(Cyclopropylcarbonyl)-3-pyrrolidinyl]methyl}-2-(4′-fluoro-4-biphenylyl)-6-(methyloxy)-1H-benzimidazole
1-{[(3S)-1-(Cyclopropylcarbonyl)-3-pyrrolidinyl]methyl}-6-(methyloxy)-2-[4′-(methyloxy)-4-biphenylyl]-1H-benzimidazole
1-{[(3S)-1-(Cyclopropylcarbonyl)-3-pyrrolidinyl]methyl}-2-[4-(1H-indol-5-yl)phenyl]-6-(methyloxy)-1H-benzimidazole
5-{4-[1-{[(3S)-1-(Cyclopropylcarbonyl)-3-pyrrolidinyl]methyl}-6-(methyloxy)-1H-benzimidazol-2-yl]phenyl}-1H-indazole
6-{4-[1-{[(3S)-1-(Cyclopropylcarbonyl)-3-pyrrolidinyl]methyl}-6-(methyloxy)-1H-benzimidazol-2-yl]phenyl}-1H-indazole
2-(4-Biphenylyl)-1-{[(3S)-1-(cyclopropylcarbonyl)-3-pyrrolidinyl]methyl}-6-(methyloxy)-1H-benzimidazole
2-[4-(1-Benzofuran-5-yl)phenyl]-1-{[(3S)-1-(cyclopropylcarbonyl)-3-pyrrolidinyl]methyl}-6-(methyloxy)-1H-benzimidazole
4′-(1-{[(3S)-1-(Cyclopropylcarbonyl)-3-pyrrolidinyl]methyl}-6-methyl-1H-benzimidazol-2-yl)-3-biphenylol
1-{[(3S)-1-(Cyclopropylcarbonyl)-3-pyrrolidinyl]methyl}-2-(4′-fluoro-4-biphenylyl)-6-methyl-1H-benzimidazole
1-{[(3S)-1-(Cyclopropylcarbonyl)-3-pyrrolidinyl]methyl}-6-methyl-2-[4′-(methyloxy)-4-biphenylyl]-1H-benzimidazole
1-{[(3S)-1-(Cyclopropylcarbonyl)-3-pyrrolidinyl]methyl}-2-[4-(1H-indol-5-yl)phenyl]-6-methyl-1H-benzimidazole
1-{[(3S)-1-(Cyclopropylcarbonyl)-3-pyrrolidinyl]methyl}-2-[4-(1H-indol-6-yl)phenyl]-6-methyl-1H-benzimidazole
6-[4-(1-{[(3S)-1-(Cyclopropylcarbonyl)-3-pyrrolidinyl]methyl}-6-methyl-1H-benzimidazol-2-yl)phenyl]-1H-indazole
5-[4-(1-{[(3S)-1-(Cyclopropylcarbonyl)-3-pyrrolidinyl]methyl}-6-methyl-1H-benzimidazol-2-yl)phenyl]-1H-indazole
2-(4-Biphenylyl)-1-{[(3S)-1-(cyclopropylcarbonyl)-3-pyrrolidinyl]methyl}-6-methyl-1H-benzimidazole
2-[4-(1-Benzofuran-5-yl)phenyl]-1-{[(3S)-1-(cyclopropylcarbonyl)-3-pyrrolidinyl]methyl}-6-(methyl)-1H-benzimidazole
4′-[1-{[(3S)-1-(Cyclopropylcarbonyl)-3-pyrrolidinyl]methyl}-6-(trifluoromethyl)-1H-benzimidazol-2-yl]-3-biphenylol
1-{[(3S)-1-(Cyclopropylcarbonyl)-3-pyrrolidinyl]methyl}-2-(4′-fluoro-4-biphenylyl)-6-(trifluoromethyl)-1H-benzimidazole
1-{[(3S)-1-(Cyclopropylcarbonyl)-3-pyrrolidinyl]methyl}-2-[4′-(methyloxy)-4-biphenylyl]-6-(trifluoromethyl)-1H-benzimidazole
1-{[(3S)-1-(Cyclopropylcarbonyl)-3-pyrrolidinyl]methyl}-2-[4-(1H-indol-5-yl)phenyl]-6-(trifluoromethyl)-1H-benzimidazole
1-{[(3S)-1-(Cyclopropylcarbonyl)-3-pyrrolidinyl]methyl}-2-[4-(1H-indol-6-yl)phenyl]-6-(trifluoromethyl)-1H-benzimidazole
5-{4-[1-{[(3S)-1-(cyclopropylcarbonyl)-3-pyrrolidinyl]methyl}-6-(trifluoromethyl)-1H-benzimidazol-2-yl]phenyl}-1H-indazole
6-{4-[1-{[(3S)-1-(cyclopropylcarbonyl)-3-pyrrolidinyl]methyl}-6-(trifluoromethyl)-1H-benzimidazol-2-yl]phenyl}-1H-indazole
2-(4-biphenylyl)-1-{[(3S)-1-(cyclopropylcarbonyl)-3-pyrrolidinyl]methyl}-6-(trifluoromethyl)-1H-benzimidazole
2-[4-(1-benzofuran-5-yl)phenyl]-1-{[(3S)-1-(cyclopropylcarbonyl)-3-pyrrolidinyl]methyl}-6-(trifluoromethyl)-1H-benzimidazole
2-(4-Biphenylyl)-6-bromo-1-{[(3S)-1-(cyclopropylcarbonyl)-3-pyrrolidinyl]methyl}-1H-benzimidazole
4′-(B-bromo-1-{[(3S)-1-(cyclopropylcarbonyl)-3-pyrrolidinyl]methyl}-1H-benzimidazol-2-yl)-3-biphenylol
6-Bromo-1-{[(3S)-1-(cyclopropylcarbonyl)-3-pyrrolidinyl]methyl}-2-(4′-fluoro-4-biphenylyl)-1H-benzimidazole
6-Bromo-1-{[(3S)-1-(cyclopropylcarbonyl)-3-pyrrolidinyl]methyl}-2-[4′-(methyloxy)-4-biphenylyl]-1H-benzimidazole
6-Bromo-1-{[(3S)-1-(cyclopropylcarbonyl)-3-pyrrolidinyl]methyl}-2-[4-(1H-indol-5-yl)phenyl]-1H-benzimidazole
6-Bromo-1-{[(3S)-1-(cyclopropylcarbonyl)-3-pyrrolidinyl]methyl}-2-[4-(1H-indol-6-yl)phenyl]-1H-benzimidazole
5-[4-(6-Bromo-1-{[(3S)-1-(cyclopropylcarbonyl)-3-pyrrolidinyl]methyl}-1H-benzimidazol-2-yl)phenyl]-1H-indazole
6-[4-(B-bromo-1-{[(3S)-1-(cyclopropylcarbonyl)-3-pyrrolidinyl]methyl}-1H-benzimidazol-2-yl)phenyl]-1H-indazole
2-[4-(1-Benzofuran-5-yl)phenyl]-6-bromo-1-{[(3S)-1-(cyclopropylcarbonyl)-3-pyrrolidinyl]methyl}-1H-benzimidazole
2-(4-Biphenylyl)-1-{[(3S)-1-(cyclopropylcarbonyl)-3-pyrrolidinyl]methyl}-N-methyl-1H-benzimidazole-6-carboxamide
1-{[(3S)-1-(cyclopropylcarbonyl)-3-pyrrolidinyl]methyl}-2-(4′-fluoro-4-biphenylyl)-N-methyl-1H-benzimidazole-6-carboxamide
1-{[(3S)-1-(Cyclopropylcarbonyl)-3-pyrrolidinyl]methyl}-N-methyl-2-[4′-(methyloxy)-4-biphenylyl]-1H-benzimidazole-6-carboxamide
1-{[(3S)-1-(Cyclopropylcarbonyl)-3-pyrrolidinyl]methyl}-2-(4′-fluoro-4-biphenylyl)-6-[(4-methyl-1-piperazinyl)carbonyl]-1H-benzimidazole
1-{[(3S)-1-(Cyclopropylcarbonyl)-3-pyrrolidinyl]methyl}-2-[4-(1H-indol-6-yl)phenyl]-6-[(4-methyl-1-piperazinyl)carbonyl]-1H-benzimidazole
1-{[(3S)-1-(Cyclopropylcarbonyl)-3-pyrrolidinyl]methyl}-2-[4-(1H-indol-5-yl)phenyl]-6-[(4-methyl-1-piperazinyl)carbonyl]-1H-benzimidazole
1-{[(3S)-1-(Cyclopropylcarbonyl)-3-pyrrolidinyl]methyl}-7-methyl-2-[4′-(methyloxy)-4-biphenylyl]-1H-benzimidazole
1-{[(3S)-1-(Cyclopropylcarbonyl)-3-pyrrolidinyl]methyl}-2-[4-(1H-indol-5-yl)phenyl]-7-methyl-1H-benzimidazole
1-{[(3S)-1-(Cyclopropylcarbonyl)-3-pyrrolidinyl]methyl}-2-[4-(1H-indol-6-yl)phenyl]-7-methyl-1H-benzimidazole
5-[4-(1-{[(3S)-1-(Cyclopropylcarbonyl)-3-pyrrolidinyl]methyl}-7-methyl-1H-benzimidazol-2-yl)phenyl]-1H-indazole
2-(4-Biphenylyl)-1-{[(3S)-1-(cyclopropylcarbonyl)-3-pyrrolidinyl]methyl}-7-methyl-1H-benzimidazole
4′-(1-{[(3S)-1-(Cyclopropylcarbonyl)-3-pyrrolidinyl]methyl}-7-methyl-1H-benzimidazol-2-yl)-3-biphenylol
1-{[(3S)-1-(Cyclopropylcarbonyl)-3-pyrrolidinyl]methyl}-2-(4′-fluoro-4-biphenylyl)-7-methyl-1H-benzimidazole
6-[4-(1-{[(3S)-1-(Cyclopropylcarbonyl)-3-pyrrolidinyl]methyl}-7-methyl-1H-benzimidazol-2-yl)phenyl]-1H-indazole
2-[4-(1-Benzofuran-5-yl)phenyl]-1-{[(3S)-1-(cyclopropylcarbonyl)-3-pyrrolidinyl]methyl}-7-methyl-1H-benzimidazole
1-{[(3S)-1-(Cyclopropylcarbonyl)-3-pyrrolidinyl]methyl}-2-(4′-fluoro-4-biphenylyl)-7-(trifluoromethyl)-1H-benzimidazole
1-{[(3S)-1-(Ccyclopropylcarbonyl)-3-pyrrolidinyl]methyl}-2-[4′-(methyloxy)-4-biphenylyl]-7-(trifluoromethyl)-1H-benzimidazole
2-[4-(1-Benzofuran-5-yl)phenyl]-1-{[(3S)-1-(cyclopropylcarbonyl)-3-pyrrolidinyl]methyl}-7-(trifluoromethyl)-1H-benzimidazole
1-{[(3S)-1-(Cyclopropylcarbonyl)-3-pyrrolidinyl]methyl}-2-[4-(1H-indol-5-yl)phenyl]-7-(trifluoromethyl)-1H-benzimidazole
5-{4-[1-{[(3S)-1-(Cyclopropylcarbonyl)-3-pyrrolidinyl]methyl}-7-(trifluoromethyl)-1H-benzimidazol-2-yl]phenyl}-1H-indazole
6-{4-[1-{[(3S)-1-(Cyclopropylcarbonyl)-3-pyrrolidinyl]methyl}-7-(trifluoromethyl)-1H-benzimidazol-2-yl]phenyl}-1H-indazole
1-{[(3S)-1-(Cyclopropylcarbonyl)-3-pyrrolidinyl]methyl}-2-[4-(1H-indol-6-yl)phenyl]-7-(trifluoromethyl)-1H-benzimidazole
2-(4-Biphenylyl)-7-bromo-1-{[(3S)-1-(cyclopropylcarbonyl)-3-pyrrolidinyl]methyl}-1H-benzimidazole
4′-(7-Bromo-1-{[(3S)-1-(cyclopropylcarbonyl)-3-pyrrolidinyl]methyl}-1H-benzimidazol-2-yl)-3-biphenylol
7-Bromo-1-{[(3S)-1-(cyclopropylcarbonyl)-3-pyrrolidinyl]methyl}-2-(4′-fluoro-4-biphenylyl)-1H-benzimidazole
7-Bromo-1-{[(3S)-1-(cyclopropylcarbonyl)-3-pyrrolidinyl]methyl}-2-[4′-(methyloxy)-4-biphenylyl]-1H-benzimidazole
7-Bromo-1-{[(3S)-1-(cyclopropylcarbonyl)-3-pyrrolidinyl]methyl}-2-[4-(1H-indol-5-yl)phenyl]-1H-benzimidazole
5-[4-(7-Bromo-1-{[(3S)-1-(cyclopropylcarbonyl)-3-pyrrolidinyl]methyl}-1H-benzimidazol-2-yl)phenyl]-1H-indazole
7-Bromo-1-{[(3S)-1-(cyclopropylcarbonyl)-3-pyrrolidinyl]methyl}-2-[4-(1H-indol-6-yl)phenyl]-1H-benzimidazole
6-[4-(7-Bromo-1-{[(3S)-1-(cyclopropylcarbonyl)-3-pyrrolidinyl]methyl}-1H-benzimidazol-2-yl)phenyl]-1H-indazole
2-[4-(1-Benzofuran-5-yl)phenyl]-7-bromo-1-{[(3S)-1-(cyclopropylcarbonyl)-3-pyrrolidinyl]methyl}-1H-benzimidazole
1-{[(3S)-1-(cyclopropylcarbonyl)-3-pyrrolidinyl]methyl}-2-(3′-hydroxy-4-biphenylyl)-N-methyl-1H-benzimidazole-7-carboxamide
1-{[(3S)-1-(Cyclopropylcarbonyl)-3-pyrrolidinyl]methyl}-2-(4′-fluoro-4-biphenylyl)-N-methyl-1H-benzimidazole-7-carboxamide
1-{[(3S)-1-(Cyclopropylcarbonyl)-3-pyrrolidinyl]methyl}-2-[4-(1H-indol-5-yl)phenyl]-N-methyl-1H-benzimidazole-7-carboxamide
1-{[(3S)-1-(Cyclopropylcarbonyl)-3-pyrrolidinyl]methyl}-2-[4-(1H-indol-6-yl)phenyl]-N-methyl-1H-benzimidazole-7-carboxamide
1-{[(3S)-1-(Cyclopropylcarbonyl)-3-pyrrolidinyl]methyl}-2-[4-(1H-indazol-5-yl)phenyl]-N-methyl-1H-benzimidazole-7-carboxamide
1-{[(3S)-1-(Cyclopropylcarbonyl)-3-pyrrolidinyl]methyl}-2-[4-(1H-indazol-6-yl)phenyl]-N-methyl-1H-benzimidazole-7-carboxamide
2-(4-Diphenylyl)-1-{[(3S)-1-(cyclopropylcarbonyl)-3-pyrrolidinyl]methyl}-N-methyl-1H-benzimidazole-7-carboxamide
1-{[(3S)-1-(Cyclopropylcarbonyl)-3-pyrrolidinyl]methyl}-N-methyl-2-[4′-(methyloxy)-4-biphenylyl]-1H-benzimidazole-7-carboxamide
2-[4-(1-Benzofuran-5-yl)phenyl]-1-{[(3S)-1-(cyclopropylcarbonyl)-3-pyrrolidinyl]methyl}-N-methyl-1H-benzimidazole-7-carboxamide
2-(4-Biphenylyl)-1-({(3RS)-1-[(dimethylamino)carbonyl]-3-pyrrolidinyl}methyl)-N-methyl-1H-benzimidazole-6-carboxamide
2-(4-Biphenylyl)-N-methyl-1-({(3RS)-1-[(3-methyl-5-isoxazolyl)carbonyl]-3-pyrrolidinyl}methyl)-1H-benzimidazole-6-carboxamide
1-{[(3S)-1-(Cyclopropylcarbonyl)-3-pyrrolidinyl]methyl}-2-[4-(1H-pyrrolo[3,2-b]pyridin-6-yl)phenyl]-5-(trifluoromethyl)-1H-benzimidazole
1-{[(3S)-1-(Cyclopropylcarbonyl)-3-pyrrolidinyl]methyl}-2-(4-imidazo[1,2-a]pyridin-7-ylphenyl)-5-(trifluoromethyl)-1H-benzimidazole
5-{4-[1-{[(3S)-1-(Cyclopropylcarbonyl)-3-pyrrolidinyl]methyl}-5-(trifluoromethyl)-1H-benzimidazol-2-yl]phenyl}-1H-pyrazolo[3,4-b]pyridine
5-{4-[1-{[(3S)-1-(Cyclopropylcarbonyl)-3-pyrrolidinyl]methyl}-5-(trifluoromethyl)-1H-benzimidazol-2-yl]phenyl}-1,3-benzoxazole
6-{4-[1-{[(3S)-1-(Cyclopropylcarbonyl)-3-pyrrolidinyl]methyl}-5-(trifluoromethyl)-1H-benzimidazol-2-yl]phenyl}-1,3-benzothiazole
1-{[(3S)-1-(Cyclopropylcarbonyl)-3-pyrrolidinyl]methyl}-2-[4-(1H-pyrrolo[2,3-b]pyridin-5-yl)phenyl]-5-(trifluoromethyl)-1H-benzimidazole
1-{[(3S)-1-(Cyclopropylcarbonyl)-3-pyrrolidinyl]methyl}-2-(4-imidazo[1,5-a]pyridin-5-ylphenyl)-5-(trifluoromethyl)-1H-benzimidazole
1-{[(3S)-1-(Cyclopropylcarbonyl)-3-pyrrolidinyl]methyl}-2-(4-imidazo[1,2-a]pyridin-5-ylphenyl)-5-(trifluoromethyl)-1H-benzimidazole
2-[4-(1-Benzofuran-6-yl)phenyl]-1-{[(3S)-1-(cyclopropylcarbonyl)-3-pyrrolidinyl]methyl}-5-(trifluoromethyl)-1H-benzimidazole
1-{[(3S)-1-(Cyclopropylcarbonyl)-3-pyrrolidinyl]methyl}-2-(4-imidazo[1,2-a]pyridin-3-ylphenyl)-5-(trifluoromethyl)-1H-benzimidazole
1-{[(3S)-1-(Cyclopropylcarbonyl)-3-pyrrolidinyl]methyl}-2-[3′-(methylsulfonyl)-4-biphenylyl]-5-(trifluoromethyl)-1H-benzimidazole
1-{[(3S)-1-(Cyclopropylcarbonyl)-3-pyrrolidinyl]methyl}-2-[4′-(methylsulfonyl)-4-biphenylyl]-5-(trifluoromethyl)-1H-benzimidazole
6-{4-[1-{[(3S)-1-(Cyclopropylcarbonyl)-3-pyrrolidinyl]methyl}-5-(trifluoromethyl)-1H-benzimidazol-2-yl]phenyl}-1,3-benzoxazole
5-{4-[1-{[(3S)-1-(Cyclopropylcarbonyl)-3-pyrrolidinyl]methyl}-5-(trifluoromethyl)-1H-benzimidazol-2-yl]phenyl}-1,3-dihydro-2H-indol-2-one
1-{[(3S)-1-(Cyclopropylcarbonyl)-3-pyrrolidinyl]methyl}-2-[4-(2,3-dihydro-1H-indol-5-yl)phenyl]-5-(trifluoromethyl)-1H-benzimidazole
1-{[(3S)-1-(Cyclopropylcarbonyl)-3-pyrrolidinyl]methyl}-2-[4-(1H-pyrrolo[2,3-b]pyridin-6-yl)phenyl]-5-(trifluoromethyl)-1H-benzimidazole
6-{4-[1-{[(3S)-1-(Cyclopropylcarbonyl)-3-pyrrolidinyl]methyl}-5-(trifluoromethyl)-1H-benzimidazol-2-yl]phenyl}-1H-pyrazolo[3,4-b]pyridine
2-[4-(1-Benzofuran-3-yl)phenyl]-1-{[(3S)-1-(cyclopropylcarbonyl)-3-pyrrolidinyl]methyl}-5-(trifluoromethyl)-1H-benzimidazole
4′-[1-{[(3S)-1-(Cyclopropylcarbonyl)-3-pyrrolidinyl]methyl}-5-(trifluoromethyl)-1H-benzimidazol-2-yl]-4-biphenylcarbonitrile
6-{4-[1-{[(3S)-1-(Cyclopropylcarbonyl)-3-pyrrolidinyl]methyl}-5-(trifluoromethyl)-1H-benzimidazol-2-yl]phenyl}quinazoline
1-{[(3S)-1-(Cyclopropylcarbonyl)-3-pyrrolidinyl]methyl}-2-[4-(1H-pyrrolo[3,2-c]pyridin-3-yl)phenyl]-5-(trifluoromethyl)-1H-benzimidazole
2-[4-(1H-Benzimidazol-5-yl)phenyl]-1-{[3S)-1-(cyclopropylcarbonyl)-3-pyrrolidinyl]methyl}-5-(trifluoromethyl)-1H-benzimidazole
6-{4-[1-{[(3S)-1-(Cyclopropylcarbonyl)-3-pyrrolidinyl]methyl}-5-(trifluoromethyl)-1H-benzimidazol-2-yl]phenyl}-1(2H)-isoquinolinone
1-{[(3S)-1-(Cyclopropylcarbonyl)-3-pyrrolidinyl]methyl}-2-[4-(2-methyl-1H-indol-5-yl)phenyl]-5-(trifluoromethyl)-1H-benzimidazole
2-[4-(1-Benzofuran-5-yl)phenyl]-1-{[(3S)-1-(cyclopropylcarbonyl)-3-pyrrolidinyl]methyl}-1H-benzimidazole-5-carbonitrile
1-{[(3S)-1-(Cyclopropylcarbonyl)-3-pyrrolidinyl]methyl}-2-[4-(1H-indol-5-yl)phenyl]-1H-benzimidazole-5-carbonitrile
2-[4-(1-Benzofuran-5-yl)phenyl]-1-{[(3S)-1-(cyclopropylcarbonyl)-3-pyrrolidinyl]methyl}-1H-benzimidazole-6-carbonitrile
1-{[(3S)-1-(Cyclopropylcarbonyl)-3-pyrrolidinyl]methyl}-2-[4-(1H-indol-5-yl)phenyl]-1H-benzimidazole-6-carbonitrile
1-{[(3S)-1-(Cyclopropylcarbonyl)-3-pyrrolidinyl]methyl}-2-[4-(1H-indol-6-yl)phenyl]-1H-benzimidazole-6-carbonitrile
2-[4-(1-Benzofuran-5-yl)phenyl]-1-{[(3S)-1-(cyclopropylcarbonyl)-3-pyrrolidinyl]methyl}-1H-benzimidazole-7-carbonitrile
1-{[(3S)-1-(Cyclopropylcarbonyl)-3-pyrrolidinyl]methyl}-2-[4-(1H-indol-6-yl)phenyl]-1H-benzimidazole-7-carbonitrile
1-{[(3S)-1-(cyclopropylcarbonyl)-3-pyrrolidinyl]methyl}-2-[4-(1H-indol-5-yl)phenyl]-1H-benzimidazole-7-carbonitrile
N′-[4′-(7-cyano-1-{[(3S)-1-(cyclopropylcarbonyl)-3-pyrrolidinyl]methyl}-1H-benzimidazol-2-yl)-3-biphenylyl]-N,N-dimethylsulfamide
2-[4-(1-Benzofuran-5-yl)phenyl]-1-{[(3S)-1-(cyclopropylcarbonyl)-3-pyrrolidinyl]methyl}-1H-benzimidazole-4-carbonitrile
1-{[(3S)-1-(Cyclopropylcarbonyl)-3-pyrrolidinyl]methyl}-2-[4-(1H-indol-5-yl)phenyl]-1H-benzimidazole-4-carbonitrile
1-{[(3S)-1-(cyclopropylcarbonyl)-3-pyrrolidinyl]methyl}-2-[4-(1H-indol-6-yl)phenyl]-1H-benzimidazole-4-carbonitrile
5-[4-(1-{[(3S)-1-(Cyclopropylcarbonyl)-3-pyrrolidinyl]methyl}-6-fluoro-1H-benzimidazol-2-yl)phenyl]-1H-indazole
1-{[(3S)-1-(Cyclopropylcarbonyl)-3-pyrrolidinyl]methyl}-6-fluoro-2-[4-(1H-indol-6-yl)phenyl]-1H-benzimidazole
6-[4-(1-{[(3S)-1-(Cyclopropylcarbonyl)-3-pyrrolidinyl]methyl}-6-fluoro-1H-benzimidazol-2-yl)phenyl]-1H-indazole
N-[4′-(1-{[(3S)-1-(cyclopropylcarbonyl)-3-pyrrolidinyl]methyl}-6-fluoro-1H-benzimidazol-2-yl)-3-biphenylyl]-N,N-dimethylsulfamide
1-{[(3S)-1-(Cyclopropylcarbonyl)-3-pyrrolidinyl]methyl}-6-fluoro-2-(4′-fluoro-4-biphenylyl)-1H-benzimidazole
1-{[(3S)-1-(Cyclopropylcarbonyl)-3-pyrrolidinyl]methyl}-6-fluoro-2-[4-(1H-indol-5-yl)phenyl]-1H-benzimidazole
2-[4-(1-Benzofuran-5-yl)phenyl]-1-{[(3S)-1-(cyclopropylcarbonyl)-3-pyrrolidinyl]methyl}-6-fluoro-1H-benzimidazole
2-[4-(1-Benzofuran-5-yl)phenyl]-1-{[(3S)-1-(cyclopropylcarbonyl)-3-pyrrolidinyl]methyl}-5-fluoro-1H-benzimidazole
1-{[(3S)-1-(Cyclopropylcarbonyl)-3-pyrrolidinyl]methyl}-5-fluoro-2-[4-(1H-indol-6-yl)phenyl]-1H-benzimidazole
1-{[(3S)-1-(Cyclopropylcarbonyl)-3-pyrrolidinyl]methyl}-5-fluoro-2-[4-(1H-indol-5-yl)phenyl]-1H-benzimidazole and
5-[4-(1-{[(3S)-1-(cyclopropylcarbonyl)-3-pyrrolidinyl]methyl}-5-fluoro-1H-benzimidazol-2-yl)phenyl]-1H-indazole

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. In general, the salts are formed from pharmaceutically acceptable inorganic and organic acids. More specific examples of suitable acid salts include maleic, hydrochloric, hydrobromic, sulphuric, phosphoric, nitric, perchloric, fumic, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methansulfonic (mesylate), naphthalene-2-sulfonic, benzenesulfonic, hydroxynaphthoic, hydroiodic, malic, teroic, tannic, and the like.

Other representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, oxalate, pamoate (embonate), palmitate, pantothenate, phosphate/diphosphate, polygalacturonate, salicylate, stearate, subacetate, succinate, sulfate, tannate, tartrate, teoclate, tosylate, triethiodide, and valerate salts.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

The compound of Formula (I) or a salt thereof may exist in stereoisomeric forms (e.g., it contains one or more asymmetric carbon atoms). The individual stereoisomers (enantiomers and diastereomers) and mixtures of these are included within the scope of the present invention. The invention also covers the individual isomers of the compound or salt represented by Formula (I) as mixtures with isomers thereof in which one or more chiral centers are inverted. Likewise, it is understood that a compound or salt of Formula (I) may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. Also included within the scope of the invention are individual isomers of the compound represented by Formula (I), as well as any wholly or partially equilibrated mixtures thereof. The present invention also includes the individual isomers of the compound or salt represented by the Formula (I) as well as mixtures with isomers thereof in which one or more chiral centers are inverted. It is to be understood that the present invention includes all combinations and subsets of the particular groups defined hereinabove.

### DEFINITIONS

Terms are used within their accepted meanings The following definitions are meant to clarify, but not limit, the terms defined.

As used herein, the term “alkyl” (or “alkylene”) refers to a straight or branched chain alkyl, preferably having from one to twelve carbon atoms, which may be unsubstituted or substituted, saturated or unsaturated with multiple degrees of substitution included within the present invention. Suitable substituents are selected from the group consisting of halogen, amino, substituted amino, cyano, hydroxyl, alkoxy, alkylthio, alkylsulfonyl, amidosulfonyl, oxazole and methylisoxazole. Examples of “alkyl” as used herein include methyl, ethyl, propyl, isopropyl, isobutyl, n-butyl, t-butyl, isopentyl, n-pentyl, and the like, as well as substituted versions thereof.

As used herein, the term “cycloalkyl” refers to an unsubstituted or substituted mono- or polycyclic non-aromatic saturated ring, which optionally includes an alkylene linker through which the cycloalkyl may be attached. Exemplary “cycloalkyl” groups include, but are not limited to, cyclopropyl, cyclobutyl, cyclopentyl, cyclohexyl, cycloheptyl, and the like, as well as unsubstituted and substituted versions thereof.

As used herein, the term “alkoxy” refers to the group —ORa, where Ra is C1-3alkyl or C3-7cycloalkyl as defined above.

As used herein, the term “substituted amino” is meant —NR′R″ wherein each R′ and R″ is independently selected from a group including hydrogen, C1-6alkyl, acyl, C3-C7cycloalkyl, wherein at least one of R′ and R″ is not hydrogen. Examples of substituted amino includes, but are not limited to alkylamino, dialkylaminio, acylamino, and cycloalkylamino.

As used herein, the term “heterocycle” or “heterocyclyl” or “heterocycloalkyl” refers to unsubstituted and substituted mono- or polycyclic non-aromatic ring system containing one or more heteroatoms. Preferred heteroatoms include N, O, and S, including N-oxides, sulfur oxides, and dioxides. Preferably the ring is three to eight-membered and is either fully saturated or has one or more degrees of unsaturation. Multiple degrees of substitution are included within the present definition. Examples of “heterocyclic” groups include, but are not limited to tetrahydrofuranyl, pyranyl, 1,4-dioxanyl, 1,3-dioxanyl, piperidinyl, pyrrolidinyl, morpholinyl, azetidinyl, piperazinyl, pyrrolidinonyl, piperazinonyl, pyrazolidinyl, and their various tautomers, as well as unsubstituted and substituted versions thereof.

As used herein, the term “aryl”, unless otherwise defined, is meant aromatic, hydrocarbon, ring system. The ring system may be monocyclic or fused polycyclic (e.g., bicyclic, tricyclic, etc.), substituted or unsubstituted. In various embodiments, the monocyclic aryl ring is C5-C10, or C5-C7, or C5-C6, where these carbon numbers refer to the number of carbon atoms that form the ring system. A C6 ring system, i.e. a phenyl ring, is a suitable aryl group. In various embodiments, the polycyclic ring is a bicyclic aryl group, where suitable bicyclic aryl groups are C8-C12, or C9-C10. A naphthyl ring, which has 10 carbon atoms, is a suitable polycyclic aryl group. Suitable substituents for aryl are described in the definition of “optionally substituted”.

As used herein, the term “heteroaryl”, unless otherwise defined, is meant an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junctions, for example, bicyclic heteroaryl is a polycyclic heteroaryl. Bicyclic heteroaryl rings may contain from 8 to 12 member atoms. Monocyclic heteroaryl rings may contain from 5 to 8 member atoms (carbons and heteroatoms). Exemplary heteroaryl groups include: benzofuran, benzothiophene, furan, imidazole, indole, isothiazole, oxazole, pyrazine, pyrazole, pyridazine, pyridine, pyrimidine, pyrrole, quinoline, quinazoline, quinoxaline, thiazole, and thiophene. Suitable substituents for heteroaryl are described in the definition of “optionally substituted”.

As used herein, the term “cyano” refers to the group —CN.

As used herein, the term “acetyl” refers to the group —C(O)Rb, where Rb is alkyl, cycloalkyl, or heterocyclyl, as each is defined herein.

As used herein, the term “optionally” means that the subsequently described event(s) may or may not occur, and includes both event(s) that occur and event(s) that do not occur.

As used herein, unless otherwise defined, the phrase “optionally substituted” or variations thereof denote an optional substitution, including multiple degrees of substitution, with one or more substitutent group. The phrase should not be interpreted as duplicative of the substitutions herein described and depicted. Exemplary optional substituent groups include acyl, alkyl, alkylsulfonyl, alkoxy, alkoxycarbonyl, cyano, halogen, haloalkyl, hydroxyl, oxo, amide, sulfamide, urea, sulfonamide, thiourea and nitro.

The invention further provides a pharmaceutical composition (also referred to as pharmaceutical formulation) comprising a compound of Formula (I) or pharmaceutically acceptable salt, thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

In accordance with another aspect of the invention there is provided a process for the preparation of a pharmaceutical composition comprising mixing (or admixing) a compound of Formula (I) or salt thereof with at least one excipient.

### Pharmaceutical Compositions

Pharmaceutical compositions may be in unit dose form containing a predetermined amount of active ingredient per unit dose. Such a unit may contain a therapeutically effective dose of the compound of Formula (I) or salt thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound or salt thereof of the invention or the pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound or salt thereof of the invention in a suitably flavoured aqueous solution, while elixirs are prepared through the use of a non-toxic alcoholic vehicle. Suspensions can be formulated by dispersing the compound or salt of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

As used herein, the term “treatment” includes prophylaxis and refers to alleviating the specified condition, eliminating or reducing one or more symptoms of the condition, slowing or eliminating the progression of the condition, and preventing or delaying the reoccurrence of the condition in a previously afflicted or diagnosed patient or subject. Prophylaxis (or prevention or delay of disease onset) is typically accomplished by administering a drug in the same or similar manner as one would to a patient with the developed disease or condition.

The present invention provides a method of treatment in a mammal, especially a human, suffering from disease conditions targeted by the present compounds. Such treatment comprises the step of administering a therapeutically effective amount of a compound of Formula (I) or salt thereof to said mammal, particularly a human. Treatment can also comprise the step of administering a therapeutically effective amount of a pharmaceutical composition containing a compound of Formula (I) or salt thereof to said mammal, particularly a human.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. Additionally, the active ingredient may be presented as a pharmaceutical composition.

While it is possible that, for use in therapy, a therapeutically effective amount of a compound of Formula (I) or salt thereof may be administered as the raw chemical, it is typically presented as the active ingredient of a pharmaceutical composition or formulation.

The precise therapeutically effective amount of a compound or salt thereof of the invention will depend on a number of factors, including, but not limited to, the age and weight of the subject (patient) being treated, the precise disorder requiring treatment and its severity, the nature of the pharmaceutical formulation/composition, and route of administration, and will ultimately be at the discretion of the attending physician or veterinarian. Typically, a compound of Formula (I) or salt thereof will be given for the treatment in the range of about 0.1 to 100 mg/kg body weight of recipient (patient, mammal) per day and more usually in the range of 0.1 to 10 mg/kg body weight per day. Acceptable daily dosages may be from about 1 to about 1000 mg/day, and preferably from about 1 to about 100 mg/day. This amount may be given in a single dose per day or in a number (such as two, three, four, five, or more) of sub-doses per day such that the total daily dose is the same. An effective amount of a salt thereof may be determined as a proportion of the effective amount of the compound of Formula (I) per se. Similar dosages should be appropriate for treatment (including prophylaxis) of the other conditions referred herein for treatment. In general, determination of appropriate dosing can be readily arrived at by one skilled in medicine or the pharmacy art.

### Combinations

When a compound of Formula (I) is administered for the treatment of cancer, the term “co-administering” and derivatives thereof as used herein is meant either simultaneous administration or any manner of separate sequential administration of a FAS inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present FAS inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α, 4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects Occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10 S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4 S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

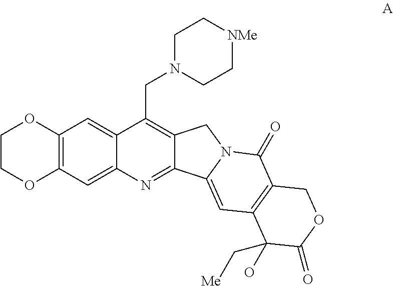

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor—I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta) IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bc1-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mc1-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the cancer treatment method of the claimed invention includes the co-administration a compound of Formula (I) and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

### EXPERIMENTALS

Abbreviations: BINAP, 2,2′-bis(diphenylphosphino)-1,1′-binaphthalene; Boc, t-butyloxycarbonyl; DCC, N,N′-dicyclohexylcarbodiimide; DCM, dichloromethane; DIEA, diisopropylethylamine; DMAP, 4-N,N-dimethylaminopyridine; DMF, N,N-dimethylformamide; DMSO, dimethylsulfoxide; EDC, N-(3-dimethylaminopropyl)-N′-ethylcarbodiimide hydrochloride; EtOAc, ethyl acetate; EtOH, ethanol; HOAc, acetic acid; HOAt, 1-hydroxy-7-azabenzotriazole; HOBt, 1-hydroxybenzotriazole; IPA, isopropyl alcohol; MeOH, methanol; TEA, triethylamine.

The racemic, Boc-protected aminomethylpyrrolidine A1 can be conveniently prepared from N1-benzyl-3-aminomethylpyrrolidine according to Scheme 1. The Boc-protected or acylated chiral aminomethylpyrrolidines A2 and A3 can be prepared from N-Boc-3-(R or S)-hydroxypyrrolidine according to Scheme 2.

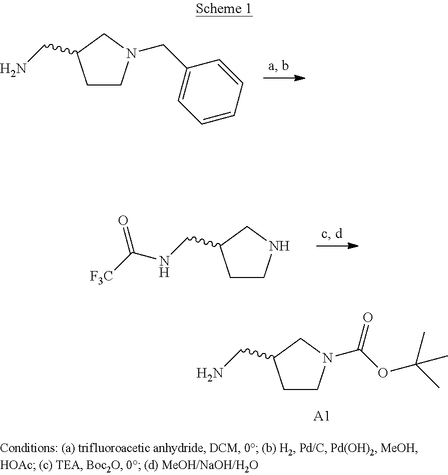

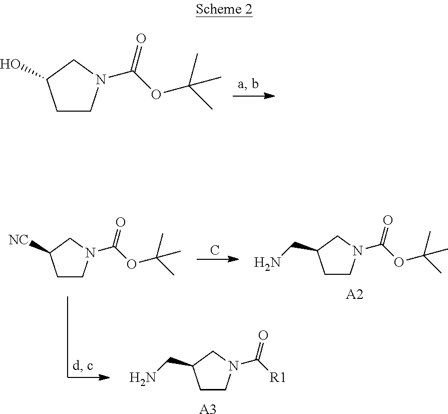

The 4-biarylcarbaldehydes can be prepared by Suzuki coupling of 4-formylarylboronic acids or esters with aryl bromides or Suzuki coupling of 4-formylarylbromides with arylboronic acids or esters, according to Scheme 3.

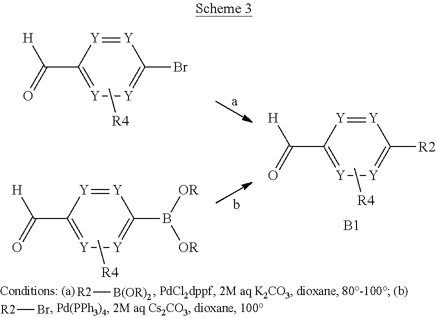

A typical synthesis begins with introduction of the aminomethylpyrrolidines A1, A2 or A3 via coupling with 2-halonitrobenzenes either by direct displacement or by copper- or palladium-catalyzed aryl amination as shown in Scheme 4. The Boc protecting group of A1 or A2 is removed with TFA or HCl and the pyrrolidine is acylated with an acyl chloride.

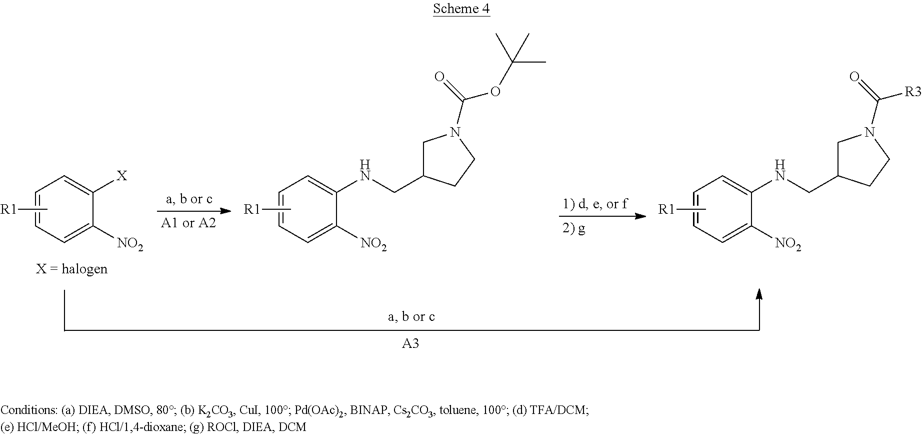

The aryl nitro group can be reduced by hydrogenation over Pd on carbon or by reduction with tin chloride as shown in Scheme 5. The resulting 1,2-phenylene diamine is condensed with biaryl aldehydes B1 to afford the final products.

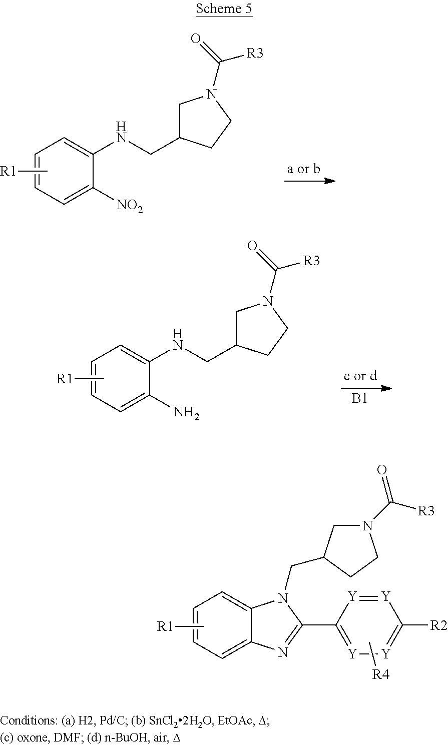

Alternatively, the benzimidazole ring can be formed directly from the nitroaniline intermediate by treatment with sodium hydrosulfite in the presence of the biaryl aldehyde B1 as shown in Scheme 6.

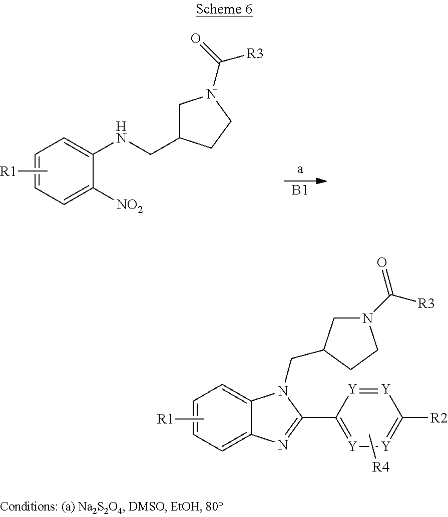

It is also possible to construct the biaryl moiety in a stepwise fashion by reacting the 1,2 phenylene diamine with a 4-bromo aryl aldehyde or 4-formylarylboronic acid to generate the benzimidazole and then performing a Suzuki coupling with the appropriate aryl boronic acid or aryl bromide as shown in Scheme 7. It is also possible to convert the intermediate bromophenylbenzimidazole to a boronate for subsequent Suzuki coupling with an aryl bromide.

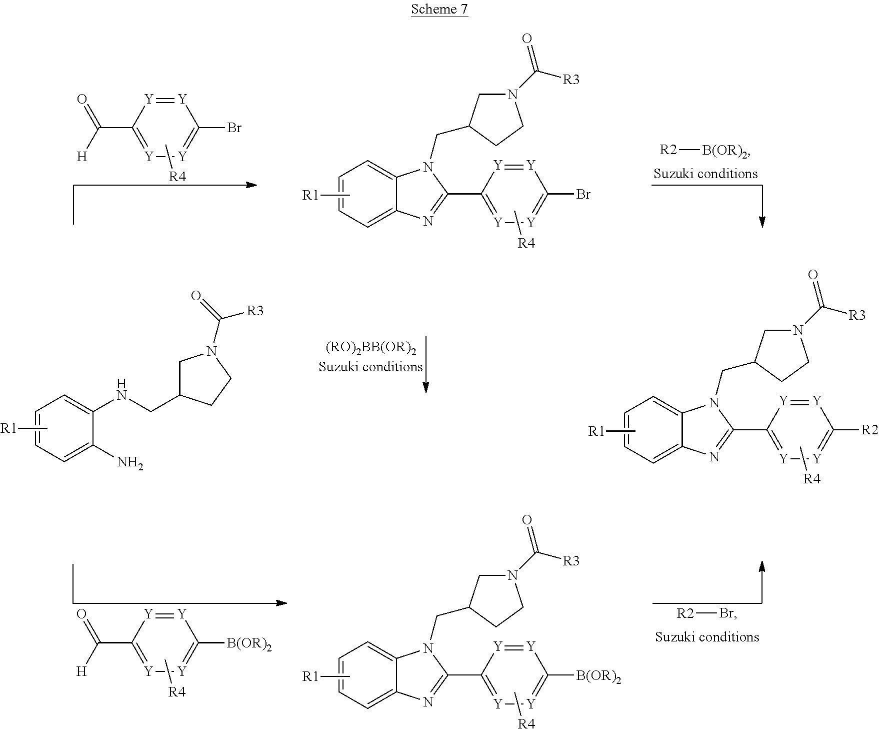

It is also possible to carry the Boc-protected pyrrolidine moiety through the benzimidazole formation, deprotect the pyrrolidine, and then cap the pyrrolidine moiety with acyl chlorides, chloroformates, carbamoyl chlorides or isocyanates to generate the corresponding acyl, carbamate or urea derivatives as shown in Scheme 8.

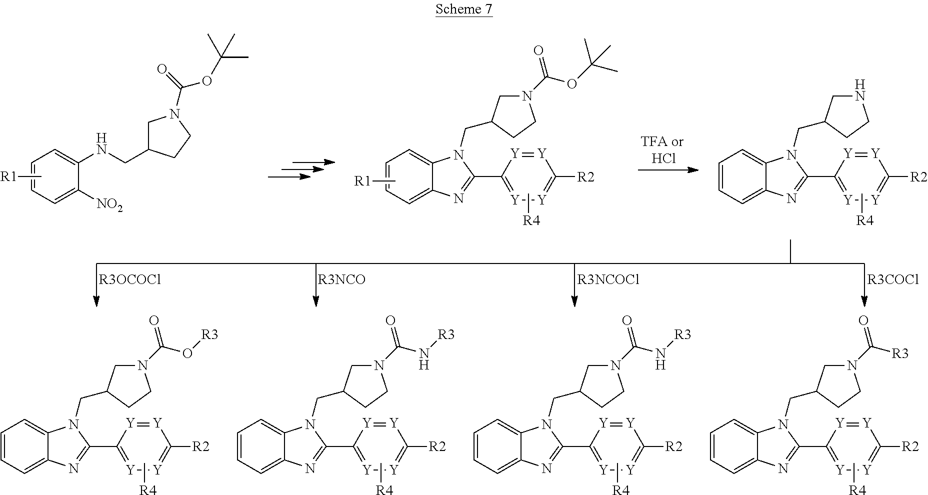

### Intermediate 1

### 1,1-Dimethylethyl (3RS)-3-(aminomethyl)-1-pyrrolidinecarboxylate

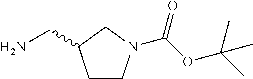

### (a) 2,2,2-Trifluoro-N-{[(3RS)-1-(phenylmethyl)-3-pyrrolidinyl]methyl}acetamide

N-benzyl-3(RS)-aminomethylpyrrolidine (160 g) was dissolved in 1.6 L DCM and cooled to 0° C. Trifluoroacetic anhydride (320 mL) was slowly added and the reaction mixture was allowed to warm to room temperature and stirred for 12 hr. The reaction mixture was washed with water (2×750 mL), brine (2×500 mL), dried over sodium sulfate and evaporated under reduced pressure to afford 190 g of the titled compound, which was used without further purification.

### (b) 2,2,2-Trifluoro-N-[(3RS)-3-pyrrolidinylmethyl]acetamide

2,2,2-trifluoro-N-[(3S)-3-pyrrolidinylmethyl]acetamide (190 g) was dissolved in 2.0 L MeOH. The flask was purged with nitrogen and 10% Pd/C (9.5 g), Pd(OH)2 (9.5 g) and glacial acetic acid (200 mL) were added. The reaction mixture was stirred under a hydrogen atmosphere for 2 days at RT. The reaction mixture was filtered through Celite and evaporated under reduced pressure to afford 200 g of the titled compound containing some residual acetic acid, which was used without further purification.

### (c) 1,1-Dimethylethyl (3RS)-3-{[(trifluoroacetyl)amino]methyl}-1-pyrrolidinecarboxylate

2,2,2-Trifluoro-N-[(3RS)-3-pyrrolidinylmethyl]acetamide (200 g) was dissolved in 2 L MeOH and cooled to 0° C. To this was slowly added TEA (284 mL) and the reaction mixture was allowed to stir at 0° C. for 30 min. Di-t-butyl dicarbonate (220 mL) was then slowly added and the reaction mixture was allowed to warm to RT and stirred for 1 day. The reaction mixture was concentrated and the residue dissolved in 1.5 L EtOAc. The EtOAc solution was washed with water (2×750 mL), brine (1×750 mL), dried over sodium sulfate and evaporated under reduced pressure. The crude product was purified by silica gel column chromatography using 10% EtOAc/90% petroleum ether to afford 150 g of the titled compound.

### (d) 1,1-Dimethylethyl (3RS)-3-(aminomethyl)-1-pyrrolidinecarboxylate

1,1-Dimethylethyl (3RS)-3-{[(trifluoroacetyl)amino]methyl}-1-pyrrolidinecarboxylate (150 g) was dissolved in 1 L MeOH. A 15% aqueous solution of NaOH (150 mL) was then added and the reaction mixture allowed to stir at RT for 3 hr. The reaction mixture was concentrated and the residue dissolved in 300 mL water. The aqueous solution was acidified by the addition of citric acid and extracted with diethyl ether (2×250 mL). The aqueous layer was then made basic by the addition of NaOH and extracted with diethyl ether (2×500 mL). These combined ether extracts were dried over sodium sulfate and evaporated under reduced pressure to afford 86 g of the titled compound.

### Intermediate 2

1,1-Dimethylethyl (3S)-3-(aminomethyl)-1-pyrrolidinecarboxylate

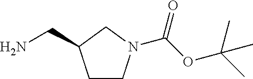

### a) 1,1-Dimethylethyl (3S)-3-[(methylsulfonyl)oxy]-1-pyrrolidinecarboxylate

A solution of 1,1-dimethylethyl (3S)-3-hydroxy-1-pyrrolidinecarboxylate (166 mmol) and N,N-diisopropylethylamine (265 mmol) in dichloromethane (200 mL) at 0° C. under nitrogen atmosphere was treated with methanesulfonyl chloride (199 mmol) in dichloromethane and allowed to warm to ambient over 1 h. Analysis by LCMS indicated the reaction was complete. The mixture was washed with 1M hydrochloric acid and brine, dried over sodium sulfate, filtered, and concentrated in vacuo. Purification of the residue by flash chromatography (0-5% methanol/dichloromethane) gave the title product in quantitative yield (166 mmol). 1H NMR (400 MHz, CDCl₃) δ ppm 1.49 (s, 9H) 2.08-2.21 (m, 1H) 2.29 (br. s., 1H) 3.07 (s, 3H) 3.36-3.64 (m, 3H) 3.65-3.75 (m, 1H) 5.28 (tt, J=4.23, 2.08 Hz, 1H).

### b) 1,1-Dimethylethyl (3R)-3-cyano-1-pyrrolidinecarboxylate

A mixture of 1,1-dimethylethyl (3S)-3-[(methylsulfonyl)oxy]-1-pyrrolidinecarboxylate (211 mmol) and sodium cyanide (633 mmol) in N,N-dimethylformamide (300 mL) was vigorously stirred with a mechanical stirrer while heating at 100° C. under a nitrogen atmosphere for 18 h. The mixture was allowed to cool to ambient temperature, filtered, and washed thoroughly with diethyl ether. The filtrate was diluted with dilute brine and extracted with diethyl ether (4×700 mL). The combined organic extracts were washed with dilute brine, filtered through a pad of sodium sulfate, and concentrated in vacuo. Purification of the residue by flash chromatography (0-50% ethyl acetate/hexanes) gave the title product (141 mmol, 67% yield). 1H NMR (400 MHz, CDCl₃) δ ppm 1.48 (s, 9H) 2.14-2.37 (m, 2H) 3.00-3.20 (m, 1H) 3.45 (dt, J=11.05, 6.98 Hz, 1H) 3.53-3.66 (m, 2 H) 3.65-3.76 (m, 1H).

### c) 1,1-Dimethylethyl (3S)-3-(aminomethyl)-1-pyrrolidinecarboxylate

A solution of 1,1-dimethylethyl (3R)-3-cyano-1-pyrrolidinecarboxylate (73.9 mmol) in ethanol (100 mL) was added to Raney Nickel (73.9 mmol; 2 scoops of Raney Nickel in water) in a Parr bottle under a stream of nitrogen. The mixture was well flushed with nitrogen then placed on a Parr shaker under hydrogen atmosphere at 60 psi for overnight. The mixture (under N2 stream) was filtered through Celite, washed with a little ethanol, and then the filter cake was immediately doused with water. The ethanol solution was concentrated in vacuo to afford the title product as a clear oil (71.9 mmol, 97% yield). The product was determined to be a 95:5 ratio of enantiomers (i.e., 90% ee). The use of chiral HPLC now (Daicel Chiralpak AD-H column (4.6×150 mm) with a mobile phase of heptane:ethanol:isopropylamine (85:10:0.1), a flow rate of 1.0 mL/min, and UV detection at 215 nm gave a retention time of 4.4 min for the 1,1-dimethylethyl (3R)-3-(aminomethyl)-1-pyrrolidinecarboxylate and 4.8 min for the 1,1-dimethylethyl (3S)-3-(aminomethyl)-1-pyrrolidinecarboxylate). 1H NMR (400 MHz, CDCl₃) δ ppm 1.47 (s, 9H) 1.54-1.73 (m, 1H) 1.91-2.13 (m, 1H) 2.28-2.52 (m, 1H) 2.78-2.93 (m, 1H) 2.95-3.16 (m, 1H) 3.24-3.67 (m, 5H).

### Intermediate 3

### {[(3S)-1-(Cyclopropylcarbonyl)-3-pyrrolidinyl]methyl}amine

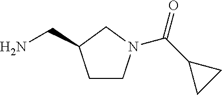

### a) (3R)-1-(Cyclopropylcarbonyl)-3-pyrrolidinecarbonitrile

A solution of 1,1-dimethylethyl (3R)-3-cyano-1-pyrrolidinecarboxylate (138 mmol) in ethanol (200 mL) was treated with 4N HCl in dioxane (480 mmol) and stirred for 2 h. The mixture was concentrated in vacuo to an oil and then azeotroped with ethanol and chloroform. The residue was dissolved in chloroform (300 mL) and treated with N,N-diisopropylethylamine (413 mmol) and cooled over an ice bath. The mixture was treated with cyclopropylcarbonyl chloride (165 mmol) in chloroform (100 mL) and then the ice bath was removed and the mixture stirred for 2 h. The mixture was washed with 1N hydrochloric acid and brine, dried over sodium sulfate, filtered, and concentrated in vacuo. Purification of the residue by flash chromatography (0-5% MeOH/DCM) gave the titled product (134 mmol, 97% yield). 1H NMR (400 MHz, CDCl₃) δ ppm 0.73-0.91 (m, 2H) 0.96-1.10 (m, 2H) 1.47-1.81 (m, 1H) 2.08-2.52 (m, 2H) 3.03-3.33 (m, 1H) 3.48-4.13 (m, 4H).

### b) {[(3S)-1-(Cyclopropylcarbonyl)-3-pyrrolidinyl]methyl}amine

A solution of (3R)-1-(cyclopropylcarbonyl)-3-pyrrolidinecarbonitrile (28.3 mmol) in ethanol (300 mL) and ammonia solution (15 mL, 28.3 mmol) was flushed with nitrogen and raney nickel catalyst (1 scoop) was added. The mixture was placed on a Parr shaker and flushed several times with nitrogen and then shaken under a hydrogen atmosphere at 60 psi for 3 h. The mixture was flushed with nitrogen and filtered through Celite under a nitrogen atmosphere (keeping the catalyst wet), and then the filter cake was washed with a little ethanol then immediately doused with water. The filtrate was evaporated to provide the crude product as a clear oil (25.6 mmol, 90% yield). Analysis by chiral HPLC indicated 75% of the title product, 2.4% of the other enantiomer, and ˜12% of the bis{[(3S)-1-(cyclopropylcarbonyl)-3-pyrrolidinyl]methyl}amine by-product. A sample of {[(3S)-1-(cyclopropylcarbonyl)-3-pyrrolidinyl]methyl}amine (1.06 g) was purified by chiral HPLC (Chiralpak AD 20μ column (101×250 mm) with a mobile phase of heptane:EtOH:isopropylamine (75:25:0.1), a flow rate of 500 mL/min, and UV detection at 220 nm gave a retention time of 8.5 min for the {[(3R)-1-(cyclopropylcarbonyl)-3-pyrrolidinyl]methyl}amine and 11 min for the {[(3S)-1-(cyclopropylcarbonyl)-3-pyrrolidinyl]methyl}amine) to afford 720 mg (68% recovery) of chirally (>99% ee) and chemically pure title compound. 1H NMR (400 MHz, CDCl₃) δ ppm 0.69-0.83 (m, 2H) 0.91-1.09 (m, 2H) 1.28 (br. s., 2H) 1.52-1.82 (m, 2H) 1.97-2.20 (m, 1H) 2.20-2.45 (m, 1H) 2.67-2.98 (m, 2H) 3.06-3.38 (m, 1H) 3.38-3.92 (m, 3H).

### Intermediate 4

### 4-(1,3-Benzothiazol-6-yl)benzaldehyde

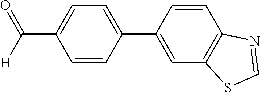

4-Formylphenylboronic acid (150 mg) and 6-bromo-1,3-benzothiazole (210 mg) were dissolved in 5 mL 1,4-dioxane and the solution was degassed and flushed with nitrogen several times. To this was added tetrakis(triphenylphosphine)palladium (58 mg) and K2CO3 (0.56 g, 4 eq) in 2 mL water. The reaction mixture was degassed and flushed again with nitrogen and stirred at 100° C. overnight. The reaction mixture was allowed to cool then evaporated to dryness, dissolved in a minimum amount of EtOAc, filtered and purified by preparative tlc to yield the titled compound.

### Intermediate 5

### 4-(1H-Indol-6-yl)benzaldehyde

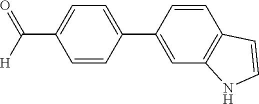

6-Bromoindole (6.0 g), 4-formylphenylboronic acid (6.72 g), Na2CO3 (8.1 g, 3 eq), and tetrakis(triphenylphosphine)palladium (0.35 g) were suspended in 180 mL 1,4-dioxane and 30 mL water and heated at 80° C. overnight. Solvent was removed by evaporation and the crude product was purified by preparative reverse phase HPLC followed by preparative TLC to afford the titled compound.

### Intermediate 6

### 5-(4-Formylphenyl)-1H-indazole

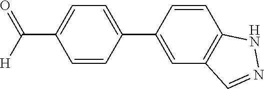

### (a) 1,1-Dimethylethyl 5-bromo-1H-indazole-1-carboxylate

To a stirred, ice-cold suspension of 5-bromoindazole (197 mg), 4-dimethylaminopyridine (24 mg,) and triethylamine (0.2 mL,) in 2.5 mL acetonitrile was added di-t-butyl dicarbonate (218 mg) in 2.5 mL acetonitrile dropwise over 15 min. so that the temperature remained below 5° C. The reaction mixture was allowed to warm to RT and stirred for 18 hr. The resulting slurry was purified by silica gel column chromatography using petroleum ether/EtOAc 15:1 as eluant to afford 163 mg of the titled compound.

### (b) 5-(4-Formylphenyl)-1H-indazole

1,1-Dimethylethyl 5-bromo-1H-indazole-1-carboxylate (8.48 g), 4-formylphenylboronic acid (5.76 g), tetrakis(triphenylphosphine)palladium (1.65 g) and 228 mL 2 M aqueous Cs2CO3 were suspended in 228 mL 1,4-dioxane under N2 and heated to 80° C. overnight. After removal of solvent, the crude product was purified by preparative TLC to afford 2.2 g of the titled compound.

### Intermediate 7

### 6-(4-Formylphenyl)-1H-indazole

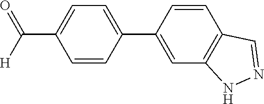

### (a) 1,1-Dimethylethyl 6-bromo-1H-indazole-1-carboxylate

The titled compound was prepared according to the procedure in Intermediate 6(a), substituting 6-bromoindazole.

### (b) 6-(4-Formylphenyl)-1H-indazole

The titled compound was prepared according to the procedure in Intermediate 6(b), substituting 1,1-dimethylethyl 6-bromo-1H-indazole-1-carboxylate.

### Intermediate 8

### (a) 4-(1-Benzofuran-5-yl)benzaldehyde

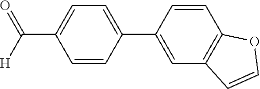

5-Bromo-1-benzofuran (5 g), 4-formylphenylboronic acid (3.8 g, 1 eq), K2CO3 (7 g, 2 eq) were dissolved in 50 mL 1,4-dioxane and 20 mL water. Pd(dppf)C12 (2.2 g, 0.1 eq) was added under nitrogen. The reaction mixture was degassed and flushed with nitrogen 3 times and then stirred at 80° C. overnight. The reaction mixture was allowed to cool and the solvent was removed by evaporation. The residue was dissolved in EtOAc and purified by silica gel column chromatography to afford 2.0 g of the titled compound.

### Example 1

### 1-{[(3S)-1-(Cyclopropylcarbonyl)-3-pyrrolidinyl]methyl}-2-[4-(1H-indol-5-yl)phenyl]-1H-benzimidazole

### (a) 1,1-Dimethylethyl (3S)-3-{[(2-nitrophenyl)amino]methyl}-1-pyrrolidinecarboxylate

2-Fluoronitrobenzene (1.42 g) and 1,1-dimethylethyl (3S)-3-(aminomethyl)-1-pyrrolidinecarboxylate (2.32 g) were dissolved in 8 mL DMF and heated at 80° C. for 15 hrs. The reaction mixture was cooled, diluted with 60 mL water and extracted with EtOAc. The extracts were filtered through a plug of silica gel, evaporated to dryness and the residue dried under high vacuum to yield the titled compound, which was used without further purification.

### (b) 1,1-Dimethylethyl (3S)-3-{[(2-aminophenyl)amino]methyl}-1-pyrrolidinecarboxylate

1,1-Dimethylethyl (3S)-3-{[(2-nitrophenyl)amino]methyl}-1-pyrrolidinecarboxylate (2.24 g) and 10% Pd/C (100 mg) were suspended in 45 mL EtOH/1.6 mL glacial HOAc and hydrogenated on a Parr shaker at 50 psi H2 for 12 hrs. The reaction mixture was filtered through a plug of alumina and the filtrate evaporated to dryness to yield the titled compound (LCMS m/z 192, M+H-Boc), which was used without further purification.

### (c) 1,1-Dimethylethyl (3S)-3-{[2-(4-bromophenyl)-1H-benzimidazol-1-yl]methyl}-1-pyrrolidinecarboxylate

1,1-Dimethylethyl (3S)-3-{[(2-aminophenyl)amino]methyl}-1-pyrrolidinecarboxylate (2.0 g) and 4-bromobenzaldehyde (1.34 g) were dissolved in 50 mL 1-butanol and heated at 80° C. for 15 hrs. The reaction mixture was allowed to cool and evaporated to dryness. The crude product was dissolved in a small amount of DCM and purified by flash chromatography on silica gel using a gradient of 0-50% EtOAc in DCM. The appropriate fractions were pooled and evaporated to dryness. This partially purified material was again dissolved in a small amount of DCM and purified again by flash chromatography on silica gel using a gradient of 0-30% EtOAc in DCM. The appropriate fractions were combined and evaporated to dryness to yield the titled compound (LCMS m/z 456, M+H), which was used without further purification.

### (d) 2-(4-Bromophenyl)-1-{[(3S)-1-(cyclopropylcarbonyl)-3-pyrrolidinyl]methyl}-1H-benzimidazole

1,1-Dimethylethyl (3S)-3-{[2-(4-bromophenyl)-1H-benzimidazol-1-yl]methyl}-1-pyrrolidinecarboxylate (2.3 g) was dissolved in 6 mL THF. To this was added 3 mL conc. HCl and the reaction was stirred at RT for 2 hrs. The reaction mixture was diluted with saturated brine, the pH was adjusted to ˜8 with 1 N KOH and extracted with EtOAc (3×100 mL). The combined extracts were dried over sodium sulfate and evaporated to dryness to yield 1.48 g of 2-(4-bromophenyl)-1-[(3R)-3-pyrrolidinylmethyl]-1H-benzimidazole hydrochloride salt. A 510-mg aliquot of this amine hydrochloride salt was dissolved in 6 mL THF. To this was added cyclopropylcarbonyl chloride (156 uL) and triethylamine (240 uL) and the reaction was stirred at RT for 5 min. The reaction mixture was diluted with water, the pH adjusted to ˜2 with 1 N HCl and extracted with EtOAc (3×20 mL). The pH of the aqueous layer was brought to 8 with 1 N NaHCO3 and extracted again with EtOAc (2×20 mL). The combined organic extracts were dried over sodium sulfate and evaporated to dryness to yield the titled compound (LCMS m/z 424, M+H), which was used without further purification.

### (e) 1-{[(3S)-1-(Cyclopropylcarbonyl)-3-pyrrolidinyl]methyl}-2-[4-(1H-indol-5-yl)phenyl]-1H-benzimidazole

2-(4-Bromophenyl)-1-{[(3S)-1-(cyclopropylcarbonyl)-3-pyrrolidinyl]methyl}-1H-benzimidazole (48 mg), 5-indolylboronic acid (26 mg), tetrakis(triphenylphosphonato)palladium (1.3 mg) and sodium carbonate (16 mg) were dissolved in 2 mL DMF and 0.5 mL water under a nitrogen atmosphere and were heated to 80° C. for 12 hrs. The reaction was not complete so an additional 1.3 mg tetrakis(triphenylphosphonato)palladium was added and the reaction stirred an additional 12 hrs at 80° C. The reaction mixture was allowed to cool and then directly purified by preparative reverse phase HPLC. The appropriate fractions were combined and solvent was removed on a Genevac centrifugal evaporator to yield the titled compound (LCMS m/z 461, M+H).

### Example 2

### 1-{[(3S)-1-(Cyclopropylcarbonyl)-3-pyrrolidinyl]methyl}-2-[4-(1H-indol-6-yl)phenyl]-1H-benzimidazole

2-(4-Bromophenyl)-1-{[(3S)-1-(cyclopropylcarbonyl)-3-pyrrolidinyl]methyl}-1H-benzimidazole (50 mg), indole-6-boronic acid (27 mg), tetrakis(triphenylphosphine)palladium (1.3 mg) and sodium carbonate (18 mg) were dissolved in 2 mL DMF and 0.5 mL water under a nitrogen atmosphere and were heated to 80° C. for 12 hrs. The reaction was not complete so an additional 1.3 mg tetrakis(triphenylphosphine)palladium was added and the reaction stirred an additional 12 hrs at 80° C. The reaction mixture was purified by preparative reverse phase HPLC. The appropriate fractions were combined and solvent was removed on a Genevac centrifugal evaporator to yield the titled compound (LCMS m/z 461, M+H).

### Example 3

### 2-[4-(1-Benzofuran-5-yl)phenyl]-1-{[(3S)-1-(cyclopropylcarbonyl)-3-pyrrolidinyl]methyl}-1H-benzimidazole

2-(4-Bromophenyl)-1-{[(3S)-1-(cyclopropylcarbonyl)-3-pyrrolidinyl]methyl}-1H-benzimidazole (181 mg), 5-(4,4,5,5-tetramethyl-1,3-dioxaborolan-2-yl)-1-benzofuran (125 mg), tetrakis(triphenylphosphine)palladium (98 mg) and sodium carbonate (54 mg) were suspended in 3 mL DMF and 0.7 mL water under nitrogen and heated to 80° C. for 1.5 hr. The reaction mixture was allowed to cool to RT and then was diluted with 0.5 mL MeOH. The reaction mixture was filtered and purified by preparative reverse phase HPLC. The appropriate fractions were combined and evaporated to dryness on a Genevac centrifugal evaporator to yield the titled compound (LCMS m/z 462, M+H).

### Example 4

### 6-[4-(1-{[(3S)-1-(Cyclopropylcarbonyl)-3-pyrrolidinyl]methyl}-1H-benzimidazol-2-yl)phenyl]-1,3-benzothiazole

### (a) N-{[(3S)-1-(Cyclopropylcarbonyl)-3-pyrrolidinyl]methyl}-2-nitroaniline

1,1-Dimethylethyl (3S)-3-{[(2-nitrophenyl)amino]methyl}-1-pyrrolidinecarboxylate (2.6 g) was dissolved in 20 mL 5 N HCl/MeOH and stirred at RT for 3 hr. Evaporation of the solvent yielded the intermediate 2-nitro-N-[(3R)-3-pyrrolidinylmethyl]aniline hydrochloride salt. This material was dissolved in 25 mL DCM, and DIEA (3.38 g) was added. The reaction mixture was stirred at RT for 30 min. Cyclopropylcarbonyl chloride (1.10 g) was then added dropwise and the reaction mixture stirred at RT overnight. Saturated aqueous NaHCO3 was then added dropwise and the reaction mixture extracted with EtOAc. The EtOAc extracts were washed with water and saturated brine, dried over sodium sulfate, and evaporated to dryness to yield the titled compound, which was used without further purification.

### (b) N-{[(3S)-1-(Cyclopropylcarbonyl)-3-pyrrolidinyl]methyl}-1,2-benzenediamine

N-{[(3S)-1-(Cyclopropylcarbonyl)-3-pyrrolidinyl]methyl}-2-nitroaniline (2.34 g) was dissolved in 30 mL MeOH and 1.6 g 10% Pd/C was added under argon. The reaction mixture was stirred under a hydrogen atmosphere overnight at RT. The reaction mixture was filtered and the filtrate evaporated to dryness to yield the titled compound, which was used without further purification.

### (c) 6-[4-(1-{[(3S)-1-(Cyclopropylcarbonyl)-3-pyrrolidinyl]methyl}-1H-benzimidazol-2-yl)phenyl]-1,3-benzothiazole

To a solution of N-{[(3S)-1-(cyclopropylcarbonyl)-3-pyrrolidinyl]methyl}-1,2-benzenediamine (150 mg) in 4 mL DMF was added 4-(1,3-benzothiazol-6-yl)benzaldehyde (152 mg) and oxone (231 mg) and the reaction was stirred at RT overnight. The reaction mixture was filtered and the filtrate purified by preparative reverse phase HPLC to yield 100 mg of the titled compound (LCMS m/z 479.3, M+H).

### Example 5

### 1-{[(3S)-1-(Cyclopropylcarbonyl)-3-pyrrolidinyl]methyl}-2-(4′-fluoro-4-biphenylyl)-4-(methyloxy)-1H-benzimidazole

### (a) 1,1-Dimethylethyl (3S)-3-({[3-(methyloxy)-2-nitrophenyl]amino}methyl)-1-pyrrolidinecarboxylate

1-Fluoro-3-(methyloxy)-2-nitrobenzene (4.5 g) was dissolved in 20 mL dry DMSO. To this was added 1,1-dimethylethyl (3S)-3-(aminomethyl)-1-pyrrolidinecarboxylate (6.8 g) and DIEA (4.7 g) in 30 mL dry DMSO and the reaction mixture was stirred at 70° C. overnight. The reaction mixture was allowed to cool and then was diluted with water and extracted several times with DCM. The combined organic extracts were washed with saturated brine, dried over sodium sulfate, and evaporated to dryness to give the crude product. The crude product was purified by column chromatography on silica gel eluted with petroleum ether/EtOAc (40:1).

### (b) N-{[(3S)-1-(Cyclopropylcarbonyl)-3-pyrrolidinyl]methyl}-3-(methyloxy)-2-nitroaniline

1,1-Dimethylethyl (3S)-3-({[3-(methyloxy)-2-nitrophenyl]amino}methyl)-1-pyrrolidinecarboxylate (8.5 g) was dissolved in 5 mL MeOH. To this was added 20 mL of 5 M HCl/MeOH dropwise and the reaction mixture was stirred at RT for 2 hr. The solvent was removed by evaporation to yield the intermediate 3-(methyloxy)-2-nitro-N-[(3R)-3-pyrrolidinylmethyl]aniline hydrochloride salt. This material was dissolved in 40 mL dry DCM. To this was added DIEA (7.6 g) and the reaction mixture stirred at RT for 30 min. Cyclopropylcarbonyl chloride (2.7 g) was then added dropwise and the reaction mixture stirred at RT overnight. The reaction mixture was partitioned between DCM and saturated aqueous NaHCO3. The organic layer was washed with saturated brine, dried over sodium sulfate, and evaporated to dryness. The crude product was purified by column chromatography on silica gel, eluting with petroleum ether/EtOAc (1:1) to yield the titled compound (LCMS m/z 320.1, M+H).

### (c) [2-Amino-3-(methyloxy)phenyl]{[(3S)-1-(cyclopropylcarbonyl)-3-pyrrolidinyl]methyl}amine

N-{[(3S)-1-(Cyclopropylcarbonyl)-3-pyrrolidinyl]methyl}-3-(methyloxy)-2-nitroaniline (5 g) was dissolved in 30 mL MeOH. To this was added 10% Pd/C (2.0 g) and the reaction was stirred under a hydrogen atmosphere at RT for 3 hr. The reaction mixture was filtered and the solvent evaporated to yield the titled compound (LCMS m/z 300.0, M+H), which was used without further purification.

### (d) 1-{[(3S)-1-(Cyclopropylcarbonyl)-3-pyrrolidinyl]methyl}-2-(4′-fluoro-4-biphenylyl)-4-(methyloxy)-1H-benzimidazole

[2-Amino-3-(methyloxy)phenyl]{[(3S)-1-(cyclopropylcarbonyl)-3-pyrrolidinyl]methyl}amine (120 mg) was dissolved in 3 mL DMF. To this was added 4-(4′-fluorophenyl)benzaldehyde (82 mg) followed by oxone (165 mg) and the reaction mixture was stirred overnight at RT. A mixture of 5 mL 1 M K2CO3 and 30 mL water was added to the reaction mixture dropwise with vigorous stirring. The reaction mixture was extracted with EtOAc (60 mL) and the organic extract was washed with water and saturated brine, dried over sodium sulfate, and evaporated to dryness. The crude product was purified by preparative TLC to afford 130 mg of the titled compound (LCMS m/z 470.3, M+H).

### Example 6

### 1-{[(3S)-1-(Cyclopropylcarbonyl)-3-pyrrolidinyl]methyl}-4-(methyloxy)-2-[4′-(methyloxy)-4-biphenylyl]-1H-benzimidazole

The titled compound was prepared according to the procedure in Example 5(d), substituting 4-(4′-methoxyphenyl)benzaldehyde, to afford 87 mg of the desired product (LCMS m/z 482.3).

### Example 7

### 1-{[(3S)-1-(Cyclopropylcarbonyl)-3-pyrrolidinyl]methyl}-2-[4-(1H-indol-5-yl)phenyl]-4-(methyloxy)-1H-benzimidazole

The titled compound was prepared according to the procedure in Example 5(d), substituting 4-(1H-indol-5-yl)benzaldehyde, to afford 65 mg of the desired product (LCMS m/z 491.1, M+H).

### Example 8

### 1-{[(3S)-1-(Cyclopropylcarbonyl)-3-pyrrolidinyl]methyl}-2-[4-(1H-indol-6-yl)phenyl]-4-(methyloxy)-1H-benzimidazole

The titled compound was prepared according to the procedure in Example 5(d), substituting 4-(1H-indol-6-yl)benzaldehyde, to afford 43 mg of the desired product (LCMS m/z 491.2, M+H).

### Example 9

### 5-{4-[1-{[(3S)-1-(Cyclopropylcarbonyl)-3-pyrrolidinyl]methyl}-4-(methyloxy)-1H-benzimidazol-2-yl]phenyl}-1H-indazole

The titled compound was prepared according to the procedure in Example 5(d), substituting 5-(4-formylphenyl)-1H-indazole, to afford 73 mg of the desired product (LCMS m/z 492.2, M+H).

### Example 10

### 6-{4-[1-{[(3S)-1-(Cyclopropylcarbonyl)-3-pyrrolidinyl]methyl}-4-(methyloxy)-1H-benzimidazol-2-yl]phenyl}-1H-indazole

The titled compound was prepared according to the procedure in Example 5(d), substituting 6-(4-formylphenyl)-1H-indazole, to afford 17 mg of the desired product (LCMS m/z 492.3, M+H).

### Example 11

### 2-(4-Biphenylyl)-1-{[(3S)-1-(cyclopropylcarbonyl)-3-pyrrolidinyl]methyl}-4-(methyloxy)-1H-benzimidazole

The titled compound was prepared according to the procedure in Example 5(d), substituting 4-biphenylcarbaldehyde, to afford 115 mg of the desired product (LCMS m/z 452.4, M+H).

### Example 12

### 2-[4-(1-Benzofuran-5-yl)phenyl]-1-{[(3S)-1-(cyclopropylcarbonyl)-3-pyrrolidinyl]methyl}-4-(methyloxy)-1H-benzimidazole

The titled compound was prepared according to the procedure in Example 5(d), substituting 4-(1-benzofuran-5-yl)benzaldehyde, to afford 55 mg of the desired product (LCMS m/z 492.3, M+H).

### Example 13

### 1-{[(3S)-1-(Cyclopropylcarbonyl)-3-pyrrolidinyl]methyl}-2-(4′-fluoro-4-biphenylyl)-4-methyl-1H-benzimidazole

### (a) 1,1-Dimethylethyl (3S)-3-{[(3-methyl-2-nitrophenyl)amino]methyl}-1-pyrrolidinecarboxylate

3-Methyl-2-nitrofluorobenzene, 1,1-dimethylethyl (3S)-3-(aminomethyl)-1-pyrrolidinecarboxylate (1 eq) and DIEA (1.4 eq) were dissolved in DMSO and stirred at 70° C. overnight. The reaction mixture was allowed to cool, diluted with EtOAc, washed with water and saturated brine, dried over sodium sulfate and evaporated to yield the crude product. Purification by silica gel column chromatography afforded the titled compound.

### (b) N-{[(3S)-1-(Cyclopropylcarbonyl)-3-pyrrolidinyl]methyl}-3-methyl-2-nitroaniline

1,1-Dimethylethyl (3S)-3-{[(3-methyl-2-nitrophenyl)amino]methyl}-1-pyrrolidinecarboxylate was dissolved in 5 N HCl/MeOH and stirred for 2 hr at RT. Removal of the solvent gave the intermediate amine hydrochloride salt, which was dissolved in DCM. Cyclopropylcarbonyl chloride (1.2 eq) and DIEA (2.5 eq) were added and the reaction was stirred at RT until complete. The reaction mixture was diluted with EtOAc, washed with water and saturated brine, dried over sodium sulfate and evaporated to dryness. The crude product was purified by silica gel column chromatography to afford the titled compound.

### (c) N1-{[(3S)-1-(Cyclopropylcarbonyl)-3-pyrrolidinyl]methyl}-3-methyl-1,2-benzenediamine

N-{[(3S)-1-(Cyclopropylcarbonyl)-3-pyrrolidinyl]methyl}-3-methyl-2-nitroaniline was dissolved in EtOH. To this was added 10% Pd/C and the reaction was stirred under a hydrogen atmosphere (35 psi) at RT overnight. The reaction mixture was filtered and the filtrate evaporated to dryness to afford the titled compound, which was used without further purification.

### (d) 1-{[(3S)-1-(Cyclopropylcarbonyl)-3-pyrrolidinyl]methyl}-2-(4′-fluoro-4-biphenylyl)-4-methyl-1H-benzimidazole

N1-{[(3S)-1-(Cyclopropylcarbonyl)-3-pyrrolidinyl]methyl}-3-methyl-1,2-benzenediamine (90 mg) was reacted with 4-(4′-fluorophenyl)benzaldehyde according to the procedure in Example 5(d). Purification by preparative reverse phase HPLC afforded 63 mg of the titled compound (LCMS m/z 454.3, M+H).

### Example 14

### 1-{[(3S)-1-(Cyclopropylcarbonyl)-3-pyrrolidinyl]methyl}-4-methyl-2-[4′-(methyloxy)-4-biphenylyl]-1H-benzimidazole

The titled compound was prepared according to the procedure in Example 13(d), substituting 4-(4′-methoxyphenyl)benzaldehyde, to afford 46 mg of the desired product (LCMS m/z 466.3, M+H).

### Example 15

### 2-[4-(1-Benzofuran-5-yl)phenyl]-1-{[(3S)-1-(cyclopropylcarbonyl)-3-pyrrolidinyl]methyl}-4-methyl-1H-benzimidazole

The titled compound was prepared according to the procedure in Example 13(d), substituting 4-(1-benzofuran-5-yl)benzaldehyde, to afford 98 mg of the desired product (LCMS m/z 476.3, M+H).

### Example 16

### 1-{[(3S)-1-(Cyclopropylcarbonyl)-3-pyrrolidinyl]methyl}-2-[4-(1H-indol-5-yl)phenyl]-4-methyl-1H-benzimidazole

The titled compound was prepared according to the procedure in Example 13(d), substituting 4-(1H-indol-5-yl)benzaldehyde, to afford 25 mg of the desired product (LCMS m/z 475.4, m+H).

### Example 17

### 1-{[(3S)-1-(Cyclopropylcarbonyl)-3-pyrrolidinyl]methyl}-2-[4-(1H-indol-6-yl)phenyl]-4-methyl-1H-benzimidazole

The titled compound was prepared according to the procedure in Example 13(d), substituting 4-(1H-indol-6-yl)benzaldehyde, to afford 48 mg of the desired product (LCMS m/z 475.3, M+H).

### Example 18

### 5-[4-(1-{[(3S)-1-(Cyclopropylcarbonyl)-3-pyrrolidinyl]methyl}-4-methyl-1H -benzimidazol-2-yl)phenyl]-1H-indazole

The titled compound was prepared according to the procedure in Example 13(d), substituting 5-(4-formylphenyl)-1H-indazole, to afford 53 mg of the desired product (LCMS m/z 476.4, M+H).

### Example 19

### 6-[4-(1-{[(3S)-1-(Cyclopropylcarbonyl)-3-pyrrolidinyl]methyl}-4-methyl-1H -benzimidazol-2-yl)phenyl]-1H-indazole

The titled compound was prepared according to the procedure in Example 13(d), substituting 6-(4-formylphenyl)-1H-indazole, to afford 48 mg of the desired product (LCMS m/z 476.3, M+H).

### Example 20

### 2-(4-Biphenylyl)-1-{[(3S)-(cyclopropylcarbonyl)-3-pyrrolidinyl]methyl}-4-methyl-1H-benzimidazole

The titled compound was prepared according to the procedure in Example 13(d), substituting 4-biphenylcarbaldehyde, to afford 87 mg of the desired product (LCMS m/z 436.3, M+H).

### Example 21

### 1-{[(3S)-1-(Cyclopropylcarbonyl)-3-pyrrolidinyl]methyl}-2-(4′-fluoro-4-biphenylyl)-4-(trifluoromethyl)-1H-benzimidazole

### (a) 1,1-Dimethylethyl (3S)-3-({[2-nitro-3-(trifluoromethyl)phenyl]amino}methyl)-1-pyrrolidinecarboxylate

1-Chloro-2-nitro-3-(trifluoromethyl)benzene (3 g) was dissolved in 15 mL DMSO. To this was added 1,1-dimethylethyl (3S)-3-(aminomethyl)-1-pyrrolidinecarboxylate (3.5 g) and DIEA (2.4 g) in 15 mL DMSO in one portion, and the reaction mixture was stirred at 70° C. overnight. The reaction mixture was allowed to cool, diluted with water and extracted several times with EtOAc. The combined EtOAc extracts were washed with saturated brine. After removal of the solvent by evaporation, the crude product was purified by silica gel column chromatography to afford the titled compound.

### (b) N-{[(3S)-1-(Cyclopropylcarbonyl)-3-pyrrolidinyl]methyl}-2-nitro-3-(trifluoromethyl)aniline

The titled compound was prepared from 1,1-dimethylethyl (3S)-3-({[2-nitro-3-(trifluoromethyl)phenyl]amino}methyl)-1-pyrrolidinecarboxylate according to the procedure in Example 13(b).

### (c) N¹-{[(3S)-1-(Cyclopropylcarbonyl)-3-pyrrolidinyl]methyl}-3-(trifluoromethyl)-1,2-benzenediamine

The titled compound was prepared from N-{[(3S)-1-(cyclopropylcarbonyl)-3-pyrrolidinyl]methyl}-2-nitro-3-(trifluoromethyl)aniline according to the procedure in Example 13(c).

### (d) 1-{[(3S)-1-(Cyclopropylcarbonyl)-3-pyrrolidinyl]methyl}-2-(4′-fluoro-4-biphenylyl)-4-(trifluoromethyl)-1H-benzimidazole

The titled compound was prepared from N1-{[(3S)-1-(cyclopropylcarbonyl)-3-pyrrolidinyl]methyl}-3-(trifluoromethyl)-1,2-benzenediamine and 4-(4′-fluorophenyl)benzaldehyde according to the procedure in Example 13(d), to afford 64 mg of the desired product (LCMS m/z 508.3, M+H).

### Example 22

### 1-{[(3S)-1-(Cyclopropylcarbonyl)-3-pyrrolidinyl]methyl}-2-[4′-(methyloxy)-4-biphenylyl]-4-(trifluoromethyl)-1H-benzimidazole

The titled compound was prepared according to the procedure in Example 21(d), substituting 4-(4′-methoxyphenyl)benzaldehyde, to afford 86 mg of the desired product (LCMS m/z 520.3, M+H).

### Example 23

### 1-{[(3S)-1-(Cyclopropylcarbonyl)-3-pyrrolidinyl]methyl}-2-[4-(1H-indol-5-yl)phenyl]-4-(trifluoromethyl)-1H-benzimidazole

The titled compound was prepared according to the procedure in Example 21(d), substituting 4-(1H-indol-5-yl)benzaldehyde, to afford 65 mg of the desired product (LCMS m/z 529.3, M+H).

### Example 24

### 1-{[(3S)-1-(Cyclopropylcarbonyl)-3-pyrrolidinyl]methyl}-2-[4-(1H-indol-6-yl)phenyl]-4-(trifluoromethyl)-1H-benzimidazole

The titled compound was prepared according to the procedure in Example 21(d), substituting 4-(1H-Indol-6-yl)benzaldehyde, to afford 50 mg of the desired product (LCMS m/z 529.3, M+H).

### Example 25

### 5-{4-[1-{[(3S)-1-(Cyclopropylcarbonyl)-3-pyrrolidinyl]methyl}-4-(trifluoromethyl)-1H-benzimidazol-2-yl]phenyl}-1H-indazole

The titled compound was prepared according to the procedure in Example 21(d), substituting 5-(4-formylphenyl)-1H-indazole, to afford 48 mg of the desired product (LCMS m/z 530.1, M+H).

### Example 26

### 6-{4-[1-{[(3S)-1-(Cyclopropylcarbonyl)-3-pyrrolidinyl]methyl}-4-(trifluoromethyl)-1H-benzimidazol-2-yl]phenyl}-1H-indazole

The titled compound was prepared according to the procedure in Example 21(d), substituting 6-(4-formylphenyl)-1H-indazole, to afford 43 mg of the desired product (LCMS m/z 530.2, M+H).

### Example 27

### 2-[4-(1-Benzofuran-5-yl)phenyl]-1-{[(3S)-1-(cyclopropylcarbonyl)-3-pyrrolidinyl]methyl}-4-(trifluoromethyl)-1H-benzimidazole

The titled compound was prepared according to the procedure in Example 21(d), substituting 4-(1-benzofuran-5-yl)benzaldehyde, to afford 75 mg of the desired product (LCMS m/z 530.3, M+H).

### Example 28

### 2-(4-Biphenylyl)-1-{[(3S)-1-(cyclopropylcarbonyl)-3-pyrrolidinyl]methyl}-4-(trifluoromethyl)-1H-benzimidazole

The titled compound was prepared according to the procedure in Example 21(d), substituting 4-biphenylcarbaldehyde, to afford 64 mg of the desired product (LCMS m/z 490.1, M+H).

### Example 29

### 4-Bromo-1-{[(3S)-1-(cyclopropylcarbonyl)-3-pyrrolidinyl]methyl}-2-(4′-fluoro-4-biphenylyl)-1H-benzimidazole

### (a) 1,1-Dimethylethyl (3S)-3-{[(3-bromo-2-nitrophenyl)amino]methyl}-1-pyrrolidinecarboxylate

To a solution of 1,1-dimethylethyl (3S)-3-(aminomethyl)-1-pyrrolidinecarboxylate (1.15 g) in 50 mL dry DMSO was added DIEA (5.35 g) and 1-bromo-3-fluoro-2-ntirobenzene (5 g). The flask was purged with nitrogen and heated at 70° C. overnight. The reaction mixture was allowed to cool and was then diluted with diethyl ether, washed with brine and dried over sodium sulfate. The solvent was removed by evaporation and the crude product purified by silica gel column chromatography using petroleum ether/EtOAC to afford 6.5 g of the titled product.

### (b) (3-Bromo-2-nitrophenyl){[(3S)-1-(cyclopropylcarbonyl)-3-pyrrolidinyl]methyl}amine

1,1-Dimethylethyl (3S)-3-{[(3-bromo-2-nitrophenyl)amino]methyl}-1-pyrrolidinecarboxylate was dissolved in 50 mL 5 M HCl/1,4-dioxane and stirred at RT overnight, filtered and evaporated to dryness to yield the amine hydrochloride salt, which was dissolved in 15 mL dry DCM. TEA (5.5 g) was added and the reaction mixture was cooled to 0° C. Cyclopropylcarbonyl chloride (2.08 g) was added dropwise and the reaction was allowed to warm to RT and stirred for 3 days. The reaction mixture was washed with saturated brine and aqueous NH4Cl, dried over sodium sulfate and evaporated to dryness to afford the titled product, which was used without further purification.

### (c) (2-Amino-3-bromophenyl){[(3S)-1-(cyclopropylcarbonyl)-3-pyrrolidinyl]methyl}amine

(3-Bromo-2-nitrophenyl) {[(3S)-1-(cyclopropylcarbonyl)-3-pyrrolidinyl]methyl}amine (6.6 g) was dissolved in a mixture of 30 mL EtOAc, 15 mL MeOH and 15 mL DMF. To this was added SnCl2 (10 g). The flask was purged with nitrogen and the reaction mixture heated at reflux overnight. The reaction mixture was allowed to cool and diluted with EtOAc. Solid Na2CO3 was added and the reaction mixture was stirred at RT for 30 min. The reaction mixture was filtered, washed with saturated brine, dried over sodium sulfate and evaporated to dryness to afford the titled compound, which was used without further purification.

### (d) 4-Bromo-1-{[(3S)-1-(cyclopropylcarbonyl)-3-pyrrolidinyl]methyl}-2-(4′-fluoro-4-biphenylyl)-1H-benzimidazole

To a solution of (2-amino-3-bromophenyl){[(3S)-1-(cyclopropylcarbonyl)-3-pyrrolidinyl]methyl}amine (100 mg) in 3 mL DMF and 2 drops of water was added 4-(4′-fluorophenyl)benzaldehyde (60 mg) and oxone (120 mg,) and the reaction mixture was stirred at RT overnight. The reaction mixture was diluted with 2 mL MeOH, filtered and purified directly by preparative reverse phase HPLC to afford 124 mg of the titled compound (LCMS m/z 518.2, M+H).

### Example 30

### 4-Bromo-1-{[(3S)-1-(cyclopropylcarbonyl)-3-pyrrolidinyl]methyl}-2-[4′-(methyloxy)-4-biphenylyl]-1H-benzimidazole

The titled compound was prepared according to the procedure in Example 29(d), substituting 4-(4′-methoxyphenyl)benzaldehyde, to afford 127 mg of the desired product (LCMS m/z 530.2, M+H).

### Example 31

### 4-Bromo-1-{[(3S)-1-(cyclopropylcarbonyl)-3-pyrrolidinyl]methyl}-2-[4-(1H-indol-5-yl)phenyl]-1H-benzimidazole

The titled compound was prepared according to the procedure in Example 29(d), substituting 4-(1H-indol-5-yl)benzaldehyde, to afford 129 mg of the desired product (LCMS m/z 539.2, M+H).

### Example 32

### 5-[4-(4-Bromo-1-{[(3S)-1-(cyclopropylcarbonyl)-3-pyrrolidinyl]methyl}-1H-benzimidazol-2-yl)phenyl]-1H-indazole

The titled compound was prepared according to the procedure in Example 29(d), substituting 5-(4-formylphenyl)-1H-indazole, to afford 129 mg of the desired product (LCMS m/z 540.2).

### Example 33

### 2-(4-Biphenylyl)-4-bromo-1-{[(3S)-1-(cyclopropylcarbonyl)-3-pyrrolidinyl]methyl}-1H-benzimidazole

The titled compound was prepared according to the procedure in Example 29(d), substituting 4-biphenylcarbaldehyde, to afford 120 mg of the desired product (LCMS m/z 500.2, M+H).

### Example 34

### 4-Bromo-1-{[(3S)-1-(cyclopropylcarbonyl)-3-pyrrolidinyl]methyl}-2-[4-(1H-indol-6-yl)phenyl]-1H-benzimidazole

The titled compound was prepared according to the procedure in Example 29(d), substituting 4-(1H-Indol-6-yl)benzaldehyde, to afford 50 mg of the desired product (LCMS m/z 539.2, M+H).

### Example 35

### 6-[4-(4-Bromo-1-{[(3S)-1-(cyclopropylcarbonyl)-3-pyrrolidinyl]methyl}-1H-benzimidazol-2-yl)phenyl]-1H-indazole

The titled compound was prepared according to the procedure in Example 29(d), substituting 6-(4-formylphenyl)-1H-indazole, to afford 50 mg of the desired product (LCMS m/z 540.2, M+H).

### Example 36

### 2-[4-(1-Benzofuran-5-yl)phenyl]-4-bromo-1-{[(3S)-1-(cyclopropylcarbonyl)-3-pyrrolidinyl]methyl}-1H-benzimidazole

The titled compound was prepared according to the procedure in Example 29(d), starting with 111 mg of (2-amino-3-bromophenyl){[(3S)-1-(cyclopropylcarbonyl)-3-pyrrolidinyl]methyl}amine and substituting 4-(1-benzofuran-5-yl)benzaldehyde, to afford 93 mg of the desired product (LCMS m/z 540.2, M+H).

### Example 37

### 2-[4-(1-Benzofuran-5-yl)phenyl]-1-{[(3S)-1-(cyclopropylcarbonyl)-3-pyrrolidinyl]methyl}-5-(methyloxy)-1H-benzimidazole

### (a) 1,1-Dimethylethyl (3S)-3-({[4-(methyloxy)-2-nitrophenyl]amino}methyl)-1-pyrrolidinecarboxylate

Four drops of pyridine were added to 7 g 3-nitro-4-bromoanisole. To this was added 1,1-dimethylethyl (3S)-3-(aminomethyl)-1-pyrrolidinecarboxylate (60.6 g), K2CO3 (6.2 g) and CuI (283 mg) and the reaction mixture was stirred at 100° C. overnight. After cooling, the reaction mixture was diluted with EtOAc and the organic layer was collected, washed with water, and dried over sodium sulfate. Solvent was removed by evaporation and the crude product was purified by silica gel column chromatography using petroleum ether/EtOAc to afford the titled compound.

### (b) N-{[(3S)-1-(cyclopropylcarbonyl)-3-pyrrolidinyl]methyl}-4-(methyloxy)-2-nitroaniline

1,1-Dimethylethyl (3S)-3-({[4-(methyloxy)-2-nitrophenyl]amino}methyl)-1-pyrrolidinecarboxylate was treated with 4 N HCl/1,4-dioxane for 2 hr at RT. Removal of the solvent by evaporation gave the intermediate amine hydrochloride salt (3.9 g), which was dissolved in 40 mL DCM and 4.0 g DIEA and stirred at RT for 30 min. Cyclopropylcarbonyl chloride (1.7 g) was added dropwise and the reaction mixture stirred overnight at RT. The reaction mixture was treated with saturated aqueous NaHCO3 and the organic layer collected. The aqueous layer was washed with DCM and the combined organic layers were dried over sodium sulfate and evaporated to dryness. The crude product was purified by silica gel column chromatography using petroleum ether/EtOAc to afford the titled compound.

### (c) N¹-{[(3S)-1-(cyclopropylcarbonyl)-3-pyrrolidinyl]methyl}-4-(methyloxy)-1,2-benzenediamine

A solution of N-{[(3S)-1-(cyclopropylcarbonyl)-3-pyrrolidinyl]methyl}-4-(methyloxy)-2-nitro aniline in ethanol was added to a round bottom flask containing 10% Pd/C (0.1 eq) under nitrogen. The flask was evacuated and flushed with nitrogen three times and flushed with hydrogen twice. The reaction mixture was hydrogenated under a hydrogen atmosphere (balloon) for 1 hr at RT. The reaction mixture was then filtered through Celite and evaporated to dryness to yield the titled compound, which was used without further purification.

### (d) 2-[4-(1-Benzofuran-5-yl)phenyl]-1-{[(3S)-1-(cyclopropylcarbonyl)-3-pyrrolidinyl]methyl}-5-(methyloxy)-1H-benzimidazole

N1-{[(3S)-1-(cyclopropylcarbonyl)-3-pyrrolidinyl]methyl}-4-(methyloxy)-1,2-benzenediamine (70 mg) and (1-benzofuran-5-yl)benzaldehyde (54 mg) were dissolved in 4 mL DMF. To this was added oxone (97 mg) and the reaction was stirred at RT overnight. The reaction mixture was filtered and purified by reverse phase HPLC to afford 24 mg of the titled compound (LCMS m/z 492.4, M+H).

### Example 38

### 1-{[(3S)-1-(Cyclopropylcarbonyl)-3-pyrrolidinyl]methyl}-2-[4-(1H-indol-5-yl)phenyl]-5-(methyloxy)-1H-benzimidazole

The titled compound was prepared according to the procedure in Example 37(d), substituting 4-(1H-indol-5-yl)benzaldehyde, to afford 23 mg of the desired product (LCMS m/z 491.4, M+H).

### Example 39

### 1-{[(3S)-1-(Cyclopropylcarbonyl)-3-pyrrolidinyl]methyl}-2-[4-(1H-indol-6-yl)phenyl]-5-(methyloxy)-1H-benzimidazole

The titled compound was prepared according to the procedure in Example 37(d), substituting 4-(1H-indol-6-yl)benzaldehyde, to afford 20 mg of the desired product (LCMS m/z 491.4, M+H).

### Example 40

### 5-{4-[1-{[(3S)-1-(Cyclopropylcarbonyl)-3-pyrrolidinyl]methyl}-5-(methyloxy)-1H-benzimidazol-2-yl]phenyl}-1H-indazole

The titled compound was prepared according to the procedure in Example 37(d), substituting 5-(4-formylphenyl)-1H-indazole, to afford 20 mg of the desired product (LCMS m/z 492.4, M+H).

### Example 41

### 6-{4-[1-{[(3S)-1-(Cyclopropylcarbonyl)-3-pyrrolidinyl]methyl}-5-(methyloxy)-1H-benzimidazol-2-yl]phenyl}-1H-indazole

The titled compound was prepared according to the procedure in Example 37(d), substituting 6-(4-formylphenyl)-1H-indazole, to afford 26 mg of the desired product (LCMS m/z 492.4, M+H).

### Example 42

### 2-(4-Biphenylyl)-1-{[(3S)-1-(cyclopropylcarbonyl)-3-pyrrolidinyl]methyl}-5-(methyloxy)-1H-benzimidazole

The titled compound was prepared according to the procedure in Example 37(d), substituting 4-biphenylcarbaldehyde, to afford 30 mg of the desired product (LCMS m/z 452.4, M+H).

### Example 43

### 1-{[(3S)-1-(Cyclopropylcarbonyl)-3-pyrrolidinyl]methyl}-2-(4′-fluoro-4-biphenylyl)-5-(methyloxy)-1H-benzimidazole

The titled compound was prepared according to the procedure in Example 37(d), using 3 mL DMF and substituting 4-(4′-fluorophenyl)benzaldehyde, to afford 56 mg of the desired product (LCMS m/z 470.4, M+H).

### Example 44

### 1-{[(3S)-1-(Cyclopropylcarbonyl)-3-pyrrolidinyl]methyl}-5-(methyloxy)-2-[4′-(methyloxy)-4-biphenylyl]-1H-benzimidazole

The titled compound was prepared according to the procedure in Example 37(d), using 3 mL DMF and substituting 4-(4′-methoxyphenyl)benzaldehyde, to afford 58 mg of the desired product (LCMS m/z 482.4, M+H).

### Example 45

### 1-{[(3S)-1-(Cyclopropylcarbonyl)-3-pyrrolidinyl]methyl}-2-(4′-fluoro-4-biphenylyl)-5-methyl-1H-benzimidazole

### (a) 1,1-Dimethylethyl (3S)-3-{[(4-methyl-2-nitrophenyl)amino]methyl}-1-pyrrolidinecarboxylate

1-Fluoro-4-methyl-2-nitrobenzene (5 g), 1,1-dimethylethyl (3S)-3-(aminomethyl)-1-pyrrolidinecarboxylate (7.1 g) and DIEA (10.4 g) were dissolved in 30 mL DMSO and stirred at 80° C. overnight. The reaction mixture was allowed to cool and then was diluted with EtOAc. The organic layer was washed with water and saturated brine, dried over sodium sulfate and evaporated to dryness. The crude product was purified by silica gel column chromatography using petroleum ether/EtOAc to yield the titled compound.

### (b) N-{[(3S)-1-(cyclopropylcarbonyl)-3-pyrrolidinyl]methyl}-4-methyl-2-nitroaniline

1,1-Dimethylethyl (3S)-3-{[(4-methyl-2-nitrophenyl)amino]methyl}-1-pyrrolidinecarboxylate was treated with 50 mL 5 N HCl/MeOH for 4 hr at RT. Removal of the solvent by evaporation yielded the intermediate amine hydrochloride salt, which was dissolved in 100 mL DCM. DIEA (10.5 g) was added followed by cyclopropylcarbonyl chloride (3.11 geq). The reaction was stirred at RT overnight. The solvent was removed by evaporation and the residue was dissolved in EtOAc, washed with water and saturated brine, dried over sodium sulfate and evaporated to dryness to afford the titled compound, which was used without further purification.

### (c) N¹-{[(3S)-1-(Cyclopropylcarbonyl)-3-pyrrolidinyl]methyl}-4-methyl-1,2-benzenediamine

N-{[(3S)-1-(cyclopropylcarbonyl)-3-pyrrolidinyl]methyl}-4-methyl-2-nitroaniline (8 g) was dissolved in 500 mL EtOH and 10 mL MeOH. To this was added 10% Pd/C (1 g) and the reaction mixture was stirred under a hydrogen atmosphere overnight at RT. The reaction mixture was filtered and the solvent removed by evaporation to afford the titled compound, which was used without further purification.

### (d) 1-{[(3S)-1-(Cyclopropylcarbonyl)-3-pyrrolidinyl]methyl}-2-(4′-fluoro-4-biphenylyl)-5-methyl-1H-benzimidazole

N1-{[(3S)-1-(Cyclopropylcarbonyl)-3-pyrrolidinyl]methyl}-4-methyl-1,2-benzenediamine (120 mg) and 4-(4′-fluorophenyl)benzaldehyde (99 mg) were dissolved in 3 mL DMF. To this was added oxone (175 mg) and the reaction was stirred at RT overnight. The reaction mixture was filtered and purified by reverse phase HPLC, to afford 78 mg of the titled compound (LCMS m/z 454.2, M+H).

### Example 46

### 1-{[(3S)-1-(Cyclopropylcarbonyl)-3-pyrrolidinyl]methyl}-5-methyl-2-[4′-(methyloxy)-4-biphenylyl]-1H-benzimidazole

The titled compound was prepared according to the procedure in Example 45(d), substituting 4-(4′methoxyphenyl)benzaldehyde, to afford 75 mg of the desired product (LCMS m/z 466.2, M+H).

### Example 47

### 1-{[(3S)-1-(Cyclopropylcarbonyl)-3-pyrrolidinyl]methyl}-2-[4-(1H-indol-5-yl)phenyl]-5-methyl-1H-benzimidazole

The titled compound was prepared according to the procedure in Example 45(d), substituting 4-(1H-indol-5-yl)benzaldehyde, to afford 15 mg of the desired product (LCMS m/z 475.4, M+H).

### Example 48

### 1-{[(3S)-1-(Cyclopropylcarbonyl)-3-pyrrolidinyl]methyl}-2-[4-(1H-indol-6-yl)phenyl]-5-methyl-1H-benzimidazole

The titled compound was prepared according to the procedure in Example 45(d), substituting 4-(1H-indol-6-yl)benzaldehyde, to afford 15 mg of the desired product (LCMS m/z 475.3, M+H).

### Example 49

### 5-[4-(1-{[(3S)-1-(Cyclopropylcarbonyl)-3-pyrrolidinyl]methyl}-5-methyl-1H-benzimidazol-2-yl)phenyl]-1H-indazole

The titled compound was prepared according to the procedure in Example 45(d), substituting 5-(4-formylphenyl)-1H-indazole, to afford 50 mg of the desired product (LCMS m/z 476.4, M+H).

### Example 50

### 6-[4-(1-{[(3S)-1-(Cyclopropylcarbonyl)-3-pyrrolidinyl]methyl}-5-methyl-1H-benzimidazol-2-yl)phenyl]-1H-indazole

The titled compound was prepared according to the procedure in Example 45(d), substituting 6-(4-formylphenyl)-1H-indazole, to afford 50 mg of the desired product (LCMS m/z 476.4, M+H).

### Example 51

### 2-(4-Biphenylyl)-1-{[(3S)-1-(cyclopropylcarbonyl)-3-pyrrolidinyl]methyl}-5-methyl-1H-benzimidazole

The titled compound was prepared according to the procedure in Example 45(d), substituting 4-biphenylcarbaldehyde, to afford 80 mg of the desired product (LCMS m/z 436.0, M+H).

### Example 52

### 2-[4-(1-Benzofuran-5-yl)phenyl]-1-{[(3S)-1-(cyclopropylcarbonyl)-3-pyrrolidinyl]methyl}-5-methyl-1H-benzimidazole

The titled compound was prepared according to the procedure in Example 45(d), substituting (1-benzofuran-5-yl)benzaldehyde, to afford 67 mg of the desired product (LCMS m/z 476.3, M+H).

### Example 53

### 5-{4-[1-{[(3S)-1-(Cyclopropylcarbonyl)-3-pyrrolidinyl]methyl}-5-(trifluoromethyl)-1H-benzimidazol-2-yl]phenyl}-1H-indazole

### (a) 1,1-Dimethylethyl (3S)-3-({[2-nitro-4-(trifluoromethyl)phenyl]amino}methyl)-1-pyrrolidinecarboxylate

1-Fluoro-2-nitro-4-(trifluoromethyl)benzene (5 g) was dissolved in 50 mL DMSO. To this was added 1,1-dimethylethyl (3S)-3-(aminomethyl)-1-pyrrolidinecarboxylate (5.75 g) and DIEA (6.17 g) and the reaction mixture was heated to 80° C. and stirred overnight. After cooling, the reaction mixture was diluted with 50 mL water and extracted with EtOAc (2×150 mL). The combined extracts were dried over sodium sulfate and evaporated to dryness to afford the titled compound, which was used without further purification.

### (b) N-{[(3S)-1-(Cyclopropylcarbonyl)-3-pyrrolidinyl]methyl}-2-nitro-4-(trifluoromethyl)aniline

1,1-Dimethylethyl (3S)-3-({[2-nitro-4-(trifluoromethyl)phenyl]amino}methyl)-1-pyrrolidinecarboxylate was treated with 5 N HCl/MeOH at RT for 4 hr and then evaporated to dryness, to yield the intermediate amine hydrochloride salt, which was dissolved in 60 mL DCM. DIEA (12.88 g) was then added and the reaction mixture allowed to stir at RT for 30 min. Cyclopropylcarbonyl chloride (3.83 g) was then added dropwise and the reaction mixture allowed to stir at RT overnight. Saturated aqueous NaHCO3 was then added dropwise and the reaction mixture was extracted with EtOAc. The combined organic extracts were washed with water and saturated brine, dried over sodium sulfate and then evaporated to dryness to afford the title compound, which was used without further purification.

### (c) N¹-{[(3S)-1-(Cyclopropylcarbonyl)-3-pyrrolidinyl]methyl}-4-(trifluoromethyl)-1,2-benzenediamine

N-{[(3S)-1-(Cyclopropylcarbonyl)-3-pyrrolidinyl]methyl}-2-nitro-4-(trifluoromethyl)aniline (7.2 g) was dissolved in 70 mL MeOH. To this was added 10% Pd/C under argon, and the reaction was stirred under an atmosphere of hydrogen at RT overnight. The reaction mixture was filtered, dried over sodium sulfate, and evaporated to dryness to afford the titled compound, which was used without further purification.

### (d) 5-{4-[1-{[(3S)-1-(Cyclopropylcarbonyl)-3-pyrrolidinyl]methyl}-5-(trifluoromethyl)-1H-benzimidazol-2-yl]phenyl}-1H-indazole

N1-{[(3S)-1-(Cyclopropylcarbonyl)-3-pyrrolidinyl]methyl}-4-(trifluoromethyl)-1,2-benzenediamine (150 mg) was dissolved in 2 mL DMF. To this was added 5-(4-formylphenyl)-1H-indazole (105.7 mg) and oxone (183 mg) and the reaction mixture was stirred at RT overnight. The reaction mixture was diluted with 10 mL water and extracted with EtOAc (2×20 mL). The combined extracts were washed with saturated brine, dried over sodium sulfate and evaporated to dryness. The crude product was purified by preparative reverse phase HPLC, affording 107 mg of the titled compound (LCMS m/z 530.4, M+H).

### Example 54

### 1-{[(3S)-1-(Cyclopropylcarbonyl)-3-pyrrolidinyl]methyl}-2-[4-(1H-indol-6-yl)phenyl]-5-(trifluoromethyl)-1H-benzimidazole

The titled compound was prepared according to the procedure in Example 53(d), substituting 4-(1H-indol-6-yl)benzaldehyde, to afford 23 mg of the desired product (LCMS m/z 529.3, M+H).

### Example 55

### 6-{4-[1-{[(3S)-1-(Cyclopropylcarbonyl)-3-pyrrolidinyl]methyl}-5-(trifluoromethyl)-1H-benzimidazol-2-yl]phenyl}-1H-indazole

The titled compound was prepared according to the procedure in Example 53(d), substituting 6-(4-formylphenyl)-1H-indazole, to afford 100 mg of the desired product (LCMS m/z 530.4, M+H).

### Example 56

### 2-(4-biphenylyl)-1-{[(3S)-1-(Cyclopropylcarbonyl)-3-pyrrolidinyl]methyl}-5-(trifluoromethyl)-1H-benzimidazole

The titled compound was prepared according to the procedure in Example 53(d), substituting 4-biphenylcarbaldehyde, to afford 153 mg of the desired product (LCMS m/z 490.4, M+H).

### Example 57

### 2-[4-(1-Benzofuran-5-yl)phenyl]-1-{[(3S)-1-(cyclopropylcarbonyl)-3-pyrrolidinyl]methyl}-5-(trifluoromethyl)-1H-benzimidazole

The titled compound was prepared according to the procedure in Example 53(d), substituting (1-benzofuran-5-yl)benzaldehyde, to afford 191 mg of the desired product (LCMS m/z 530.4, M+H).

### Example 58

### 4′-[1-{[(3S)-1-(Cyclopropylcarbonyl)-3-pyrrolidinyl]methyl}-5-(trifluoromethyl)-1H-benzimidazol-2-yl]-3-biphenylol

The titled compound was prepared according to the procedure in Example 53(d), substituting 4-(3′-hydroxyhenyl)benzaldehyde, to afford 127 mg of the desired product (LCMS m/z 506.2, M+H).

### Example 59

### 1-{[(3S)-1-(Cyclopropylcarbonyl)-3-pyrrolidinyl]methyl}-2-(4′-fluoro-4-biphenylyl)-5-(trifluoromethyl)-1H-benzimidazole

The titled compound was prepared according to the procedure in Example 53(d), substituting 4-(4′-fluorophenyl)benzaldehyde, to afford 121.8 mg of the desired product (LCMS m/z 508.2, M+H).

### Example 60

### 1-{[(3S)-1-(Cyclopropylcarbonyl)-3-pyrrolidinyl]methyl}-2-[4′-(methyloxy)-4-biphenylyl]-5-(trffluoromethyl)-1H-benzimidazole

The titled compound was prepared according to the procedure in Example 53(d), substituting 4-(4′-methoxyphenyl)benzaldehyde, to afford 145.6 mg of the desired product (LCMS m/z 520.1, M+H).

### Example 61

### 2-(3′-Chloro-4-biphenylyl)-1-{[(3S)-1-(cyclopropylcarbonyl)-3-pyrrolidinyl]methyl}-5-(trifluoromethyl)-1H-benzimidazole

The titled compound was prepared according to the procedure in Example 53(d), substituting 4-(3′-chlorophenyl)benzaldehyde, to afford 119.8 mg of the desired product (LCMS m/z 524.3, M+H).

### Example 62

### 1-{[(3S)-1-(Cyclopropylcarbonyl)-3-pyrrolidinyl]methyl}-2-[4-(1H-indol-5-yl)phenyl]-5-(trifluoromethyl)-1H-benzimidazole

### (a) 1,1-Dimethylethyl (3S)-3-({[2-nitro-4-(trifluoromethyl)phenyl]amino}methyl)-1-pyrrolidinecarboxylate

1-Fluoro-2-nitro-4-(trifluoromethyl)benzene (1.55 g) was dissolved in 40 mL DMSO. To this was added 1,1-dimethylethyl (3S)-3-(aminomethyl)-1-pyrrolidinecarboxylate (2.07 g) and DIEA (4.06 mL) and the reaction mixture was stirred under nitrogen at 80° C. overnight. The reaction mixture was allowed to cool, diluted with 100 mL water and extracted with diethyl ether (3×100 mL). The combined ether extracts were washed with saturated brine (2×100 mL), dried over magnesium sulfate, and evaporated to dryness to afford the titled compound, which was used without further purification.

### (b) N-{[(3S)-1-(Cyclopropylcarbonyl)-3-pyrrolidinyl]methyl}-2-nitro-4-(trifluoromethyl)aniline

1,1-Dimethylethyl (3S)-3-({[2-nitro-4-(trifluoromethyl)phenyl]amino}methyl)-1-pyrrolidinecarboxylate was dissolved in 60 mL MeOH and 40 mL 4 NHCl/1,4-dioxane was added slowly with stirring. The reaction was stirred at RT for 1 hr. Solvent was removed by evaporation and the residue was dissolved in diethyl ether and evaporated to dryness to afford the intermediate amine hydrochloride salt, which was dissolved in 25 mL DCM. DIEA (3.88 mL) was added under nitrogen and the reaction mixture was allowed to stir at RT for 15 min. Cyclopropylcarbonyl chloride (0.852 g) in 25 mL DCM was then added dropwise and the reaction stirred at RT for 1 hr. The reaction was quenched with 50 mL saturated aqueous NaHCO3 and the organic layer was separated. The aqueous layer was washed once with DCM (50 mL) and the combined organic layers were dried over magnesium sulfate and evaporated to dryness. The crude product was purified by flash chromatography on silica gel using EtOAc/hexanes to afford the titled compound.

### (c) N¹-{[(3S)-1-(Cyclopropylcarbonyl)-3-pyrrolidinyl]methyl}-4-(trifluoromethyl)-1,2-benzenediamine

A dry, nitrogen-purged flask was charged with 10% Pd/C (0.35 g, 0.056 eq) and 25 mL EtOAc. N-{[(3S)-1-(Cyclopropylcarbonyl)-3-pyrrolidinyl]methyl}-2-nitro-4-(trifluoromethyl)aniline (2.1 g) was dissolved in 25 mL MeOH and slowly added to the flask containing the catalyst. The flask was evacuated and a hydrogen atmosphere was introduced by balloon. The reaction was stirred at RT for 2 hr. The reaction mixture was filtered through Celite and the Celite washed with a small amount of EtOAc. The combined filtrates were evaporated to dryness to afford the titled compound, which was used without further purification.

### (d) 2-(4-Bromophenyl)-1-{[(3S)-1-(cyclopropylcarbonyl)-3-pyrrolidinyl]methyl}-5-(trifluoromethyl)-1H-benzimidazole

N1-{[(3S)-1-(Cyclopropylcarbonyl)-3-pyrrolidinyl]methyl}-4-(trifluoromethyl)-1,2-benzenediamine (1.42 g) was dissolved in 30 mL n-butanol in a pressure vessel. 4-Bromobenzaldehyde (0.963 g) was added and the reaction mixture was stirred at 120° C. overnight. The reaction temperature was increased to 140° C. for 4 hr. The reaction mixture was allowed to cool and 10% Pd/C (0.462 g) was added in small portions. The pressure vessel was purged with nitrogen and heated to 80° C. for 1 hr. The reaction mixture was allowed to cool and then filtered through Celite. The Celite was washed several times with EtOAc and the combined filtrates were evaporated to dryness. The crude product was purified by flash chromatography on silica gel using 0-5% MeOH/DCM to afford the chemically pure product. An aliquot (1.3 g) was further purified by chiral SCF chromatography on a Daicel Chiralcel OJ-H column using 20% MeOH (containing 0.5% isopropylamine)/80% CO2 to afford the titled compound.

### (e) 1-{[(3S)-1-(Cyclopropylcarbonyl)-3-pyrrolidinyl]methyl}-2-[4-(1H-indol-5-yl)phenyl]-5-(trifluoromethyl)-1H-benzimidazole

2-(4-Bromophenyl)-1-{[(3S)-1-(cyclopropylcarbonyl)-3-pyrrolidinyl]methyl}-5-(trifluoromethyl)-1H-benzimidazole (200 mg) was dissolved in 3 mL 1,4-dioxane in a microwave reaction vial. To this was added 5-(4,4,5,5-tetramethyl-1,3,2-dioxaborolan-2-yl)-1H-indole (109 mg), potassium carbonate (0.609 mL of a 2 M solution) and PdCl2(dppf).CH2Cl2. The reaction vessel was purged with nitrogen, sealed and then heated in a microwave reaction to 100° C. for 3 hr. The reaction mixture was diluted with 50 mL water and the pH was adjusted to 7 with 1 N HCl. The reaction mixture was then extracted with DCM (3×50 mL) and the combined extracts were washed with saturated brine, dried over magnesium sulfate and evaporated to dryness. The crude product was purified by flash chromatography on silica gel using 0-5% MeOH/DCM followed by SCF purification on a Daicel Chiralcel OH-J column using MeOH (containing 0.5% isopropylamine)/CO2 to afford 115 mg of the titled compound (LCMS m/z 529.1, M+H).

### Example 63

### 5-Bromo-1-{[(3S)-1-(cyclopropylcarbonyl)-3-pyrrolidinyl]methyl}-6-fluoro-2-[4-(1H-indol-5-yl)phenyl]-1H-benzimidazole

### (a) 1,1-Dimethylethyl (3S)-3-{[(4-bromo-5-fluoro-2-nitrophenyl)amino]methyl}-1-pyrrolidinecarboxylate

1,1-Dimethylethyl (3S)-3-(aminomethyl)-1-pyrrolidinecarboxylate (1.0 g) and TEA (0.835 mL) were dissolved in 20 mL EtOH and stirred at RT for 20 min. 1-Bromo-2,4-difluoro-5-nitrobenzene (1.23 g) was the added and the reaction mixture was stirred at 80° C. overnight. After removal of the solvent the crude product was purified by flash chromatography on silica gel using EtOAc/hexanes to afford the titled compound.

### (b) (4-Bromo-5-fluoro-2-nitrophenyl){[(3S)-1-(cyclopropylcarbonyl)-3-pyrrolidinyl]methyl}amine

1,1-Dimethylethyl (3S)-3-{[(4-bromo-5-fluoro-2-nitrophenyl)amino]methyl}-1-pyrrolidinecarboxylate (1.65 g) was dissolved in about 3 mL of 1,4 dioxane and 4 N HCl/1,4-dioxane (20 mL) was then added via syringe. The reaction was stirred at RT for 1 hr and then evaporated to dryness to yield the intermediate amine hydrochloride. This was dissolved in 20 mL DCM along with cyclopropylcarbonyl chloride (0.71 g) and DIEA (3.95 mL) and the reaction stirred at RT for 2 hr. Solvent was removed by evaporation and the crude product purified by flash chromatography on silica gel using EtOAc followed by 2.5% MeOH/DCM to afford the titled compound.

### (c) 4-Bromo-N-{[(3S)-1-(cyclopropylcarbonyl)-3-pyrrolidinyl]methyl}-5-fluoro-1,2-benzenediamine

(4-Bromo-5-fluoro-2-nitrophenyl) {[(3S)-1-(cyclopropylcarbonyl)-3-pyrrolidinyl]methyl}amine (0.5 g) and SnCl2.2H2O (0.876 g) were dissolved in 10 mL EtOAc and stirred at 70° C. overnight. The reaction mixture was allowed to cool and then treated with 1 N NaOH and stirred at RT for 1 hr. The resulting suspension was filtered and the filtrate evaporated to dryness to afford the titled compound, which was used without further purification.

### (d) 5-Bromo-1-{[(3S)-1-(cyclopropylcarbonyl)-3-pyrrolidinyl]methyl}-6-fluoro-2-[4-(1H-indol-5-yl)phenyl]-1H-benzimidazole

4-Bromo-N-{[(3S)-1-(cyclopropylcarbonyl)-3-pyrrolidinyl]methyl}-5-fluoro-1,2-benzenediamine (50 mg) and 4-(1H-indol-5-yl)benzaldehyde (31 mg) were dissolved in 2 mL n-butanol and heated to 80° C. overnight. The reaction mixture was directly purified by preparative reverse phase HPLC to afford 18 mg of the titled compound (LCMS m/z 557.4, M+H).

### Example 64

### 2-(4-Biphenylyl)-5-bromo-1-{[(3S)-1-(cyclopropylcarbonyl)-3-pyrrolidinyl]methyl}-6-fluoro-1H-benzimidazole

The titled compound was prepared according to the procedure in Example 63(d), substituting 4-biphenylcarbaldehyde (LCMS m/z 518.2, M+H).

### Example 65

### 5-Bromo-1-{[(3S)-1-(cyclopropylcarbonyl)-3-pyrrolidinyl]methyl}-6-fluoro-2-[4′-(methyloxy)-4-biphenylyl]-1H-benzimidazole

The titled compound was prepared according to the procedure in Example 63(d), substituting 4-(4′-methoxyphenyl)benzaldehyde (LCMS m/z 548.1, M+H).

### Example 66

### 1-{[(3S)-1-(Cyclopropylcarbonyl)-3-pyrrolidinyl]methyl}-2-[4-(1H-indol-6-yl)phenyl]-6-(methyloxy)-1H-benzimidazole

### (a) 1,1-Dimethylethyl (3S)-3-({[5-(methyloxy)-2-nitrophenyl]amino}methyl)-1-pyrrolidinecarboxylate

3-Fluoro-4-nitroanisole (4 g), 1,1-dimethylethyl (3S)-3-(aminomethyl)-1-pyrrolidinecarboxylate (4.68 g) and triethylamine (6.52 mL) were dissolved in 50 mL ethanol and stirred at 70° C. for 18 hr. The solvent was removed by evaporation and the crude product purified by flash chromatography on silica gel using EtOAc/hexanes to afford the titled compound.

### (b) N-{[(3S)-1-(Cyclopropylcarbonyl)-3-pyrrolidinyl]methyl}-5-(methyloxy)-2-nitroaniline

1,1-Dimethylethyl (3S)-3-({[5-(methyloxy)-2-nitrophenyl]amino}methyl)-1-pyrrolidinecarboxylate (8.16 g) was treated with 175 mL 4 NHCl/1,4-dioxane under nitrogen at RT for 1 hr. Solvent was removed by evaporation to yield the amine hydrochloride salt (7.15 g), which was dissolved in 100 mL DCM. DIEA (11.56 mL) was added and the reaction mixture was stirred at RT for 5 min. Cyclopropylcarbonyl chloride (3.0 m) was added via syringe and the reaction stirred at RT for 90 min. The reaction mixture was partitioned between 50 mL water, 50 mL saturated NaHCO3 and 100 mL EtOAc. The organic layer was separated, washed with saturated brine, dried over magnesium sulfate and evaporated to dryness. The crude product was purified by flash chromatography on silica gel using EtOAc/hexanes to afford the titled compound.

### (c) N²-{[(3S)-1-(Cyclopropylcarbonyl)-3-pyrrolidinyl]methyl}-4-(methyloxy)-1,2-benzenediamine

N-{[(3S)-1-(cyclopropylcarbonyl)-3-pyrrolidinyl]methyl}-5-(methyloxy)-2-nitro aniline (6.9 g) was placed in a round bottom flask which was flushed with nitrogen 3 times. 10% Pd/C (2.184 g) was added followed by 2 mL EtOAc and then 300 mL EtOH. The flask again flushed with nitrogen 3 times and then hydrogenated under a hydrogen atmosphere (balloon) at RT for 3 hr. The reaction mixture was filtered through Celite and the Celite washed witn 100 mL EtOH. The combined filtrates were evaporated to dryness to afford the titled compound, which was used without further purification.

### (d) 1-{[(3S)-1-(Cyclopropylcarbonyl)-3-pyrrolidinyl]methyl}-2-[4-(1H-indol-6-yl)phenyl]-6-(methyloxy)-1H-benzimidazole

A pressure tube was charged with N2-{[(3S)-1-(cyclopropylcarbonyl)-3-pyrrolidinyl]methyl}-4-(methyloxy)-1,2-benzenediamine (1.29 g), 4-(1H-indol-6-yl)benzaldehyde (0.988 g) and 25 mL n-butanol and the reaction mixture was stirred at 115° C. for 66 hr. The reaction mixture was allowed to cool and solvent was removed by evaporation. The crude product was purified by flash chromatography on silica gel using EtOAc/hexanes, followed by preparative reverse phase HPLC, followed by preparative SFC using a Daicel Chiralcel OH-J column with MeOH (containing 0.5% isopropylamine)/CO2 to afford 520 mg of the titled compound (LCMS m/z 491.2, M+H).

### Example 67

### 4′-[1-{[(3S)-1-(cyclopropylcarbonyl)-3-pyrrolidinyl]methyl}-6-(methyloxy)-1H-benzimidazol-2-yl]-3-biphenylol

N2-{[(3S)-1-(cyclopropylcarbonyl)-3-pyrrolidinyl]methyl}-4-(methyloxy)-1,2-benzenediamine (120 mg) was dissolved in 3 mL DMF. To this was added 4-(3′-hydroxyphenyl)benzaldehyde (82 mg) and oxone (165.7 mg) and the reaction was stirred at RT overnight. The reaction mixture was filtered and then directly purified by preparative reverse phase HPLC to afford 27 mg of the titled compound (LCMS m/z 468.2, M+H).

### Example 68

### 1-{[(3S)-1-(Cyclopropylcarbonyl)-3-pyrrolidinyl]methyl}-2-(4′-fluoro-4-biphenylyl)-6-(methyloxy)-1H-benzimidazole

The titled compound was prepared according to the procedure in Example 67, substituting 4-(4′-fluorophenyl)benzaldehyde, to afford 54 mg of the desired product (LCMS m/z 470.2, M+H).

### Example 69

### 1-{[(3S)-1-(Cyclopropylcarbonyl)-3-pyrrolidinyl]methyl}-6-(methyloxy)-2-[4′-(methyloxy)-4-biphenylyl]-1H-benzimidazole

The titled compound was prepared according to the procedure in Example 67, substituting 4-(4′-methoxyphenyl)benzaldehyde, to afford 60 mg of the desired product. (LCMS m/z 482.1, M+H).

### Example 70

### 1-{[(3S)-1-(Cyclopropylcarbonyl)-3-pyrrolidinyl]methyl}-2-[4-(1H-indol-5-yl)phenyl]-6-(methyloxy)-1H-benzimidazole

The titled compound was prepared according to the procedure in Example 67, substituting 4-(1H-indol-5-yl)benzaldehyde, to afford 85 mg of the desired product (LCMS m/z 491.2, M+H).

### Example 71

### 5-{4-[1-{[(3S)-1-(Cyclopropylcarbonyl)-3-pyrrolidinyl]methyl}-6-(methyloxy)-1H-benzimidazol-2-yl]phenyl}-1H-indazole

The titled compound was prepared according to the procedure in Example 67, substituting 5-(4-formylphenyl)-1H-indazole, to afford 45 mg of the desired product (LCMS m/z 492.3, M+H).

### Example 72

### 6-{4-[1-{[(3S)-1-(Cyclopropylcarbonyl)-3-pyrrolidinyl]methyl}-6-(methyloxy)-1H-benzimidazol-2-yl]phenyl}-1H-indazole

The titled compound was prepared according to the procedure in Example 67, substituting 6-(4-formylphenyl)-1H-indazole, to afford 59 mg of the desired product (LCMS m/z 492.3, M+H).

### Example 73

### 2-(4-Biphenylyl)-1-{[(3S)-1-(cyclopropylcarbonyl)-3-pyrrolidinyl]methyl}-6-(methyloxy)-1H-benzimidazole

The titled compound was prepared according to the procedure in Example 67, substituting 4-biphenylcarbaldehyde, to afford 84 mg of the desired product (LCMS m/z 452.2, M+H).

### Example 74

### 2-[4-(1-Benzofuran-5-yl)phenyl]-1-{[(3S)-1-(cyclopropylcarbonyl)-3-pyrrolidinyl]methyl}-6-(methyloxy)-1H-benzimidazole

N2-{[(3S)-1-(cyclopropylcarbonyl)-3-pyrrolidinyl]methyl}-4-(methyloxy)-1,2-benzenediamine (150 mg) was dissolved in 2 mL DMF. To this was added (1-benzofuran-5-yl)benzaldehyde (115.2 mg) and oxone (207 mg) and the reaction was stirred at RT overnight. The reaction mixture was diluted with 10 mL water and extracted with EtOAc (2×20 mL). The combined organic extracts were washed with saturated brine, dried over sodium sulfate and evaporated to dryness. The crude product was purified by preparative reverse phase HPLC to afford 89 mg of the titled compound (LCMS m/z 492.4, M+H).

### Example 75

### 4′-(1-{[(3S)-1-(Cyclopropylcarbonyl)-3-pyrrolidinyl]methyl}-6-methyl-1H-benzimidazol-2-yl)-3-biphenylol

### (a) 1,1-Dimethylethyl (3S)-3-{[(5-methyl-2-nitrophenyl)amino]methyl}-1-pyrrolidinecarboxylate

To a solution of 7 g 4-fluoronitrotoluene in 70 mL DMSO was added 1,1-dimethylethyl (3S)-3-(aminomethyl)-1-pyrrolidinecarboxylate (11.75 g) and DIEA (8.74 g) and the reaction mixture was stirred at 70° C. overnight. The reaction mixture was allowed to cool and then diluted with 50 mL water. The reaction mixture was extracted twice with EtOAc and the combine extracts were washed with water and saturated brine, dried over sodium sulfate and evaporated to dryness to afford the titled compound, which was used without further purification.

### (b) N-{[(3S)-1-(Cyclopropylcarbonyl)-3-pyrrolidinyl]methyl}-5-methyl-2-nitroaniline

1,1-Dimethylethyl (3S)-3-{[(5-methyl-2-nitrophenyl)amino]methyl}-1-pyrrolidinecarboxylate (1.06 g) was treated with 10 mL 4 N HCl/1,4-dioxane at RT for 3 hr. The reaction mixture was evaporated to dryness to afford the amine hydrochloride salt, which was dissolved in 10 mL DCM. DIEA (1.16 g) was then added and the reaction mixture stirred for 30 min. Cyclopropylcarbonyl chloride (0.472 g) was then added and the reaction mixture stirred at RT for 48 hr. The reaction mixture was washed with saturated NaHCO3 and the aqueous wash extracted with DCM. The combined organic layers were washed with saturated brine, dried over sodium sulfate and evaporated to dryness. The crude product was purified by silica gel column chromatography using petroleum ether/EtOAc to afford 0.339 g of the titled compound.

### (c) N²-{[(3S)-1-(Cyclopropylcarbonyl)-3-pyrrolidinyl]methyl}-4-methyl-1,2-benzenediamine

N-{[(3S)-1-(cyclopropylcarbonyl)-3-pyrrolidinyl]methyl}-5-methyl-2-nitroaniline (0.339 g) was dissolved in 5 mL MeOH. To this was added SnCl2.2H2O (744.6 mg) and the reaction mixture was stirred at reflux overnight. After cooling, the reaction mixture was diluted with 50 mL water and the pH was adjusted to ˜10 with solid Na2CO3. The reaction mixture was extracted with EtOAc and the extracts were washed with water and saturated brine and then dried over sodium sulfate. Removal of the solvent by evaporation afforded the titled compound, which was used without further purification.

### (d) 4′-(1-{[(3S)-1-(Cyclopropylcarbonyl)-3-pyrrolidinyl]methyl}-6-methyl-1H-benzimidazol-2-yl)-3-biphenylol

N2-{[(3S)-1-(Cyclopropylcarbonyl)-3-pyrrolidinyl]methyl}-4-methyl-1,2-benzenediamine (117.5 mg) was dissolved in 5 ml DMF. To this was added 4-(3′-hydroxyphenyl)benzaldehyde (85 mg) and oxone (172 mg) and the reaction mixture was stirred at RT overnight. The reaction mixture was added dropwise to 20 mL of 1 N K2CO3 and 10 mL water with vigorous stirring. This mixture was then extracted with EtOAc and the organic extract washed with water and brine and then dried over sodium sulfate. The solvent was removed by evaporation and the crude product purified by preparative reverse phase HPLC to afford 50 mg of the titled compound (LCMS m/z 452.2, M+H).

### Example 76

### 1-{[(3S)-1-(Cyclopropylcarbonyl)-3-pyrrolidinyl]methyl}-2-(4′-fluoro-4-biphenylyl)-6-methyl-1H-benzimidazole

The titled compound was prepared according to the procedure in Example 75(d), substituting 4-(4′-fluorophenyl)benzaldehyde, to afford 110 mg of the desired product (LCMS m/z 454.2, M+H).

### Example 77

### 1-{[(3S)-1-(Cyclopropylcarbonyl)-3-pyrrolidinyl]methyl}-6-methyl-2-[4′-(methyloxy)-4-biphenylyl]-1H-benzimidazole

The titled compound was prepared according to the procedure in Example 75(d), substituting 4-(4′-methoxyphenyl)benzaldehyde, to afford 200 mg of the desired product (LCMS m/z 466.2, M+H).

### Example 78

### 1-{[(3S)-1-(Cyclopropylcarbonyl)-3-pyrrolidinyl]methyl}-2-[4-(1H-indol-5-yl)phenyl]-6-methyl-1H-benzimidazole

The titlec compound was prepared according to the procedure in Example 75(d), substituting 4-(1H-indol-5-yl)benzaldehyde, to afford 32 mg of the desired product (LCMS m/z 475.3, M+H).

### Example 79

### 1-{[(3S)-1-(Cyclopropylcarbonyl)-3-pyrrolidinyl]methyl}-2-[4-(1H-indol-6-yl)phenyl]-6-methyl-1H-benzimidazole

The titled compound was prepared according to the procedure in Example 75(d), substituting 4-(1H-indol-6-yl)benzaldehyde, to afford 22 mg of the desired product (LCMS m/z 475.3, M+H).

### Example 80

### 6-[4-(1-{[(3S)-1-(Cyclopropylcarbonyl)-3-pyrrolidinyl]methyl}-6-methyl-1H -benzimidazol-2-yl)phenyl]-1H-indazole

The titled compound was prepared according to the procedure in Example 75(d), substituting 6-(4-formylphenyl)-1H-indazole, to afford 51 mg of the desired product (LCMS m/z 476.2, M+H).

### Example 81

### 5-[4-(1-{[(3S)-1-(Cyclopropylcarbonyl)-3-pyrrolidinyl]methyl}-6-methyl-1H-benzimidazol-2-yl)phenyl]-1H-indazole

The titled compound was prepared according to the procedure in Example 75(d), substituting 5-(4-formylphenyl)-1H-indazole, to afford 51 mg of the desired product (LCMS m/z 476.2, M+H).

### Example 82

### 2-(4-Biphenylyl)-1-{[(3S)-1-(cyclopropylcarbonyl)-3-pyrrolidinyl]methyl}-6-methyl-1H-benzimidazole

The titled compound was prepared according to the procedure in Example 75(d), substituting 4-biphenylcarbaldehyde, to afford 69 mg of the desired product (LCMS m/z 436.2, M+H).

### Example 83

### 2-[4-(1-Benzofuran-5-yl)phenyl]-1-{[(3S)-1-(cyclopropylcarbonyl)-3-pyrrolidinyl]methyl}-6-(methyl)-1H-benzimidazole

N2-{[(3S)-1-(Cyclopropylcarbonyl)-3-pyrrolidinyl]methyl}-4-methyl-1,2-benzenediamine (150 mg) was dissolved in 2 mL DMF. To this was added (1-benzofuran-5-yl)benzaldehyde (122.0 mg) and oxone (219.2 mg) and the reaction was stirred at RT overnight. The reaction mixture was diluted with 10 mL water and extracted with EtOAc (2×20 mL). The combined organic extracts were washed with brine, dried over sodium sulfate and evaporated to dryness. The crude product was purified by preparative reverse phase HPLC to afford 128 mg of the titled compound (LCMS m/z 476.4, M+H).

### Example 84

### 4′-[1-{[(3S)-1-(Cyclopropylcarbonyl)-3-pyrrolidinyl]methyl}-6-(trifluoromethyl)-1H-benzimidazol-2-yl]-3-biphenylol

### (a) 1,1-Dimethylethyl (3S)-3-({[2-nitro-5-(trifluoromethyl)phenyl]amino}methyl)-1-pyrrolidinecarboxylate

2-Chloro-1-nitro-4-(trifluoromethyl)benzene (6.75 g) and 1,1-dimethylethyl (3S)-3-(aminomethyl)-1-pyrrolidinecarboxylate (6 g) were dissolved in 50 mL DMSO. To this was added DIEA (5.42 g) and the reaction mixture was flushed with nitrogen and stirred at 90° C. overnight. The reaction mixture was allowed to cool, diluted with brine and extracted with methyl t-butyl ether. The organic extracts were dried and the solvent evaporated. The crude product was purified by silica gel column chromatography to afford the titled compound.

### (b) N-{[(3S)-1-(Cyclopropylcarbonyl)-3-pyrrolidinyl]methyl}-2-nitro-5-(trffluoromethyl)aniline

To a solution of 1,1-dimethylethyl (3S)-3-({[2-nitro-5-(trifluoromethyl)phenyl]amino}methyl)-1-pyrrolidinecarboxylate (6.3 g) in methanol was added 50 mL 5 N HCl/MeOH and the reaction mixture was stirred at RT overnight. The reaction mixture was evaporated to dryness to yield the amine hydrochloride salt, which was dissolved in 30 mL dry DCM and cooled to 0° C. Cyclopropylcarbonxyl chloride (1.62 g) was added and the reaction mixture was allowed to warm to RT and stirred overnight. The reaction mixture was diluted with EtOAc, washed with brine and saturated NH4Cl, dried and concentrated to afford the titled compound, which was used without further purification.

### (c) N²-{[(3S)-1-(Cyclopropylcarbonyl)-3-pyrrolidinyl]methyl}-4-(trifluoromethyl)-1,2-benzenediamine

To a suspension of 100 mg 10% Pd/C in 5 mL EtOH was added N-{[(3S)-1-(cyclopropylcarbonyl)-3-pyrrolidinyl]methyl}-2-nitro-5-(trifluoromethyl)aniline (357 mg). The reaction mixture was stirred under a hydrogen atmosphere for 3 hr at RT. The reaction mixture was filtered and the solvent removed by evaporation to afford the titled compound, which was used without further purification.

### (d) 4′-[1-{[(3S)-1-(Cyclopropylcarbonyl)-3-pyrrolidinyl]methyl}-6-(trifluoromethyl)-1H-benzimidazol-2-yl]-3-biphenylol

N2-{[(3S)-1-(Cyclopropylcarbonyl)-3-pyrrolidinyl]methyl}-4-(trifluoromethyl)-1,2-benzenediamine (100 mg) was dissolved in 3 mL DMF and 2 drops of water. To this was added 4-(3′-hydroxyphenyl)benzaldehyde (61 mg) and oxone (122 mg) and the reaction mixture was stirred overnight at RT. The reaction mixture was filtered and directly purified by preparative reverse phase HPLC to afford 66 mg of the titled compound (LCMS m/z 506.1, M+H).

### Example 85

### 1-{[(3S)-1-(Cyclopropylcarbonyl)-3-pyrrolidinyl]methyl}-2-(4′-fluoro-4-biphenylyl)-6-(trifluoromethyl)-1H-benzimidazole

The titled compound was prepared according to the procedure in Example 84(d), substituting 4-(4′-fluorophenyl)benzaldehyde, to afford 50 mg of the desired product (LCMS m/z 508.1, M+H).

### Example 86

### 1-{[(3S)-1-(Cyclopropylcarbonyl)-3-pyrrolidinyl]methyl}-2-[4′-(methyloxy)-4-biphenylyl]-6-(trifluoromethyl)-1H-benzimidazole

The titled compound was prepared according to the procedure in Example 84(d), substituting 4(4′-methoxyphenyl)benzaldehyde to afford 48 mg of the desired product (LCMS m/z 520.1, M+H).

### Example 87

### 1-{[(3S)-1-(Cyclopropylcarbonyl)-3-pyrrolidinyl]methyl}-2-[4-(1H-indol-5-yl)phenyl]-6-(trifluoromethyl)-1H-benzimidazole

The titled compound was prepared according to the procedure in Example 84(d), substituting 4-(1H-indol-5-yl)benzaldehyde, to afford 80 mg of the desired product (LCMS m/z 529.4, M+H).

### Example 88

### 1-{[(3S)-1-(Cyclopropylcarbonyl)-3-pyrrolidinyl]methyl}-2-[4-(1H-indol-6-yl)phenyl]-6-(trifluoromethyl)-1H-benzimidazole

The titled compound was prepared according to the procedure in Example 84(d), substituting 4-(1H-indol-6-yl)benzaldehyde, to afford 85 mg of the desired product (LCMS m/z 529.4, M+H).

### Alternate Procedure

### (a) N-{[(3S)-1-(Cyclopropylcarbonyl)-3-pyrrolidinyl]methyl}-2-nitro-5-(trifluoromethyl)aniline

A solution of 2-chloro-1-nitro-4-(trifluoromethyl)benzene (20.7 g) in toluene (300 mL) was treated with {[(3S)-1-(cyclopropylcarbonyl)-3-pyrrolidinyl]methyl}amine (16.1 g), BINAP (6.1 g), palladium(II) acetate (1.02 g), and cesium carbonate (47.2 g). With stirring the reaction was purged with nitrogen, sealed and heated to 100° C. over the weekend. The reaction was then diluted with water (500 mL) and was extracted with EtOAc (3×500 mL). The organic phase was washed with brine (500 mL), dried over sodium sulfate, filtered and concentrated in vacuo. The resulting brown material was purified by flash chromatography on silica gel using EtOAc/hexanes to afford the titled compound as a yellow solid (29.9 g, 87%). MS (ES)+ m/e 358.4 [M+H]+.

### (b) N²-{[(3S)-1-(cyclopropylcarbonyl)-3-pyrrolidinyl]methyl}-4-(trifluoromethyl)-1,2-benzenediamine

A suspension of 10% palladium on carbon (8.93 g) in EtOAc (50 mL) was treated with N-{[(3S)-1-(cyclopropylcarbonyl)-3-pyrrolidinyl]methyl}-2-nitro-5-(trifluoromethyl)aniline (29.9 g) in EtOH (50.0 mL) and under a hydrogen atmosphere (balloon). After 3 hours of stirring at room temperature, the reaction mixture was evacuated and backfilled with nitrogen. The mixture was filtered through Celite and concentrated in vacuo to afford the crude titled compound as a brown solid (25.8 grams, 94%), which was used without further purification. MS (ES)+ m/e 328.2 [M+H]+.

### (c) 2-(4-Bromophenyl)-1-{[(3S)-1-(cyclopropylcarbonyl)-3-pyrrolidinyl]methyl}-6-(trifluoromethyl)-1H-benzimidazole

To a solution of N²-{[(3S)-1-(cyclopropylcarbonyl)-3-pyrrolidinyl]methyl}-4-(trifluoromethyl)-1,2-benzenediamine (25.8 g) in 1-butanol (450 mL) was added 4-bromobenzaldehyde (16.05 g). The reaction mixture was for stirred 3 hours at 115° C. while a stream of air was slowly bubbled through the solution. The reaction mixture was allowed to cool and was concentrated in vacuo. The resulting yellow material was purified by flash chromatography on silica gel using MeOH/DCM to afford the titled compound as a yellow solid (21.5 g, 55%). MS (ES)+ m/e 492/424 [M+H]+.

### (d) 1-{[(3S)-1-(Cyclopropylcarbonyl)-3-pyrrolidinyl]methyl}-2-[4-(1H-indol-6-yl)phenyl]-6-(trifluoromethyl)-1H-benzimidazole

A solution of 2-(4-bromophenyl)-1-{[(3S)-1-(cyclopropylcarbonyl)-3-pyrrolidinyl]methyl}-6-(trifluoromethyl)-1H-benzimidazole (1.02 g) in 1,4-dioxane (15 mL) was treated with 1H-indol-6-ylboronic acid (0.348 g), aqueous potassium carbonate (2 M, 2.07 mL), and PdC12(dppf).CH2Cl2 (0.08 g). The reaction was purged with nitrogen, sealed and heated to 100° C. with stirring for one hour. The reaction solution was then cooled to room temperature and was diluted with water (50 mL). The aqueous solution was acidified to pH˜7 using 1N HCl (aq) and was extracted using CH2Cl2. The organic phase was dried over magnesium sulfate, filtered and was concentrated in vacuo to dryness.

Purification of the brown residue by flash chromatography on silica gel using MeOH/DCM afforded the titled compound as a beige solid (970 mg, 35%). MS (ES)+ m/e 529.2 [M+H]+.

### Example 89

### 5-{4-[1-{[(3S)-1-(cyclopropylcarbonyl)-3-pyrrolidinyl]methyl}-6-(trifluoromethyl)-1H-benzimidazol-2-yl]phenyl}-1H-indazole

The titled compound was prepared according to the procedure in Example 84(d), substituting 5-(4-formylphenyl)-1H-indazole, to afford 90 mg of the desired product (LCMS m/z 530.4, M+H).

### Example 90

### 6-{4-[1-{[(3S)-1-(cyclopropylcarbonyl)-3-pyrrolidinyl]methyl}-6-(trifluoromethyl)-1H-benzimidazol-2-yl]phenyl}-1H-indazole

The titled compound was prepared according to the procedure in Example 84(d), substituting 6-(4-formylphenyl)-1H-indazole, to afford 90 mg of the desired product (LCMS m/z 530.4, M+H).

### Example 91

### 2-(4-biphenylyl)-1-{[(3S)-1-(cyclopropylcarbonyl)-3-pyrrolidinyl]methyl}-6-(trifluoromethyl)-1H-benzimidazole

The titled compound was prepared according to the procedure in Example 84(d), substituting 4-biphenylcarbaldehyde, to afford 110 mg of the desired product (LCMS m/z 490.4, M+H).

### Example 92

### 2-[4-(1-benzofuran-5-yl)phenyl]-1-{[(3S)-1-(cyclopropylcarbonyl)-3-pyrrolidinyl]methyl}-6-(trifluoromethyl)-1H-benzimidazole

N2-{[(3S)-1-(Cyclopropylcarbonyl)-3-pyrrolidinyl]methyl}-4-(trifluoromethyl)-1,2-benzenediamine (108 mg) was dissolved in 3 mL DMF. To this was added (1-benzofuran-5-yl)benzaldehyde (80 mg) and oxone (144 mg) and the reaction mixture was stirred overnight at RT. The reaction mixture was directly purified by preparative reverse phase HPLC to afford 61 mg of the desired product (LCMS m/z 530.3, M+H).

### Example 93

### 2-(4-Biphenylyl)-6-bromo-1-{[(3S)-1-(cyclopropylcarbonyl)-3-pyrrolidinyl]methyl}-1H-benzimidazole

### (a) 1,1-Dimethylethyl (3S)-3-{[(5-bromo-2-nitrophenyl)amino]methyl}-1-pyrrolidinecarboxylate

4-Bromo-2-fluoro-1-nitrobenzene (5 g), 1,1-dimethylethyl (3S)-3-(aminomethyl)-1-pyrrolidinecarboxylate (5 g) and TEA (4.1 g) were dissolved in 50 mL DMSO and stirred at 70° C. overnight. The reaction mixture was diluted with EtOAc, washed with water and brine, dried over sodium sulfate and evaporated to dryness to afford the titled compound.

### (b) 5-Bromo-2-nitrophenyl){[(3S)-1-(cyclopropylcarbonyl)-3-pyrrolidinyl]methyl}amine

1,1-Dimethylethyl (3S)-3-{[(5-bromo-2-nitrophenyl)amino]methyl}-1-pyrrolidinecarboxylate (9.4 g) was dissolved in 100 mL MeOH. To this was added 250 mL 5 N HCl/MeOH and the reaction mixture was stirred at RT for 3 hr. Evaporation of the solvent gave 7.8 g of the amine hydrochloride salt, which was suspended in 100 mL DCM. TEA (7.1 g) was added and the reaction mixture was stirred for 15 min. The reaction mixture was then cooled to 0° C. and cyclopropylcarbonyl chloride (2.67 g) was slowly added. The reaction was allowed to warm to RT and stirred overnight. The reaction mixture was washed with water, brine and saturated aqueous NaHCO3, dried over sodium sulfate and evaporated to dryness to afford the titled compound, which was used without further purification.

### (c) 2-Amino-5-bromophenyl){[(3S)-1-(cyclopropylcarbonyl)-3-pyrrolidinyl]methyl}amine

5-Bromo-2-nitrophenyl){[(3S)-1-(cyclopropylcarbonyl)-3-pyrrolidinyl]methyl}amine (8 g) and SnCl2.2H2O (17.2 g) in 200 mL EtOAc were refluxed overnight. The reaction mixture was then cooled to 0° C. and 14 eq. of saturated aqueous NaOH was added and the reaction mixture stirred for 1 hr. The organic layer was separated, dried over sodium sulfate and evaporated to dryness to afford the titled compound, which was used without further purification.

### (d) 2-(4-Biphenylyl)-6-bromo-1-{[(3S)-1-(cyclopropylcarbonyl)-3-pyrrolidinyl]methyl}-1H-benzimidazole

2-Amino-5-bromophenyl) {[(3S)-1-(cyclopropylcarbonyl)-3-pyrrolidinyl]methyl}amine (100 mg) and 4-biphenylcarbaldehyde (53.8 mg) were dissolved in 5 mL DMF. To this was added oxone (118 mg) and the reaction mixture was stirred overnight at RT. The reaction mixture was filtered and directly purified by preparative reverse phase HPLC to afford 135 mg of the titled compound

### Example 94

### 4′-(B-bromo-1-{[(3S)-1-(cyclopropylcarbonyl)-3-pyrrolidinyl]methyl}-1H-benzimidazol-2-yl)-3-biphenylol

The titled compound was prepared according to the procedure in Example 93(d), substituting 4-(3′-hydroxyphenyl)benzaldehyde, to afford 91 mg of the desired product

### Example 95

### 6-Bromo-1-{[(3S)-1-(cyclopropylcarbonyl)-3-pyrrolidinyl]methyl}-2-(4′-fluoro-4-biphenylyl)-1H-benzimidazole

2-Amino-5-bromophenyl) {[(3S)-1-(cyclopropylcarbonyl)-3-pyrrolidinyl]methyl}amine (120 mg) and 4-(4′-fluorophenyl)benzaldehyde (71 mg) were dissolved in 5 mL DMF. To this was added oxone (141 mg) and the reaction mixture was stirred overnight at RT. The reaction mixture was filtered and directly purified by preparative reverse phase HPLC to afford 97 mg of the titled compound

### Example 96

### 6-Bromo-1-{[(3S)-1-(cyclopropylcarbonyl)-3-pyrrolidinyl]methyl}-2-[4′-(methyloxy)-4-biphenylyl]-1H-benzimidazole

The titled compound was prepared according the to procedure in Example 95, substituting 4-(4′-methoxyphenyl)benzaldehyde, to afford 74 mg of the desired product

### Example 97

### 6-Bromo-1-{[(3S)-1-(cyclopropylcarbonyl)-3-pyrrolidinyl]methyl}-2-[4-(1H-indol-5-yl)phenyl]-1H-benzimidazole

2-Amino-5-bromophenyl) {[(3S)-1-(cyclopropylcarbonyl)-3-pyrrolidinyl]methyl}amine (120 mg) and 4-(1H-indol-5-yl)benzaldehyde (78 mg) were dissolved in 4 mL DMF. To this was added oxone (141 mg) and the reaction mixture was stirred overnight at RT. The reaction mixture was filtered and directly purified by preparative reverse phase HPLC to afford 40 mg of the titled compound

### Example 98

### 6-Bromo-1-{[(3S)-1-(cyclopropylcarbonyl)-3-pyrrolidinyl]methyl}-2-[4-(1H-indol-6-yl)phenyl]-1H-benzimidazole

The titled compound was prepared according to the procedure in Example 97, substituting 4-(1H-indol-6-yl)benzaldehyde, to afford 30 mg of the desired product

### Example 99

### 5-[4-(6-Bromo-1-{[(3S)-1-(cyclopropylcarbonyl)-3-pyrrolidinyl]methyl}-1H-benzimidazol-2-yl)phenyl]-1H-indazole

The titled compound was prepared according to the procedure in Example 97, substituting 5-(4-formylphenyl)-1H-indazole, to afford 40 mg of the desired product

### Example 100

### 6-[4-(B-bromo-1-{[(3S)-1-(cyclopropylcarbonyl)-3-pyrrolidinyl]methyl}-1H-benzimidazol-2-yl)phenyl]-1H-indazole

The titled compound was prepared according to the procedure in Example 96, substituting 6-(4-formylphenyl)-1H-indazole, to afford 50 mg of the desired product

### Example 101

### 2-[4-(1-Benzofuran-5-yl)phenyl]-6-bromo-1-{[(3S)-1-(cyclopropylcarbonyl)-3-pyrrolidinyl]methyl}-1H-benzimidazole

2-Amino-5-bromophenyl) {[(3S)-1-(cyclopropylcarbonyl)-3-pyrrolidinyl]methyl}amine (120 mg) and (1-benzofuran-5-yl)benzaldehyde (79 mg) were dissolved in 4 mL DMF. To this was added oxone (141 mg) and the reaction mixture was stirred overnight at RT. The reaction mixture was filtered and directly purified by preparative reverse phase HPLC to afford 50 mg of the titled compound (LCMS m/z 540.2, M+H).

### Example 102

### 2-(4-Biphenylyl)-1-{[(3S)-1-(cyclopropylcarbonyl)-3-pyrrolidinyl]methyl}-N-methyl-1H-benzimidazole-6-carboxamide

### (a) Methyl 3-fluoro-4-nitrobenzoate

3-Fluoro-4-nitrobenzoic acid (3.42 g) was dissolved in 40 mL MeOH. To this was added 300 uL thionyl chloride, and the reaction mixture was stirred at 70° C. overnight. The reaction mixture was evaporated and the residue dissolved in EtOAc, washed with aqueous NaHCO3, and dried over sodium sulfate. Removal of solvent by evaporation afforded the titled compound, which was used without further purification.

### (b) 1,1-Dimethylethyl (3S)-3-[([5-[(methyloxy)carbonyl]-2-nitrophenyl]amino)methyl]-1-pyrrolidinecarboxylate

Methyl 3-fluoro-4-nitrobenzoate (3.1 g), 1,1-dimethylethyl (3S)-3-(aminomethyl)-1-pyrrolidinecarboxylate (2.19 g) and TEA (1.6 mL) were dissolved in 11 mL acetonitrile and stirred at 70° C. for 3 hrs. Reaction was incomplete so an additional aliquot of 1,1-dimethylethyl (3S)-3-(aminomethyl)-1-pyrrolidinecarboxylate (0.23 g) was added and the reaction was continued for 2 hr. The reaction mixture was allowed to cool and then filtered through an 8×10 cm plug of silica gel, eluting the silica gel with EtOAc. The filtrate was evaporated to dryness to afford the titled compound, which was used without further purification.

### (c) 1,1-Dimethylethyl (3S)-3-[({2-amino-5-[(methyloxy)carbonyl]phenyl}amino)methyl]-1-pyrrolidinecarboxylate

1,1-Dimethylethyl (3S)-3-[({5-[(methyloxy)carbonyl]-2-nitrophenyl}amino)methyl]-1-pyrrolidinecarboxylate (4.2 g) was dissolved in 30 mL EtOAc in a Parr shaker bottle. 10% Pd/C was added in 3 mL HOAc and the reaction mixture hydrogenated at 50 psi of hydrogen gas over night. The reaction mixture was filtered through a 6×10 cm plug of basic alumina, eluting with 750 mL EtOAc. The filtrate was evaporated to dryness to afford the titled compound, which was used without further purification.

### (d) Methyl 2-(4-bromophenyl)-1-[((3S)-1-{[(1,1-dimethylethyl)oxy]carbonyl}-3-pyrrolidinyl)methyl]-1H-benzimidazole-6-carboxylate

1,1-Dimethylethyl (3S)-3-[({2-amino-5-[(methyloxy)carbonyl]phenyl}amino)methyl]-1-pyrrolidinecarboxylate (3.81 g) and 4-bromobenzaldehyde (2.12 g) were dissolved in 20 mL n-butanol and stirred at 80° C. for 12 hr. The reaction mixture was allowed to cool and then evaporated to dryness. The residue was dissolved in EtOAc, washed with water, dried over magnesium sulfate and evaporated to dryness. The residue was dissolved in DCM and loaded onto a 10×10 cm silica gel plug. Elution with 400 mL DCM removed unreacted aldehyde. The product was eluted with 1:1 DCM/EtOAc and the eluate evaporated to dryness to afford the titled product.

### (e) Methyl 2-(4-bromophenyl)-1-{[(3S)-1-(cyclopropylcarbonyl)-3-pyrrolidinyl]methyl}-1H-benzimidazole-6-carboxylate

Methyl 2-(4-bromophenyl)-1-[((3S)-1-{[(1,1-dimethylethyl)oxy]carbonyl}-3-pyrrolidinyl)methyl]-1H-benzimidazole-6-carboxylate (5.14 g) was dissolved in 10 mL DCM, cooled to 0° C. and 10 mL TFA was added. After 15 min. the reaction mixture was evaporated to dryness. The residue was dissolved in EtOAc, applied to a 2×6 cm plug of basic alumina and eluted with EtOAc. The UV-active fractions were combined and evaporated to dryness. The residue was dissolved in ˜30 mL DCM and TEA (1.67 mL) and cyclopropylcarbonyl chloride (1.09 mL) were added. The reaction mixture was stirred for 20 min at RT and then quenched by the addition of 5 mL MeOH. The reaction mixture was washed with water (2×65 mL) and the water washes back extracted with DCM. The combined organic layers were dried over sodium sulfate and evaporated to dryness. The crude product was purified by flash chromatography on silica gel using EtOAc/DCM to afford the titled compound.

### (f) Methyl 2-(4-biphenylyl)-1-{[(3S)-1-(cyclopropylcarbonyl)-3-pyrrolidinyl]methyl}-1H-benzimidazole-6-carboxylate

Methyl 2-(4-bromophenyl)-1-{[(3S)-1-(cyclopropylcarbonyl)-3-pyrrolidinyl]methyl}-1H-benzimidazole-6-carboxylate (200 mg) was dissolved in 4 mL DMF in a reaction vial and sparged with nitrogen. Pd(PPh3)4 (24 mg) was added and the reaction mixture was stirred for 10 min. Phenylboronic acid (24 mg) and Na2CO3 (76 mg in 500 uL water) were then added and the vial was then purged again with nitrogen, capped and stirred at 80° C. overnight. The reaction mixture was allowed to cool, filtered and purified directly by preparative reverse phase HPLC. The appropriate fractions were combined, diluted with brine and extracted with EtOAc. The combined EtOAc extracts were dried over sodium sulfate and evaporated to dryness to afford the titled compound.

### (g) 2-(4-Biphenylyl)-1-{[(3S)-1-(cyclopropylcarbonyl)-3-pyrrolidinyl]methyl}-N-methyl-1H-benzimidazole-6-carboxamide

2 M Methylamine/MeOH was dried by passing it over a neutral alumina column. Methyl 2-(4-bromophenyl)-1-{[1-(cyclopropylcarbonyl)-3-pyrrolidinyl]methyl}-1H-benzimidazole-6-carboxylate (187 mg) was dissolved in 4 mL MeOH. To this was added 25 mL dried 2 M methylamine/MeOH and 4 mg sodium cyanide. The reaction was heated to 90° C. for 60 min, then allowed to cool and evaporated to dryness. The crude product was purified by preparative reverse phase HPLC to afford the titled compound (LCMS m/z 479.4, M+H).

### Example 103

### 1-{[(3S)-1-(cyclopropylcarbonyl)-3-pyrrolidinyl]methyl}-2-(4′-fluoro-4-biphenylyl)-N-methyl-1H-benzimidazole-6-carboxamide

### (a) Methyl 1-{[(3S)-1-(cyclopropylcarbonyl)-3-pyrrolidinyl]methyl}-2-(4′-fluoro-4-biphenylyl)-1H-benzimidazole-6-carboxylate

Following the procedure in Example 102(f), methyl 2-(4-bromophenyl)-1-{[(3S)-1-(cyclopropylcarbonyl)-3-pyrrolidinyl]methyl}-1H-benzimidazole-6-carboxylate (202 mg) was reacted with Pd(PPh3)4 (24 mg), Na2CO3 (62 mg in 500 uL water) and 4-fluorophenylboronic acid (82 mg) to afford the titled compound.

### (b) 1-{[(3S)-1-(Cyclopropylcarbonyl)-3-pyrrolidinyl]methyl}-2-(4′-fluoro-4-biphenylyl)-1H-benzimidazole-6-carboxylic acid

Methyl 1-{[(3S)-1-(cyclopropylcarbonyl)-3-pyrrolidinyl]methyl}-2-(4′-fluoro-4-biphenylyl)-1H-benzimidazole-6-carboxylate was dissolved in THF and treated with 7 eq. LiOH in water at RT overnight. The reaction mixture was acidified, diluted with brine and extracted with EtOAc. A precipitate developed which was filtered off. The EtOAc layer was separated, recombined with the precipitate and evaporated to dryness to afford the titled compound, which was used without further purification.

### (c) 1-{[(3S)-1-(cyclopropylcarbonyl)-3-pyrrolidinyl]methyl}-2-(4′-fluoro-4-biphenylyl)-N-methyl-1H-benzimidazole-6-carboxamide

1-{[(3S)-1-(Cyclopropylcarbonyl)-3-pyrrolidinyl]methyl}-2-(4′-fluoro-4-biphenylyl)-1H-benzimidazole-6-carboxylic acid (229 mg), HATU (216 mg), methylamine (2 M in THF, 1.19 mL) and TEA (80 uL) were dissolved in 4 mL DMF and stirred at RT for 1 hr. The reaction mixture was filtered and directly purified by preparative reverse phase HPLC. The appropriate fractions were combined, diluted with brine and extracted with EtOAc three times. The combined EtOAc extracts were dried over magnesium sulfate, evaporated, and the residue dried under vacuum to afford 109 mg of the titled compound (LCMS m/z 497.2, M+H).

### Example 104

### 1-{[(3S)-1-(Cyclopropylcarbonyl)-3-pyrrolidinyl]methyl}-N-methyl-2-[4′-(methyloxy)-4-biphenylyl]-1H-benzimidazole-6-carboxamide

### (a) Methyl 1-{[(3S)-1-(cyclopropylcarbonyl)-3-pyrrolidinyl]methyl}-2-[4′-(methyloxy)-4-biphenylyl]-1H-benzimidazole-6-carboxylate

The titled compound was prepared according to the procedure in Example 102(f), substituting 4-methoxyphenylboronic acid, to afford the desired product.

### (b) 1-{[(3S)-1-(Cyclopropylcarbonyl)-3-pyrrolidinyl]methyl}-2-[4′-(methyloxy)-4-biphenylyl]-1H-benzimidazole-6-carboxylic acid

Methyl 1-{[(3S)-1-(cyclopropylcarbonyl)-3-pyrrolidinyl]methyl}-2-[4′-(methyloxy)-4-biphenylyl]-1H-benzimidazole-6-carboxylate (418 mg) was dissolved in 8 mL THF. To this was added LiOH (35 mg in 2 mL water) and the reaction mixture stirred at RT for 15 hr. Additional LiOH was then added (70 mg) and the reaction stirred for an additional 24 hr at RT. The reaction mixture was acidified to pH ˜2 with NaHSO₄ and extracted with EtOAc (3×25 mL). The combined extracts were dried over magnesium sulfate and evaporated to afford the titled compound.

### (c) 1-{[(3S)-1-(Cyclopropylcarbonyl)-3-pyrrolidinyl]methyl}-N-methyl-2-[4′-(methyloxy)-4-biphenylyl]-1H-benzimidazole-6-carboxamide

1-{[(3S)-1-(Cyclopropylcarbonyl)-3-pyrrolidinyl]methyl}-2-[4′-(methyloxy)-4-biphenylyl]-1H-benzimidazole-6-carboxylic acid (186 mg), HATU (172 mg), methylamine (2 M in THF, 282 uL) and TEA (63 uL) were dissolved in 4 mL DMF and stirred for 1.5 hr at RT. An addition 300 uL methylamine (2 M in THF) was then added and the reaction mixture stirred for 25 hr at RT. The reaction mixture was directly purified by preparative reverse phase HPLC. The appropriate fractions were combined and lyophilized to afford 100 mg of the titled compound

### Example 105

### 1-{[(3S)-1-(Cyclopropylcarbonyl)-3-pyrrolidinyl]methyl}-2-(4′-fluoro-4-biphenylyl)-6-[(4-methyl-1-piperazinyl)carbonyl]-1H-benzimidazole

### (a) 2-(4-Bromophenyl)-1-{[(3S)-1-(cyclopropylcarbonyl)-3-pyrrolidinyl]methyl}-1H-benzimidazole-6-carboxylic acid

Methyl 2-(4-bromophenyl)-1-{[(3S)-1-(cyclopropylcarbonyl)-3-pyrrolidinyl]methyl}-1H-benzimidazole-6-carboxylate (790 mg) was dissolved in 10 mL THF. To this was added LiOH (137 mg in 2 mL water) and the reaction mixture was stirred at RT for overnight. The reaction was neutralized by adding 1 eq NaHSO4. The sample was concentrated by evaporated to remove the THF and the residual aqueous solution was extracted with EtOAc. The combined extracts were washed with brine, dried over sodium sulfate and evaporated to dryness to afford 750 mg of the titled compound, which was used without further purification.

### (b) 2-(4-Bromophenyl)-1-{[(3S)-1-(cyclopropylcarbonyl)-3-pyrrolidinyl]methyl}-6-[(4-methyl-1-piperazinyl)carbonyl]-1H-benzimidazole

2-(4-Bromophenyl)-1-{[(3S)-1-(cyclopropylcarbonyl)-3-pyrrolidinyl]methyl}-1H-benzimidazole-6-carboxylic acid (750 mg), HOBt (270 mg) and EDC (338 mg) were dissolved in 10 mL DMF and stirred for 20 min at RT. To this was added 1-methylpiperazine (160 mg) and the reaction was stirred overnight at RT. The reaction mixture was treated with additional 1-methylpiperazine, HOBt and EDC until starting material had been consumed. The solvent was removed under vacuum and the residue partitioned between EtOAc and aqueous NaHCO3. The aqueous layer was extracted twice with EtOAc and the combined EtOAc extracts were dried over sodium sulfate and evaporated to dryness. The crude product was purified by flash chromatography on silica gel using EtOH/EtOAc/1% TEA to afford 440 mg of the titled product.

### (c) 1-{[(3S)-1-(Cyclopropylcarbonyl)-3-pyrrolidinyl]methyl}-2-(4′-fluoro-4-biphenylyl)-6-[(4-methyl-1-piperazinyl)carbonyl]-1H-benzimidazole

2-(4-Bromophenyl)-1-{[(3S)-1-(cyclopropylcarbonyl)-3-pyrrolidinyl]methyl}-6-[(4-methyl-1-piperazinyl)carbonyl]-1H-benzimidazole (110 mg), 4-fluorophenylboronic acid (33.6 mg), (Ph3P)₄Pd (23.1 mg) and sodium bicarbonate (50.4 mg) were suspended in 2 mL DMF and 2 mL water in a sealed vessel and stirred at 80° C. overnight. And additional aliquot of 4-fluorophenylboronic acid (5 mg) and (Ph3P)₄Pd (2 mg) were added and the reaction stirred an additional 2 hr at 80° C. The reaction mixture was allowed to cool, diluted with water and extracted with EtOAc. The combined EtOAc extracts were washed with brine, dried over sodium sulfate and evaporated to dryness. The crude product was purified by preparative reverse phase HPLC. The appropriate fractions were combined and lyophilized to afford 105 mg of the titled compound as the trifluoroacetate salt (LCMS m/z 566.3, M+H).

### Example 106

### 1-{[(3S)-1-(Cyclopropylcarbonyl)-3-pyrrolidinyl]methyl}-2-[4-(1H-indol-6-yl)phenyl]-6-[(4-methyl-1-piperazinyl)carbonyl]-1H-benzimidazole

2-(4-Bromophenyl)-1-{[(3S)-1-(cyclopropylcarbonyl)-3-pyrrolidinyl]methyl}-6-[(4-methyl-1-piperazinyl)carbonyl]-1H-benzimidazole (110 mg), 6-indolylboronic acid (38.5 mg), (Ph3P)₄Pd (23.1 mg) and sodium bicarbonate (50.4 mg) were suspended in 2 mL DMF and 2 mL in a sealed vessel and heated at 80° C. overnight. The reaction mixture was allowed to cool, diluted with water and extracted with EtOAc. The combined EtOAc extracts were washed with brine, dried over sodium sulfate and evaporated to dryness. The crude product was purified by preparative reverse phase HPLC. The appropriate fractions were combined and lyophilized to afford 113 mg of the titled compound as a trifluoroacetate salt (LCMS m/z 587.3, M+H).

### Example 107

### 1-{[(3S)-1-(Cyclopropylcarbonyl)-3-pyrrolidinyl]methyl}-2-[4-(1H-indol-5-yl)phenyl]-6-[(4-methyl-1-piperazinyl)carbonyl]-1H-benzimidazole

2-(4-Bromophenyl)-1-{[(3S)-1-(cyclopropylcarbonyl)-3-pyrrolidinyl]methyl}-6-[(4-methyl-1-piperazinyl)carbonyl]-1H-benzimidazole (294 mg), 5-indolylboronic acid (103 mg), (Ph3P)₄Pd (61.7 mg) and sodium bicarbonate (135 mg) were suspended in 4 mL DMF and 4 mL in a sealed vessel and heated at 80° C. overnight. The reaction mixture was allowed to cool, diluted with water and extracted with EtOAc. The combined EtOAc extracts were washed with brine, dried over sodium sulfate and evaporated to dryness. The crude product was purified by preparative reverse phase HPLC. The appropriate fractions were combined, made alkaline by the addition of aqueous NaHCO3, and extracted with DCM. Removal of solvent from the DCM extracts afforded 190 mg of the titled compound (LCMS m/z 587, M+H).

### Example 108

### 1-{[(3S)-1-(Cyclopropylcarbonyl)-3-pyrrolidinyl]methyl}-7-methyl-2-[4′-(methyloxy)-4-biphenylyl]-1H-benzimidazole

### (a) 1,1-Dimethylethyl (3S)-3-{[(2-methyl-6-nitrophenyl)amino]methyl}-1-pyrrolidinecarboxylate

2-Fluoro-3-nitrotoluene (5 g) was dissolved in 30 mL dry DMSO. To this was added DIEA (5.80 g) and 1,1-dimethylethyl (3S)-3-(aminomethyl)-1-pyrrolidinecarboxylate (3.49 g). The reaction flask was flushed with nitrogen and the reaction mixture stirred at 70° C. overnight. After cooling, the reaction mixture was diluted with EtOAc and washed with brine. The EtOAc layer was dried and the solvent removed by evaporation. The crude product was purified by silica gel column chromatography using petroleum ether/EtOAc to afford the titled compound.

### (b) N-{[(3S)-1-(cyclopropylcarbonyl)-3-pyrrolidinyl]methyl}-2-methyl-6-nitroaniline

1,1-Dimethylethyl (3S)-3-{[(2-methyl-6-nitrophenyl)amino]methyl}-1-pyrrolidinecarboxylate (7 g) was dissolved in 1,4-dioxane and treated with 50 mL 4 N HCl/1,4-dioxane at RT for 3 hr. Removal of solvent gave 5.3 g of the amine hydrochloride salt, which was dissolved in 30 mL dry DCM and cooled to 0° C. TEA (3.92 g) was added and the reaction mixture was stirred for 15 min at 0° C. Cyclopropylcarbonyl chloride (2.02 g) was then added dropwise. The reaction mixture was allowed to warm to room temperature and stirred overnight. The reaction mixture was diluted with DCM, washed with brine and aqueous NH4Cl, dried over sodium sulfate and evaporated to dryness to afford 6.09 g of the titled compound, which was used without further purification.

### (c) N²-{[(3S)-1-(cyclopropylcarbonyl)-3-pyrrolidinyl]methyl}-3-methyl-1,2-benzenediamine

To a suspension of 4 g 10% Pd/C in MeOH was added N-{[(3S)-1-(cyclopropylcarbonyl)-3-pyrrolidinyl]methyl}-2-methyl-6-nitroaniline (5.7 g in MeOH) such that the total volume of the reaction was 80 mL. The reaction mixture was stirred under a hydrogen atmosphere for 5 hr, filtered and evaporated to dryness to afford 4.3 g of the titled compound, which was used without further purification.

### (d) 1-{[(3S)-1-(Cyclopropylcarbonyl)-3-pyrrolidinyl]methyl}-7-methyl-2-[4′-(methyloxy)-4-biphenylyl]-1H-benzimidazole

N2-{[(3S)-1-(cyclopropylcarbonyl)-3-pyrrolidinyl]methyl}-3-methyl-1,2-benzenediamine (150 mg) was dissolved in 2 mL DMF containing 2 drops of water. To this was added 4-(4′-methoxyphenyl)benzaldehyde (116 mg) and the reaction was stirred for 30 min. Oxone (220 mg) was then added and the reaction mixture stirred overnight at RT. The reaction mixture was filtered and directly purified by preparative reverse phase HPLC to afford 80 mg of the titled compound

### Example 109

### 1-{[(3S)-1-(Cyclopropylcarbonyl)-3-pyrrolidinyl]methyl}-2-[4-(1H-indol-5-yl)phenyl]-7-methyl-1H-benzimidazole

The titled compound was prepared according to the procedure in Example 108(d), substituting 4-(1H-indol-5-yl)benzaldehyde, to afford 50 mg of the desired product

### Example 110

### 1-{[(3S)-1-(Cyclopropylcarbonyl)-3-pyrrolidinyl]methyl}-2-[4-(1H-indol-6-yl)phenyl]-7-methyl-1H-benzimidazole

The titled compound was prepared according to the procedure in Example 108(d), substituting 4-(1H-indol-6-yl)benzaldehyde, to afford 40 mg of the desired product

### Example 111

### 5-[4-(1-{[(3S)-1-(Cyclopropylcarbonyl)-3-pyrrolidinyl]methyl}-7-methyl-1H-benzimidazol-2-yl)phenyl]-1H-indazole

The titled compound was prepared according to the procedure in Example 108(d), substituting 5-(4-formylphenyl)-1H-indazole, to afford 30 mg of the desired product

### Example 112

### 2-(4-Biphenylyl)-1-{[(3S)-1-(cyclopropylcarbonyl)-3-pyrrolidinyl]methyl}-7-methyl-1H-benzimidazole

The titled compound was prepared according to the procedure in Example 108(d), substituting 4-biphenylcarbaldehyde, to afford 90 mg of the desired product

### Example 113

### 4′-(1-{[(3S)-1-(Cyclopropylcarbonyl)-3-pyrrolidinyl]methyl}-7-methyl-1H-benzimidazol-2-yl)-3-biphenylol

The titled compound was prepared according to the procedure in Example 108(d), substituting 4-(3′-hydroxyphenyl)benzaldeyde, to afford 40 mg of the desired product

### Example 114

### 1-{[(3S)-1-(Cyclopropylcarbonyl)-3-pyrrolidinyl]methyl}-2-(4′-fluoro-4-biphenylyl)-7-methyl-1H-benzimidazole

The titled compound was prepared according to the procedure in Example 108(d), substituting 4-(4′-fluorophenyl)benzaldehyde, to afford 100 mg of the desired product

### Example 115

### 6-[4-(1-{[(3S)-1-(Cyclopropylcarbonyl)-3-pyrrolidinyl]methyl}-7-methyl-1H-benzimidazol-2-yl)phenyl]-1H-indazole

N2-{[(3S)-1-(cyclopropylcarbonyl)-3-pyrrolidinyl]methyl}-3-methyl-1,2-benzenediamine (150 mg) was dissolved in 3 mL DMF containing 2 drops of water. To this was added 6-(4-formylphenyl)-1H-indazole (61 mg) and the reaction was stirred for 30 min. Oxone (220 mg) was then added and the reaction mixture stirred overnight at RT. The reaction mixture was filtered and directly purified by preparative reverse phase HPLC to afford 50 mg of the titled compound (LCMS m/z 476.2, M+H).

### Example 116

### 2-[4-(1-Benzofuran-5-yl)phenyl]-1-{[(3S)-1-(cyclopropylcarbonyl)-3-pyrrolidinyl]methyl}-7-methyl-1H-benzimidazole

N2-{[(3S)-1-(cyclopropylcarbonyl)-3-pyrrolidinyl]methyl}-3-methyl-1,2-benzenediamine (90 mg) was dissolved in 2 mL DMF. To this was added (1-benzofuran-5-yl)benzaldehyde (80 mg) and oxone (144 mg) and the reaction mixture was stirred at RT overnight. The reaction mixture was directly purified by preparative reverse phase HPLC to afford 56 mg of the titled compound (LCMS m/z 476.3, M+H).

### Example 117

### 1-{[(3S)-1-(Cyclopropylcarbonyl)-3-pyrrolidinyl]methyl}-2-(4′-fluoro-4-biphenylyl)-7-(trifluoromethyl)-1H-benzimidazole

### (a) 1,1-Dimethylethyl (3S)-3-({[2-nitro-6-(trifluoromethyl)phenyl]amino}methyl)-1-pyrrolidinecarboxylate

2-Fluoro-1-nitro-3-(trifluoromethyl)benzene (5 g), 1,1-dimethylethyl (3S)-3-(aminomethyl)-1-pyrrolidinecarboxylate (5.3 g) and DIEA (4.3 g) were combined in 50 mL DMSO and stirred at 70° C. overnight. The reaction mixture was allowed to cool, diluted with water and extracted several times with EtOAc. The combined EtOAc extracts were washed with saturated brine, evaporated to dryness and purified by silica gel column chromatography using petroleum ether/EtOAc to afford the titled compound.

### (b) N-{[(3S)-1-(Cyclopropylcarbonyl)-3-pyrrolidinyl]methyl}-2-nitro-6-(trifluoromethyl)aniline

1,1-Dimethylethyl (3S)-3-({[2-nitro-6-(trifluoromethyl)phenyl]amino}methyl)-1-pyrrolidinecarboxylate (10 g) was dissolved in MeOH. 5 N HCl/MeOH (25 mL) was added and the reaction mixture stirred at RT for 2 hr. Removal of the solvent under reduced pressure afforded 7 g of the amine hydrochloride salt, which was dissolved in 50 mL DCM. DIEA (3.6 g) was added dropwise at RT. The reaction mixture was stirred for 30 min at RT and then cyclopropylcarbonyl chloride (2.24 g) was added dropwise and the reaction mixture stirred at RT overnight. The solvent was removed under reduced pressure and the crude product was purified by silica gel column chromatography to afford 5.5 g of the titled compound.

### (c) N²-{[(3S)-1-(Cyclopropylcarbonyl)-3-pyrrolidinyl]methyl}-3-(trifluoromethyl)-1,2-benzenediamine

N-{[(3S)-1-(Cyclopropylcarbonyl)-3-pyrrolidinyl]methyl}-2-nitro-6-(trifluoromethyl)aniline (1 g) was dissolved in 20 mL MeOH. To this was added 500 mg 10% Pd/C and the reaction mixture was stirred at RT overnight under a hydrogen atmosphere (balloon). The reaction mixture was filtered and the solvent removed under reduced pressure to afford the titled compound, which was used without further purification.

### (d) 1-{[(3S)-1-(Cyclopropylcarbonyl)-3-pyrrolidinyl]methyl}-2-(4′-fluoro-4-biphenylyl)-7-(trifluoromethyl)-1H-benzimidazole

N2-{[(3S)-1-(Cyclopropylcarbonyl)-3-pyrrolidinyl]methyl}-3-(trifluoromethyl)-1,2-benzenediamine (120 mg) was dissolved in 3 mL DMF. To this was added 4-(4′-fluorophenyl)benzaldehyde (58.7 g) and oxone (146.13 g) and the reaction mixture was stirred at RT overnight. The reaction mixture was diluted with 10 mL water and extracted with EtOAc (2×30 mL). The combined EtOAc extracts were washed with brine, dried over sodium sulfate and evaporated to dryness. The crude product was purified by preparative reverse phase HPLC to afford 53 mg of the titled compound (LCMS m/z 508.3, M+H).

### Example 118

### 1-{[(3S)-1-(Ccyclopropylcarbonyl)-3-pyrrolidinyl]methyl}-2-[4′-(methyloxy)-4-biphenylyl]-7-(trifluoromethyl)-1H-benzimidazole

The titled compound was prepared according to the procedure in Example 117(d), substituting 4-(4′-methoxyphenyl)benzaldehyde, to afford 52 mg of the desired product (LCMS m/z 520.3, M+H).

### Example 119

### 2-[4-(1-Benzofuran-5-yl)phenyl]-1-{[(3S)-1-(cyclopropylcarbonyl)-3-pyrrolidinyl]methyl}-7-(trifluoromethyl)-1H-benzimidazole

The titled compound was prepared according to the procedure in Example 117(d), starting with 150 mg of N2-{[(3S)-1-(cyclopropylcarbonyl)-3-pyrrolidinyl]methyl}-3-(trifluoromethyl)-1,2-benzenediamine and substituting (1-benzofuran-5-yl)benzaldehyde, to afford 118 mg of the desired product (LCMS m/z 530.3, M+H).

### Example 120

### 1-{[(3S)-1-(Cyclopropylcarbonyl)-3-pyrrolidinyl]methyl}-2-[4-(1H-indol-5-yl)phenyl]-7-(trifluoromethyl)-1H-benzimidazole

The titled compound was prepared according to the procedure in Example 117(d), starting with 150 mg of N2-{[(3S)-1-(cyclopropylcarbonyl)-3-pyrrolidinyl]methyl}-3-(trifluoromethyl)-1,2-benzenediamine and substituting 4-(1H-indol-5-yl)benzaldehyde, to afford 30 mg of the desired compound (LCMS m/z 529.3, M+H).

### Example 121

### 5-{4-[1-{[(3S)-1-(Cyclopropylcarbonyl)-3-pyrrolidinyl]methyl}-7-(trifluoromethyl)-1H-benzimidazol-2-yl]phenyl}-1H-indazole

The titled compound was prepared according to the procedure in Example 117(d), starting with 150 mg of N2-{[(3S)-1-(cyclopropylcarbonyl)-3-pyrrolidinyl]methyl}-3-(trifluoromethyl)-1,2-benzenediamine and substituting 5-(4-formylphenyl)-1H-indazole, to afford 96 mg of the desired product (LCMS m/z 530.3, M+H).

### Example 122

### 6-{4-[1-{[(3S)-1-(Cyclopropylcarbonyl)-3-pyrrolidinyl]methyl}-7-(trifluoromethyl)-1H-benzimidazol-2-yl]phenyl}-1H-indazole

The titled compound was prepared according to the procedure in Example 117(d), starting with 150 mg of N2-{[(3S)-1-(cyclopropylcarbonyl)-3-pyrrolidinyl]methyl}-3-(trifluoromethyl)-1,2-benzenediamine and substituting 6-(4-formylphenyl)-1H-indazole, to afford 52 mg of the desired product (LCMS m/z 530.3, M+H).

### Example 123

### 1-{[(3S)-1-(Cyclopropylcarbonyl)-3-pyrrolidinyl]methyl}-2-[4-(1H-indol-6-yl)phenyl]-7-(trifluoromethyl)-1H-benzimidazole

The titled compound was prepared according to the procedure in Example 117(d), starting with 150 mg of N2-{[(3S)-1-(cyclopropylcarbonyl)-3-pyrrolidinyl]methyl}-3-(trifluoromethyl)-1,2-benzenediamine and substituting 4-(1H-indol-6-yl)benzaldehyde, to afford 32 mg of the desired product (LCMS m/z 529.3, M+H).

### Example 124

### 2-(4-Biphenylyl)-7-bromo-1-{[(3S)-1-(cyclopropylcarbonyl)-3-pyrrolidinyl]methyl}-1H-benzimidazole

### (a) 1,1-Dimethylethyl (3S)-3-{[(2-bromo-6-nitrophenyl)amino]methyl}-1-pyrrolidinecarboxylate

1-Bromo-2-fluoro-3-nitrobenzene (5 g), 1,1-dimethylethyl (3S)-3-(aminomethyl)-1-pyrrolidinecarboxylate (4.62 g) and DIEA (7.4 g) were dissolved in 50 mL DMSO and stirred at 70° C. overnight. The reaction mixture was allowed to cool, and then was diluted with water and extracted several times with EtOAc. The combined EtOAc extracts were washed with water and brine, dried over sodium sulfate and then evaporated under reduced pressure. The crude product was purified by silica gel column chromatography using petroleum ether/EtOAc to afford 7.1 g of the titled compound.

### (b) (2-Bromo-6-nitrophenyl) {[(3S)-1-(cyclopropylcarbonyl)-3-pyrrolidinyl]methyl}amine

1,1-Dimethylethyl (3S)-3-{[(2-bromo-6-nitrophenyl)amino]methyl}-1-pyrrolidinecarboxylate (7.1 g) was dissolved in MeOH. 5N HCl/MeOH (250 mL) was slowly added and the reaction mixture was stirred at RT for 2 hr. Removal of the solvent under reduced pressure gave the amine hydrochloride salt, which was dissolved in 100 mL DCM. TEA (5.4 g) was added and the reaction mixture stirred at RT for 10 min. Cyclopropylcarbonyl chloride (2.05 g) was then added and the reaction mixture stirred at RT overnight. The reaction mixture was washed with water and brine, dried over sodium sulfate and evaporated under reduced pressure to afford 6.57 g of the titled compound, which was used without further purification.

### (c) 2-Amino-6-bromophenyl){[(3S)-1-(cyclopropylcarbonyl)-3-pyrrolidinyl]methyl}amine

(2-Bromo-6-nitrophenyl) {[(3S)-1-(cyclopropylcarbonyl)-3-pyrrolidinyl]methyl}amine (6.57 g) and SnCl2.2H2O (14.1 g) were dissolved in 200 mL EtOAc and heated at reflux overnight. The reaction mixture was allowed to cool and saturated aqueous NaOH (14 eq) was added and stirred at RT for 40 min. The organic layer was separated, dried over sodium sulfate and evaporated under reduced pressure to afford the titled compound, which was used without further purification.

### (d) 2-(4-Biphenylyl)-7-bromo-1-{[(3S)-1-(cyclopropylcarbonyl)-3-pyrrolidinyl]methyl}-1H-benzimidazole

2-Amino-6-bromophenyl) {[(3S)-1-(cyclopropylcarbonyl)-3-pyrrolidinyl]methyl}amine (120 mg) and, 4-biphenylcarbaldehyde (65 mg) were dissolved in 4 mL DMF and stirred for a few minutes. Oxone (141 mg) was then added and the reaction mixture stirred at RT overnight. The reaction mixture was filtered and directly purified by preparative reverse phase HPLC to afford 83 mg of the titled compound. (LCMS m/z 500.0, M+H).

### Example 125

### 4′-(7-Bromo-1-{[(3S)-1-(cyclopropylcarbonyl)-3-pyrrolidinyl]methyl}-1H-benzimidazol-2-yl)-3-biphenylol

The titled compound was prepared according to the procedure in Example 124(d), substituting 4-(3′-hydroxyphenyl)benzaldehyde, to afford 50 mg of the desired product. (LCMS m/z 516.1, M+H).

### Example 126

### 7-Bromo-1-{[(3S)-1-(cyclopropylcarbonyl)-3-pyrrolidinyl]methyl}-2-(4′-fluoro-4-biphenylyl)-1H-benzimidazole

The titled compound was prepared according to the procedure in Example 124(d), substituting 4-(4′-fluorophenyl)benzaldehyde, to afford 64 mg of the desired product (LCMS m/z 518.1, M+H)).

### Example 127

### 7-Bromo-1-{[(3S)-1-(cyclopropylcarbonyl)-3-pyrrolidinyl]methyl}-2-[4′-(methyloxy)-4-biphenylyl]-1H-benzimidazole

The titled compound was prepared according to the procedure in Example 223(d), substituting 4-(4′-methoxyphenyl)benzaldehyde, to afford 87 mg of the desired product. (LCMS m/z 530.1, M+H).

### Example 128

### 7-Bromo-1-{[(3S)-1-(cyclopropylcarbonyl)-3-pyrrolidinyl]methyl}-2-[4-(1H-indol-5-yl)phenyl]-1H-benzimidazole

The titled compound was prepared according to the procedure in Example 124(d), substituting 4-(1H-indol-5-yl)benzaldehyde, to afford 20 mg of the desired product. (LCMS m/z 539.1, M+H).

### Example 129

### 5-[4-(7-Bromo-1-{[(3S)-1-(cyclopropylcarbonyl)-3-pyrrolidinyl]methyl}-1H -benzimidazol-2-yl)phenyl]-1H-indazole

The titled compound was prepared according to the procedure in Example 124(d), substituting 5-(4-formylphenyl)-1H-indazole, to afford 67 mg of the desired product. (LCMS m/z 540.1, M+H).

### Example 130

### 7-Bromo-1-{[(3S)-1-(cyclopropylcarbonyl)-3-pyrrolidinyl]methyl}-2-[4-(1H-indol-6-yl)phenyl]-1H-benzimidazole

The titled compound was prepared according to the procedure in Example 124(d), substituting 4-(1H-indol-6-yl)benzaldehyde, to afford 16 mg of the desired product. (LCMS m/z 539.1, M+H).

### Example 131

### 6-[4-(7-Bromo-1-{[(3S)-1-(cyclopropylcarbonyl)-3-pyrrolidinyl]methyl}-1H-benzimidazol-2-yl)phenyl]-1H-indazole

The titled compound was prepared according to the procedure in Example 124(d), substituting 6-(4-formylphenyl)-1H-indazole, to afford 38 mg of the desired product. (LCMS m/z 540.2, M+H).

### Example 132

### 2-[4-(1-Benzofuran-5-yl)phenyl]-7-bromo-1-{[(3S)-1-(cyclopropylcarbonyl)-3-pyrrolidinyl]methyl}-1H-benzimidazole

The titled compound was prepared according to the procedure in Example 124(d), substituting (1-benzofuran-5-yl)benzaldehyde, to afford 36.7 mg of the desired compound (LCMS m/z 540.2, M+H).

### Example 133

### 1-{[(3S)-1-(cyclopropylcarbonyl)-3-pyrrolidinyl]methyl}-2-(3′-hydroxy-4-biphenylyl)-N-methyl-1H-benzimidazole-7-carboxamide

### (a) 2-Fluoro-3-nitrobenzoyl chloride

To a mixture of 2-fluoro-3-nitrobenzoic acid (1 g) and thionyl chloride (20 mL) was added 2 drops of DMF. The reaction mixture was heated at reflux overnight, allowed to cool and then evaporated under reduced pressure to afford the titled compounds, which was used without further purification.

### (b) 2-Fluoro-N-methyl-3-nitrobenzamide

Methylamine hydrochloride (330 mg) was suspended in 5 mL DCM. To this was added TEA (1.23 g) and the mixture was stirred at RT for 30 min. The reaction mixture was then cooled to −78° C. and DMAP (15 mg) was added. 2-Fluoro-3-nitrobenzoyl chloride (1.0 g in 5 mL DCM) was added in one portion and the reaction mixture was stirred for 30 min. The reaction mixture was diluted with DCM and extracted with aqueous NH4Cl, aqueous NaHCO3, water and brine. The organic layer was dried over sodium sulfate and evaporated under reduced pressure to afford the titled compound, which was used without further purification.

### (c) 1,1-Dimethylethyl (3S)-3-[([2-[(methylamino)carbonyl]-6-riltrophenyl]amino)methyl]-1-pyrrolidinecarboxylate

2-Fluoro-N-methyl-3-nitrobenzamide (4.0 g) was dissolved in 40 mL DMSO. To this was added 1,1-dimethylethyl (3S)-3-(aminomethyl)-1-pyrrolidinecarboxylate (4.86 g) and DIEA (3.91 g) and the reaction mixture was stirred at 90° C. overnight. The reaction mixture was allowed to cool and was then diluted with water and extracted with EtOAc. The combined extracts were washed with water and brine, dried over sodium sulfate, and evaporated under reduced pressure. The crude product was purified by silica gel column chromatography using petroleum ether/EtOAc to afford 7.42 g of the titled compound.

### (d) 2-({[(3S)-1-(Cyclopropylcarbonyl)-3-pyrrolidinyl]methyl}amino)-N-methyl-3-nitrobenzamide

To 1,1-dimethylethyl (3S)-3-[({2-[(methylamino)carbonyl]-6-nitrophenyl}amino)methyl]-1-pyrrolidinecarboxylate (7.42 g) was added 70 mL 5 N HCl/MeOH and the reaction mixture was stirred at RT for 4 hr. The reaction mixture was evaporated under reduced pressure to give the amine hydrochloride salt, which was dissolved in 70 mL dry DCM. DIEA (8.82 g) was added and the reaction mixture was stirred at RT for 30 min. Cyclopropylcarbonyl chloride (3.57 g) was then added dropwise and the reaction mixture stirred at RT overnight. Saturated aqueous NaHCO3 was then added dropwise and the reaction mixture was extracted with EtOAc. The combined EtOAc extracts were washed with water and brine, dried over sodium sulfate, and evaporated under reduced pressure to afford 7.2 g of the titled compound, which was used without further purification.

### (e) 3-Amino-2-({[(3S)-1-(cyclopropylcarbonyl)-3-pyrrolidinyl]methyl}amino)-N-methylbenzamide

2-({[(3S)-1-(Cyclopropylcarbonyl)-3-pyrrolidinyl]methyl}amino)-N-methyl-3-nitrobenzamide (7.1 g) was dissolved in 70 mL MeOH. To this was added 4 g 10% Pd/C (Degussa) under argon. The reaction mixture was stirred at RT overnight under a hydrogen atmosphere. The reaction mixture was filtered and evaporated under reduced pressure to afford 4.93 g of the titled compound, which was used without further purification.

### (f) 1-{[(3S)-1-(cyclopropylcarbonyl)-3-pyrrolidinyl]methyl}-2-(3′-hydroxy-4-biphenylyl)-N-methyl-1H-benzimidazole-7-carboxamide

3-Amino-2-({[(3S)-1-(cyclopropylcarbonyl)-3-pyrrolidinyl]methyl}amino)-N-methylbenzamide (120 mg) was dissolved in 4 mL DMF. To this was added 4-(3′-hydroxyphenyl)benzaldehyde (75.2 mg) and oxone (151.7 mg) and the reaction mixture was stirred overnight at RT. The reaction mixture was filtered and purified directly by preparative reverse phase HPLC to afford 81 mg of the titled compound (LCMS m/z 495.4, M+H).

### Example 134

### 1-{[(3S)-1-(Cyclopropylcarbonyl)-3-pyrrolidinyl]methyl}-2-(4′-fluoro-4-biphenylyl)-N-methyl-1H-benzimidazole-7-carboxamide

The titled compound was prepared according to the procedure in Example 133(f), substituting 4-(4′-fluorophenyl)benzaldehyde, to afford 105 mg of the desired product (LCMS m/z 497.4, M+H).

### Example 135

### 1-{[(3S)-1-(Cyclopropylcarbonyl)-3-pyrrolidinyl]methyl}-2-[4-(1H-indol-5-yl)phenyl]-N-methyl-1H-benzimidazole-7-carboxamide

The titled compound was prepared according to the procedure in Example 133(f), substituting 4-(1H-indol-5-yl)benzaldehyde, to afford 57.9 mg of the desired product (LCMS m/z 518.3, M+H).

### Example 136

### 1-{[(3S)-1-(Cyclopropylcarbonyl)-3-pyrrolidinyl]methyl}-2-[4-(1H-indol-6-yl)phenyl]-N-methyl-1H-benzimidazole-7-carboxamide

The titled compound was prepared according to the procedure in Example 133(f), substituting 4-(1H-indol-6-yl)benzaldehyde, to afford 31 mg of the desired product (LCMS m/z 518.3, M+H).

### Example 137

### 1-{[(3S)-1-(Cyclopropylcarbonyl)-3-pyrrolidinyl]methyl}-2-[4-(1H-indazol-5-yl)phenyl]-N-methyl-1H-benzimidazole-7-carboxamide

The titled compound was prepared according to the procedure in Example 133(f), substituting 5-(4-formylphenyl)-1H-indazole, to afford 31 mg of the desired product (LCMS m/z 519.3, M+H).

### Example 138

### 1-{[(3S)-1-(Cyclopropylcarbonyl)-3-pyrrolidinyl]methyl}-2-[4-(1H-indazol-6-yl)phenyl]-N-methyl-1H-benzimidazole-7-carboxamide

The titled compound was prepared according to the procedure in Example 133(f), substituting 6-(4-formylphenyl)-1H-indazole, to afford 66 mg of the desired product (LCMS m/z 519.3, M+H).

### Example 139

### 2-(4-Diphenylyl)-1-{[(3S)-1-(cyclopropylcarbonyl)-3-pyrrolidinyl]methyl}-N-methyl-1H-benzimidazole-7-carboxamide

The titled compound was prepared according to the procedure in Example 133(f), substituting 4-biphenylcarbaldehyde, to afford 100 mg of the desired product (LCMS m/z 479.1, M+H).

### Example 140

### 1-{[(3S)-1-(Cyclopropylcarbonyl)-3-pyrrolidinyl]methyl}-N-methyl-2-[4′-(methyloxy)-4-biphenylyl]-1H-benzimidazole-7-carboxamide

The titled compound was prepared according to the procedure in Example 133(f), substituting 4-(4-methoxyphenyl)benzaldehyde, to afford 37 mg of the desired product (LCMS m/z 509.4, M+H).

### Example 141

### 2-[4-(1-Benzofuran-5-yl)phenyl]-1-{[(3S)-1-(cyclopropylcarbonyl)-3-pyrrolidinyl]methyl}-N-methyl-1H-benzimidazole-7-carboxamide

3-Amino-2-({[(3S)-1-(cyclopropylcarbonyl)-3-pyrrolidinyl]methyl}amino)-N-methylbenzamide (150 mg) was dissolved in 2 mL DMF. To this was added (1-benzofuran-5-yl)benzaldehyde (105.2 g) and oxone (189.1 g) and the reaction mixture was stirred at RT overnight. The reaction mixture was diluted with 10 mL water and extracted with EtOAc (2×30 mL). The combined EtOAc extracts were washed with brine, dried over sodium sulfate and evaporated to dryness. The crude product was purified by preparative reverse phase HPLC to afford 112 mg of the titled (LCMS m/z 519.4, M+H).

### Example 142

### 2-(4-Biphenylyl)-1-({(3RS)-1-[(dimethylamino)carbonyl]-3-pyrrolidinyl}methyl)-N-methyl-1H-benzimidazole-6-carboxamide

### (a) 3-{[(3RS)-1-{[(1,1-Dimethylethyl)oxy]carbonyl}-3-pyrrolidinyl)methyl]amino}-4-nitrobenzoic acid

3-Fluoro-4-nitrobenzoic acid (4.77 g) and 1,1-dimethylethyl (3RS)-3-(aminomethyl)-1-pyrrolidinecarboxylate (5.0 g) were dissolved in 25 mL DMSO and stirred at 98° C. under nitrogen for 17 hr. LCMS indicated a mixture of the desired product and the des-Boc product. The reaction mixture was allowed to cool and 125 mL water and 2.2 g NaOH were added. The reaction mixture was cooled to 0° C. and di-t-butyl dicarbonate (5.46 g) was added. The reaction mixture was allowed to warm to RT and stirred for 20 hr. The reaction mixture was neutralized by the addition of 1 N HCl, saturated with NaCl, and extracted with DCM/diethyl ether. The combined organic extracts were dried over sodium sulfate and evaporated under reduced pressure to afford the titled compound, which was used without further purification.

### (b) 2-(4-Biphenylyl)-1-[((3RS)-1-{[(1,1-dimethylethyl)oxy]carbonyl}-3-pyrrolidinyl)methyl]-1H-benzimidazole-6-carboxylic acid

3-{[((3RS)-1-{[(1,1-Dimethylethyl)oxy]carbonyl}-3-pyrrolidinyl)methyl]amino}-4-nitrobenzoic acid (9.1 g) and 4-biphenylcarbaldehyde (4.8 g) were suspended in 25 mL DMSO and 100 mL EtOH. To this was added sodium hydrosulfite (15.4 g) and the reaction mixture was stirred at 80° C. for 23 hr. The reaction mixture was allowed to cool and the EtOH was removed under reduced pressure. A 5 N solution of NH4OH (50 mL) was added dropwise followed by 125 mL water. The reaction mixture was cooled to 0° C. and stirred until a precipitate formed. The reaction mixture was filtered and the precipitate washed with cold water. The combined filtrates were acidified by the addition of 20 mL conc. HCl and extracted with THF/diethyl ether. The aqueous layer was again brought to pH 7 by the addition of 15% aqueous NaOH and extracted again with THF/diethyl ether. The combined organic extracts were dried over sodium sulfate, evaporated under reduced pressure, redissolved in EtOH and evaporated again, taken up into toluene and evaporated (2×) to afford 8.7 g of the titled compound, which was used without further purification.

### (c) 1,1-Dimethylethyl (3RS)-3-({2-(4-biphenylyl)-6-[(methylamino)carbonyl]-1H-benzimidazol-1-yl}methyl)-1-pyrrolidinecarboxylate

2-(4-Biphenylyl)-1-[((3RS)-1-{[(1,1-dimethylethyl)oxy]carbonyl}-3-pyrrolidinyl)methyl]-1H-benzimidazole-6-carboxylic acid (8.7 g) was dissolved in 44 mL THF. To this was added diisopropylcarbodiimide (4.1 mL) and 4-N,N-dimethylaminopyridine (2.1 g) and the reaction mixture was stirred for 45 min at RT. Methylamine (2 M in THF, 13 mL) was then added and the reaction mixture stirred at RT for 22 hr. Addition of another 4.1 mL diisopropylcarbodiimide and 13 mL 2 M methylamine in THF did not push the reaction any further toward completion. The reaction mixture was concentrated under reduced pressure and the residue partitioned between THF/diethyl ether and water containing 20 mmol NaOH. The organic layer was separated, washed with dilute aqueous HCl, dilute aqueous NaOH, water, and brine, was dried over sodium sulfate and evaporated under reduced pressure. The crude product was purified by flash chromatography on silica gel using MeOH/DCM to afford 1.8 g of the titled compound.

### (d) 2-(4-Biphenylyl)-N-methyl-1-[(3RS)-3-pyrrolidinylmethyl]-1H-benzimidazole-6-carboxamide

1,1-Dimethylethyl (3RS)-3-({2-(4-biphenylyl)-6-[(methylamino)carbonyl]-1H-benzimidazol-1-yl}methyl)-1-pyrrolidinecarboxylate (0.5 g) was dissolved in 5 mL DCM and 5 mL TFA and stirred for 2 hr at RT. The solvent was removed under reduced pressure and the residue partitioned between THF/diethyl ether and 0.5 M NaOH. The organic layer was separated and washed with water and brine, dried over sodium sulfated and evaporated under reduced pressure to afford 0.36 g of the titled compound, which was used without further purification.

### (e) 2-(4-Biphenylyl)-1-({(3RS)-1-[(dimethylamino)carbonyl]-3-pyrrolidinyl}methyl)-N-methyl-1H-benzimidazole-6-carboxamide

2-(4-Biphenylyl)-N-methyl-1-[(3RS)-3-pyrrolidinylmethyl]-1H-benzimidazole-6-carboxamide (32 mg), N,N-dimethylcarbamoyl chloride (12 mg), TEA (12 uL) and NMP (230 uL) were combined and stirred at RT for 3 days. The reaction mixture was then treated with tris-amine resin (3.45 mmol/g, 20 mg) and filtered. The resin was washed with NMP (2×200 uL) and MeOH (2×200 uL) and the filtrates combined. The MeOH was removed under reduced pressure and the total volume was made up to 800 uL with NMP and then purified by preparative reverse phase HPLC to afford the titled compound (LCMS m/z 482.2, M+H).

### Example 143

### 2-(4-Biphenylyl)-N-methyl-1-({(3RS)-1-[(3-methyl-5-isoxazolyl)carbonyl]-3-pyrrolidinyl}methyl)-1H-benzimidazole-6-carboxamide

2-(4-Biphenylyl)-N-methyl-1-[(3RS)-3-pyrrolidinylmethyl]-1H-benzimidazole-6-carboxamide (41 mg), DCC resin (Polymer Laboratories, 1.55 mmol/g, 84 mg) and HOBt resin (Polymer Laboratories, 1.31 mmol/g, 98 mg) were suspended in 500 uL DCM and agitated for 10 min. 3-Methyl-5-isoxazoleacetic acid (17 mg) and 44 uL DIEA in 500 uL DCM were then added and the reaction mixture agitated for 20 hr at RT. The reaction mixture was filtered and the resin washed with DCM. The combined filtrates were evaporated and the residue partitioned between EtOAc/DCM and aqueous NaHCO3. The organic layer was separated, washed with brine, dried over sodium sulfate and evaporated to dryness. The crude product was purified by flash chromatography on silica gel using MeOH/DCM to afford 2.8 mg of the titled compound (LCMS m/z 534.4, M+H).

### Example 144

### 1-{[(3S)-1-(Cyclopropylcarbonyl)-3-pyrrolidinyl]methyl}-2-[4-(1H-pyrrolo[3,2-b]pyridin-6-yl)phenyl]-5-(trifluoromethyl)-1H-benzimidazole

2-(4-Bromophenyl)-1-{[(3S)-1-(cyclopropylcarbonyl)-3-pyrrolidinyl]methyl}-5-(trifluoromethyl)-1H-benzimidazole (80 mg, 0.162 mmol) was dissolved in 1,4-dioxane (1.5 mL) in 5-mL microwave vial. Potassium acetate (43.5 mg, 0.443 mmol), 4,4,4′,4′,5,5,5′,5′-octamethyl-2,2′-bi-1,3,2-dioxaborolane (41.3 mg, 0.162 mmol), and PdCl₂(dppf)-CH₂Cl₂ adduct (6.03 mg, 7.39 umol) were then added with stirring. The vial was purged with nitrogen, sealed, and heated to 100° C. for 2 hours. The reaction mixture was allowed to cool to room temperature. 6-Bromo-1H-pyrrolo[3,2-b]pyridine (29.1 mg, 0.148 mmol), PdCl₂(dppf)-CH₂Cl₂ adduct (6.03 mg, 7.39 umol) and 2 M aqueous potassium carbonate (0.222 mL, 0.443 mmol) were then added. The vial was again purged with nitrogen, sealed and heated at 100° C. for 2 hours. The reaction mixture was allowed to cool to room temperature, diluted with water (50 mL), acidified to pH 7 with 1 N HCl, and extracted with DCM (3×50 mL). The combined DCM extracts were dried over sodium sulfate and evaporated to dryness. The resulting crude product was dissolved in DMSO and purified by preparative reverse phase hplc. The appropriate fractions were combined and the pH adjusted to 7 with aqueous sodium bicarbonate (saturated) and extracted with DCM (3×25 mL). The combined DCM extracts were dried over sodium sulfate and evaporated to dryness to afford 47 mg of the titled compound as an off-white solid. (LCMS m/z 530.0, M+H).

### Example 145

### 1-{[(3S)-1-(Cyclopropylcarbonyl)-3-pyrrolidinyl]methyl}-2-(4-imidazo[1,2-a]pyridin-7-ylphenyl)-5-(trffluoromethyl)-1H-benzimidazole

2-(4-Bromophenyl)-1-{[(3S)-1-(cyclopropylcarbonyl)-3-pyrrolidinyl]methyl}-5-(trifluoromethyl)-1H-benzimidazole (82 mg, 0.167 mmol) was dissolved in 1,4-dioxane (1.5 mL) in 5-mL microwave vial. Potassium acetate (44.8 mg, 0.457 mmol), 4,4,4′,4′,5,5,5′,5′-octamethyl-2,2′-bi-1,3,2-dioxaborolane (42.5 mg, 0.167 mmol), and

PdCl₂(dppf)-CH₂Cl₂ adduct (6.22 mg, 7.61 umol) were then added with stirring. The vial was purged with nitrogen, sealed, and heated to 100° C. for 2 hours. The reaction mixture was allowed to cool to room temperature. 7-Bromoimidazo[1,2-a]pyridine (30 mg, 0.152 mmol), PdCl₂(dppf)-CH₂Cl₂ adduct (6.22 mg, 7.61 umol) and 2 M aqueous potassium carbonate (0.228 mL, 0.457 mmol) were then added. The vial was again purged with nitrogen, sealed and heated at 100° C. for 2 hours. The reaction mixture was allowed to cool to room temperature, diluted with water (50 mL), acidified to pH 7 with 1 N HCl, and extracted with DCM (3×50 mL). The combined DCM extracts were dried over sodium sulfate and evaporated to dryness. The resulting crude product was dissolved in DMSO and purified by preparative reverse phase hplc. The appropriate fractions were combined and the pH adjusted to 7 with aqueous sodium bicarbonate (saturated) and extracted with DCM (3×25 mL). The combined DCM extracts were dried over sodium sulfate and evaporated to dryness to afford 22 mg of the titled compound. (LCMS m/z 529.9, M+H).

### Example 146

### 5-{4-[1-{[(3S)-1-(Cyclopropylcarbonyl)-3-pyrrolidinyl]methyl}-5-(trifluoromethyl)-1H-benzimidazol-2-yl]phenyl}-1H-pyrazolo[3,4-b]pyridine

The titled compound was prepared according to the procedure in Example 144, substituting 5-bromo-1H-pyrazolo[3,4-b]pyridine (29.3 mg, 0.148 mmol). The crude product was purified by silica gel column chromatography using a 0-8% MeOH/DCM gradient. The appropriate fractions were combined, evaporated to dryness, dissolved in DCM (1 mL) and stirred with SiliCycle Si-Thiol scavenger resin (20 mg) for 1 hr. The mixture was filtered and the filtrate evaporated to dryness to afford 17 mg of the titled compound. (LCMS m/z 530.9, M+H).

### Example 147

### 5-{4-[1-{[(3S)-1-(Cyclopropylcarbonyl)-3-pyrrolidinyl]methyl}-5-(trifluoromethyl)-1H-benzimidazol-2-yl]phenyl}-1,3-benzoxazole

The titled compound was prepared according to the procedure in Example 144, substituting 5-bromo-1,3-benzoxazole (29.3 mg, 0.148 mmol). The crude product was purified by silica gel column chromatography using a 0-5% MeOH/DCM gradient, and then further purified by preparative reverse phase hplc. The appropriate fractions were combined and evaporated to dryness to afford 30 mg of the titled compound. (LCMS m/z 530.8, M+H).

### Example 148

### 6-{4-[1-{[(3S)-1-(Cyclopropylcarbonyl)-3-pyrrolidinyl]methyl}-5-(trifluoromethyl)-1H-benzimidazol-2-yl]phenyl}-1,3-benzothiazole

2-(4-Bromophenyl)-1-{[(3S)-1-(cyclopropylcarbonyl)-3-pyrrolidinyl]methyl}-5-(trifluoromethyl)-1H-benzimidazole (100 mg, 0.203 mmol) was dissolved in 1,4-dioxane (1.5 mL) in 5-mL microwave vial. Potassium acetate (39.9 mg, 0.406 mmol), 4,4,4′,4′,5,5,5′,5′-octamethyl-2,2′-bi-1,3,2-dioxaborolane (51.6 mg, 0.203 mmol), and PdCl₂(dppf)-CH₂Cl₂ adduct (8.29 mg, 10.16 umol) were then added with stirring. The vial was purged with nitrogen, sealed, and heated to 100° C. for 2 hours. The reaction mixture was allowed to cool to room temperature. 6-Bromo-1,3-benzothiazole (43.5 mg, 0.203 mmol), PdCl₂(dppf)-CH₂Cl₂ adduct (8.29 mg, 10.16 umol) and 2 M aqueous potassium carbonate (0.305 mL, 0.609 mmol) were then added. The vial was again purged with nitrogen, sealed and heated at 100° C. for 2 hours. The reaction mixture was allowed to cool to room temperature, diluted with water (50 mL), acidified to pH 7 with 1 N HCl, and extracted with DCM (3×50 mL). The combined DCM extracts were dried over sodium sulfate and evaporated to dryness. The resulting crude product was dissolved in DMSO and purified by preparative reverse phase hplc. The appropriate fractions were combined and the pH adjusted to 7 with aqueous sodium bicarbonate (saturated) and extracted with DCM (3×25 mL). The combined DCM extracts were dried over sodium sulfate and evaporated to dryness to afford 43 mg of the titled compound. (LCMS m/z 547.1, M+H).

### Example 149

### 1-{[(3S)-1-(Cyclopropylcarbonyl)-3-pyrrolidinyl]methyl}-2-[4-(1H-pyrrolo[2,3-b]pyridin-5-yl)phenyl]-5-(trifluoromethyl)-1H-benzimidazole

2-(4-Bromophenyl)-1-{[(3S)-1-(cyclopropylcarbonyl)-3-pyrrolidinyl]methyl}-5-(trifluoromethyl)-1H-benzimidazole (100 mg, 0.203 mmol) was dissolved in 1,4-dioxane (1.5 mL) in 5-mL microwave vial. 5-(4,4,5,5-Tetramethyl-1,3,2-dioxaborolan-2-yl)-1H-pyrrolo[2,3-b]pyridine (52.1 mg, 0.213 mmol), PdCl₂(dppf)-CH₂Cl₂ adduct (8.29 mg, 10.16 umol) and 2 M aqueous potassium carbonate (0.305 mL, 0.609 mmol) were then added. The vial was purged with nitrogen, sealed and heated at 100° C. for 2 hours. The reaction mixture was allowed to cool to room temperature, diluted with water (50 mL), acidified to pH 7 with 1 N HCl, and extracted with DCM (3×50 mL). The combined DCM extracts were dried over sodium sulfate and evaporated to dryness. The resulting crude product was dissolved in DMSO and purified by preparative reverse phase hplc. The appropriate fractions were combined and the pH adjusted to 7 with aqueous sodium bicarbonate (saturated) and extracted with DCM (2×25 mL). The combined DCM extracts were dried over sodium sulfate and evaporated to dryness to afford 17 mg of the titled compound. (LCMS m/z 529.9, M+H).

### Example 150

### 1-{[(3S)-1-(Cyclopropylcarbonyl)-3-pyrrolidinyl]methyl}-2-(4-imidazo[1,5-a]pyridin-5-ylphenyl)-5-(trffluoromethyl)-1H-benzimidazole

The titled compound was prepared according to the procedure in Example 148, substituting 5-bromoimidazo[1,5-a]pyridine (40.0 mg, 0.203 mmol). The crude product was purified by preparative reverse phase hplc followed by a second preparative reverse phase hplc purification using a gradient of 1% NH₄OH/acetonitrile. The appropriate fractions were combined and evaporated to dryness to afford 30 mg of the titled compound as a yellowish-green solid. (LCMS, m/z 529.9, M+H).

### Example 151

### 1-{[(3S)-1-(Cyclopropylcarbonyl)-3-pyrrolidinyl]methyl}-2-(4-imidazo[1,2-a]pyridin-5-ylphenyl)-5-(trffluoromethyl)-1H-benzimidazole

The titled compound was prepared according to the procedure in Example 148, substituting 5-bromoimidazo[1,2-a]pyridine (40.0 mg, 0.203 mmol), to afford 52 mg of the titled compound. (LCMS m/z 530.0, M+H).

### Example 152

### 2-[4-(1-Benzofuran-6-yl)phenyl]-1-{[(3S)-1-(cyclopropylcarbonyl)-3-pyrrolidinyl]methyl}-5-(trifluoromethyl)-1H-benzimidazole

The titled compound was prepared according to the procedure in Example 148, substituting 1-benzofuran-6-yl trifluoromethanesulfonate (54.1 mg, 0.203 mmol), to afford 27 mg of the titled compound as an off-white solid. (LCMS, m/z 530.0, M+H).

### Example 153

### 1-{[(3S)-1-(Cyclopropylcarbonyl)-3-pyrrolidinyl]methyl}-2-(4-imidazo[1,2-a]pyridin-3-ylphenyl)-5-(trffluoromethyl)-1H-benzimidazole

2-(4-Bromophenyl)-1-{[(3S)-1-(cyclopropylcarbonyl)-3-pyrrolidinyl]methyl}-5-(trifluoromethyl)-1H-benzimidazole (97 mg, 0.197 mmol) was dissolved in 1,4-dioxane (2 mL) in 5 mL microwave vial. To this was added in bis(pinocolato)diboron (52.5 mg, 0.207 mmol), PdCl₂(dppf)-CH₂Cl₂ adduct (8.04 mg, 9.85 μmmol) and potassium acetate (58.0 mg, 0.591 mmol) with stirring. The vial was purged with nitrogen, sealed and heated to 100° C. for 2 hr. The reaction mixture was allowed to cool to RT and SiliCycle Si-Thiol scavenger resin (20.81 mg, 0.030 mmol) was added in one portion. The vial was again purged with nitrogen, sealed and stirred at RT for 2 hr. The mixture was diluted with EtOAc (20 mL) and filtered through Celite. The Celite was washed with EtOAc (2×20 mL) and the combined EtOAc filtrates were evaporated to dryness. The residue was dissolved in diethyl ether, evaporated to dryness and dried in vacuo overnight to afford 129 mg 1-{[(3S)-1-(cyclopropylcarbonyl)-3-pyrrolidinyl]methyl}-2-[4-(4,4,5,5-tetramethyl-1,3,2-dioxaborolan-2-yl)phenyl]-5-(trifluoromethyl)-1H-benzimidazole as a brown solid, which was used without further purification.

1-{[(3S)-1-(Cyclopropylcarbonyl)-3-pyrrolidinyl]methyl}-2-[4-(4,4,5,5-tetramethyl-1,3,2-dioxaborolan-2-yl)phenyl]-5-(trifluoromethyl)-1H-benzimidazole (129 mg, 0.198 mmol) was dissolved in 1,4-dioxane (1.5 mL) in 5-mL microwave vial. 3-Bromoimidazo[1,2-a]pyridine (39.1 mg, 0.198 mmol), PdCl₂(dppf)-CH₂Cl₂ adduct (8.11 mg, 9.92 umol), and potassium carbonate (0.298 mL, 0.595 mmol) were added with stirring. The vial was purged with nitrogen, sealed and heated at 100° C. for 2 hr. The reaction mixture was allowed to cool to room temperature, diluted with water (50 mL), acidified to pH 7 with 1 N HCl, and extracted with DCM (3×50 mL). The combined DCM extracts were dried over sodium sulfate and evaporated to dryness. The resulting crude product was dissolved in DMSO and purified by preparative reverse phase hplc. The appropriate fractions were combined and the pH adjusted to 7 with aqueous sodium bicarbonate (saturated) and extracted with DCM (2×25 mL). The combined DCM extracts were dried over sodium sulfate and evaporated to dryness to afford 33 mg of the titled compound. (LCMS m/z 530.0, M+H).

### Example 154

### 1-{[(3S)-1-(Cyclopropylcarbonyl)-3-pyrrolidinyl]methyl}-2-[3′-(methylsulfonyl)-4-biphenylyl]-5-(trifluoromethyl)-1H-benzimidazole

The titled compound was prepared according to the procedure in Example 149, substituting [3-(methylsulfonyl)phenyl]boronic acid (40.6 mg, 0.203 mmol) to afford 49 mg of the titled compound as a beige solid. (LCMS m/z 568.3, M+H).

### Example 155

### 1-{[(3S)-1-(Cyclopropylcarbonyl)-3-pyrrolidinyl]methyl}-2-[4′-(methylsulfonyl)-4-biphenylyl]-5-(trifluoromethyl)-1H-benzimidazole

The titled compound was prepared according to the procedure in Example 149, substituting 4,4,5,5-tetramethyl-2-[4-(methylsulfonyl)phenyl]-1,3,2-dioxaborolane (57.3 mg, 0.203 mmol) to afford 50 mg of the titled compound as a beige solid. (LCMS m/z 568.1, M+H).

### Example 156

### 6-{4-[1-{[(3S)-1-(Cyclopropylcarbonyl)-3-pyrrolidinyl]methyl}-5-(trifluoromethyl)-1H-benzimidazol-2-yl]phenyl}-1,3-benzoxazole

### (a) 1-{[(3S)-1-(cyclopropylcarbonyl)-3-pyrrolidinyl]methyl}-2-[4-(4,4,5,5-tetramethyl-1,3,2-dioxaborolan-2-yl)phenyl]-5-(trifluoromethyl)-1H-benzimidazole

2-(4-Bromophenyl)-1-{[(3S)-1-(cyclopropylcarbonyl)-3-pyrrolidinyl]methyl}-5-(trifluoromethyl)-1H-benzimidazole (1 g, 1.726 mmol) was dissolved in 1,4-dioxane (20 mL) in 50 mL pressure vial. Bis(pinocolato)diboron (0.516 g, 2.032 mmol), PdCl₂(dppf)-CH₂Cl₂ adduct (0.083 g, 0.102 mmol) and potassium acetate (480 mg, 4.89 mmol) were added with stirring. The vial was urged with nitrogen, sealed and heated at 100° C. in for 2 hr. The reaction mixture was allowed to cool to RT, and was diluted with ethyl acetate (50 mL). SiliCycle Si-Thiol scavenging resin (225 mg, 0.320 mmol) was added and the mixture was stirred at RT for 1 hr. The reaction mixture was then filtered through Celite and the filtrate was evaporated to dryness to afford 1.48 g of the titled compound, which was used without further purification.

### (b) 6-{4-[1-{[(3S)-1-(Cyclopropylcarbonyl)-3-pyrrolidinyl]methyl}-5-(trifluoromethyl)-1H-benzimidazol-2-yl]phenyl}-1,3-benzoxazole

1-{[(3S)-1-(Cyclopropylcarbonyl)-3-pyrrolidinyl]methyl}-2-[4-(4,4,5,5-tetramethyl-1,3,2-dioxaborolan-2-yl)phenyl]-5-(trifluoromethyl)-1H-benzimidazole (120 mg, 0.167 mmol) was dissolved in 1,4-dioxane (1.5 mL) in 5-mLmicrowave vial. 6-bromo-1,3-benzoxazole (33.0 mg, 0.167 mmol), PdCl₂(dppf)-CH₂Cl₂ adduct (6.81 mg, 8.34 μmol), and 2 M aqueous potassium carbonate (0.250 mL, 0.501 mmol) were added with stirring. The vial was purged with nitrogen, sealed and heated at 100° C. for 2 hr. The reaction mixture was allowed to cool to RT and was diluted with water (50 mL). The pH was adjusted to 7 with 1 N HCl and the mixture was extracted with DCM (3×50 mL). The combined extracts were dried over sodium sulfate, evaporated to dryness, and purified by silica gel column chromatography using a gradient of 0-10% MeOH/DCM, followed by preparative reverse phase hplc using a gradient of 1% aqueous NH₄OH/acetonitrile, to afford 18 mg of the titled compound. (LCMS m/z 531.0, M+H).

### Example 157

### 5-{4-[1-{[(3S)-1-(Cyclopropylcarbonyl)-3-pyrrolidinyl]methyl}-5-(trifluoromethyl)-1H-benzimidazol-2-yl]phenyl}-1,3-dihydro-2H-indol-2-one

The titled compound was prepared according to the procedure in Example 156(b), substituting 5-bromo-1,3-dihydro-2H-indol-2-one (35.4 mg, 0.167 mmol). The crude product was purified by preparative reverse phase hplc. The appropriate fractions were combined, adjusted to pH 7 with saturated aqueous sodium bicarbonate, extracted with DCM (3×25 mL), dried over sodium sulfate and evaporated to dryness to afford 22 mg of the titled compound as an off-white solid. (LCMS m/z 545.2, M+H).

### Example 158

### 1-{[(3S)-1-(Cyclopropylcarbonyl)-3-pyrrolidinyl]methyl}-2-[4-(2,3-dihydro-1H-indol-5-yl)phenyl]-5-(trifluoromethyl)-1H-benzimidazole

1-{[(3S)-1-(Cyclopropylcarbonyl)-3-pyrrolidinyl]methyl}-2-[4-(4,4,5,5-tetramethyl-1,3,2-dioxaborolan-2-yl)phenyl]-5-(trifluoromethyl)-1H-benzimidazole (120 mg, 0.167 mmol) was dissolved in 1,4-dioxane (1.5 mL) in a 5-mL microwave vial. 5-Bromo-2,3-dihydro-1H-indole (33.0 mg, 0.167 mmol), PdCl₂(dppf)-CH₂Cl₂ adduct (6.81 mg, 8.34 umol), and potassium carbonate (0.250 mL, 0.501 mmol) were added with stirring. The vial was purged with nitrogen, sealed and heated at 100° C. for 2 hours. Additional aliquots of (0.076 mmol) of 5-bromo-2,3-dihydro-1H-indole (15 mg, 0.076 mmol) and PdCl₂(dppf)-CH₂Cl₂ adduct (6.81 mg, 8.34 umol) were added and the vial was again purged with nitrogen, sealed, and heated at 100° C. for 2 hr. The reaction mixture was allowed to cool to RT, and was diluted with water (50 mL) and acidified to pH 7 with 1N HCl. The reaction mixture was extracted with DCM (3×50 mL), and the combined DCM layers were dried over MgSO₄, filtered and evaporated to dryness. The crude product was purified by silica gel column chromatography using a gradient of 0-10% MeOH/DCM, followed by preparative reverse phase hplc using a gradient of 1% NH₄OH(aq)/acetonitrile to afford 10 mg of the titled compound as an off-white solid. (LCMS m/z 530.9, M+H).

### Example 159

### 1-{[(3S)-1-(Cyclopropylcarbonyl)-3-pyrrolidinyl]methyl}-2-[4-(1H-pyrrolo[2,3-b]pyridin-6-yl)phenyl]-5-(trifluoromethyl)-1H-benzimidazole

The titled compound was prepared according to the procedure in Example 156b, substituting 6-chloro-1H-pyrrolo[2,3-b]pyridine (25.5 mg, 0.167 mmol). The crude product was purified by preparative reverse phase hplc using a gradient of 1% NH₄OH(aq)/acetonitrile to afford 23 mg of the titled compound as an off-white solid. (LCMS m/z 530.1, M+H).

### Example 160

### 6-{4-[1-{[(3S)-1-(Cyclopropylcarbonyl)-3-pyrrolidinyl]methyl}-5-(trifluoromethyl)-1H-benzimidazol-2-yl]phenyl}-1H-pyrazolo[3,4-b]pyridine

1-{[(3S)-1-(Cyclopropylcarbonyl)-3-pyrrolidinyl]methyl}-2-[4-(4,4,5,5-tetramethyl-1,3,2-dioxaborolan-2-yl)phenyl]-5-(trifluoromethyl)-1H-benzimidazole (120 mg, 0.167 mmol) was dissolved in 1,4-dioxane (1.5 mL) in a 5-mL microwave vial. 6-Chloro-1H-pyrazolo[3,4-b]pyridine (25.6 mg, 0.167 mmol), PdCl₂(dppf)-CH₂Cl₂ adduct (6.81 mg, 8.34 umol), and potassium carbonate (0.250 mL, 0.501 mmol) were added with stirring. The vial was purged with nitrogen, sealed and heated at 100° C. for 2 hours. An additional aliquot of PdCl₂(dppf)-CH₂Cl₂ adduct (6.81 mg, 8.34 umol) were added and the vial was again purged with nitrogen, sealed, and heated at 100° C. overnight. The reaction mixture was allowed to cool to RT, and was diluted with water (50 mL) and acidified to pH 7 with 1N HCl. The reaction mixture was extracted with DCM (3×50 mL), and the combined DCM layers were dried over MgSO₄, filtered and evaporated to dryness. The crude product was purified by silica gel column chromatography using a gradient of 0-10% MeOH/DCM, followed by preparative reverse phase hplc using a gradient of 1% NH₄OH(aq)/acetonitrile to afford 15 mg of the titled compound as an off-white solid. (LCMS m/z 530.9, M+H).

### Example 161

### 2-[4-(1-Benzofuran-3-yl)phenyl]-1-{[(3S)-1-(cyclopropylcarbonyl)-3-pyrrolidinyl]methyl}-5-(trifluoromethyl)-1H-benzimidazole

The titled compound was prepared according to the procedure in Example 160, substituting 3-bromo-1-benzofuran (32.9 mg, 0.167 mmol), to afford 5.0 mg of the titled compound as an off-white solid. (LCMS m/z 530.1, M+H).

### Example 162

### 4′-[1-{[(3S)-1-(Cyclopropylcarbonyl)-3-pyrrolidinyl]methyl}-5-(trifluoromethyl)-1H-benzimidazol-2-yl]-4-biphenylcarbonitrile

The titled compound was prepared according to the procedure in Example 149, substituting (4-cyanophenyl)boronic acid (29.8 mg, 0.203 mmol). The crude product was purified by preparative reverse phase hplc using a gradient of 1% NH₄OH(aq)/acetonitrile to afford 35 mg of the titled compound. (LCMS m/z 515.3, M+H).

### Example 163

### 6-{4-[1-{[(3S)-1-(Cyclopropylcarbonyl)-3-pyrrolidinyl]methyl}-5-(trifluoromethyl)-1H-benzimidazol-2-yl]phenyl}quinazoline

1-{[(3S)-1-(cyclopropylcarbonyl)-3-pyrrolidinyl]methyl}-2-[4-(4,4,5,5-tetramethyl-1,3,2-dioxaborolan-2-yl)phenyl]-5-(trifluoromethyl)-1H-benzimidazole (120 mg, 0.167 mmol) was dissolved in 1,4-dioxane (1.5 mL) in a 5 mL microwave vial. To this was added 6-bromoquinazoline (34.9 mg, 0.167 mmol), PdCl₂(dppf)-CH₂Cl₂ adduct (6.81 mg, 8.34 mmol), and 2.0 M aqueous potassium carbonate (0.250 mL, 0.501 mmol) with stirring. The vial was purged with nitrogen, sealed and heated at 100° C. for six hours. The reaction mixture was allowed to cool and the pH was adjusted to 7 with 1 N HCl. The reaction mixture was extracted with DCM (3×50 mL) and the combined extracts were dried over sodium sulfate, filtered and evaporated to dryness. The crude product was dissolved in DMSO (1.5 mL) and purified by preparative reverse phase HPLC. The appropriate fractions were combined, the pH adjusted to 7 with saturated aqueous NaHCO3, and extracted with DCM (3×25 mL). The combined DCM extracts were dried over sodium sulfate, filtered and evaporated to dryness to afford 29 mg of the titled compound as an off-white solid. (LCMS m/z 542.3, M+H).

### Example 164

### 1-{[(3S)-1-(Cyclopropylcarbonyl)-3-pyrrolidinyl]methyl}-2-[4-(1H-pyrrolo[3,2-c]pyridin-3-yl)phenyl]-5-(trifluoromethyl)-1H-benzimidazole

1-{[(3S)-1-(cyclopropylcarbonyl)-3-pyrrolidinyl]methyl}-2-[4-(4,4,5,5-tetramethyl-1,3,2-dioxaborolan-2-yl)phenyl]-5-(trifluoromethyl)-1H-benzimidazole (120 mg, 0.167 mmol) was dissolved in 1,4-dioxane (1.5 mL) in a 5 mL microwave vial. To this was added 3-bromo-1H-pyrrolo[3,2-c]pyridine (32.9 mg, 0.167 mmol), PdCl₂(dppf)-CH₂Cl₂ adduct (6.81 mg, 8.34 μmol), and 2.0 M aqueous potassium carbonate (0.250 mL, 0.501 mmol) with stirring. The vial was purged with nitrogen, sealed and heated at 100° C. for six hours. An additional aliquot of PdCl₂(dppf)-CH₂Cl₂ adduct (6.81 mg, 8.34 μmmol) was added and the reaction was allowed to stir at 100° C. for two days. Additional aliquots of 3-bromo-1H-pyrrolo[3,2-c]pyridine (11 mg) and PdCl₂(dppf)-CH₂Cl₂ adduct (6.81 mg, 8.34 μmmol) were added and the reaction was allowed to stir overnight at 100° C. The reaction mixture was allowed to cool and the pH was adjusted to 7 with 1 N HCl. The reaction mixture was extracted with DCM (3×50 mL) and the combined extracts were dried over sodium sulfate, filtered and evaporated to dryness. The crude product was purified by silica gel column chromatography using a gradient of 1-10% MeOH/DCM, followed by preparative reverse phase HPLC using a gradient of 1% NH₄OH(aq)/acetonitrile. The appropriate fractions were combined and evaporated to dryness to afford 9 mg of the titled compound as an off-white solid. (LCMS m/z 529.9, M+H).

### Example 165

### 2-[4-(1H-Benzimidazol-5-yl)phenyl]-1-{[(3S)-1-(cyclopropylcarbonyl)-3-pyrrolidinyl]methyl}-5-(trifluoromethyl)-1H-benzimidazole

1-{[(3S)-1-(cyclopropylcarbonyl)-3-pyrrolidinyl]methyl}-2-[4-(4,4,5,5-tetramethyl-1,3,2-dioxaborolan-2-yl)phenyl]-5-(trifluoromethyl)-1H-benzimidazole (120 mg, 0.167 mmol) was dissolved in 1,4-dioxane (1.5 mL) in a 5 mL microwave vial. To this was added 5-bromo-1H-benzimidazole (32.9 mg, 0.167 mmol), PdCl₂(dppf)-CH₂Cl₂ adduct (6.81 mg, 8.34 μmmol), and 2.0 M aqueous potassium carbonate (0.250 mL, 0.501 mmol) with stirring. The vial was purged with nitrogen, sealed and heated at 100° C. for six hours. An additional aliquot of PdCl₂(dppf)-CH₂Cl₂ adduct (6.81 mg, 8.34 μmmol) was added and the reaction was allowed to stir at 100° C. for 2 days. The reaction mixture was allowed to cool and the pH was adjusted to 7 with 1 N HCl. The reaction mixture was extracted with DCM (3×50 mL) and the combined extracts were dried over sodium sulfate, filtered and evaporated to dryness. The crude product was purified by silica gel column chromatography using a gradient of 1-10% MeOH/DCM, followed by preparative reverse phase HPLC using a gradient of 1% NH₄OH(aq)/acetonitrile. The appropriate fractions were combined and evaporated to dryness to afford 15 mg of the titled compound as an off-white solid. (LCMS m/z 530.1, M+H).

### Example 166

### 6-{4-[1-{[(3S)-1-(Cyclopropylcarbonyl)-3-pyrrolidinyl]methyl}-5-(trifluoromethyl)-1H-benzimidazol-2-yl]phenyl}-1(2H)-isoquinolinone

1-{[(3S)-1-(cyclopropylcarbonyl)-3-pyrrolidinyl]methyl}-2-[4-(4,4,5,5-tetramethyl-1,3,2-dioxaborolan-2-yl)phenyl]-5-(trifluoromethyl)-1H-benzimidazole (100 mg, 0.139 mmol) was dissolved in 1,4-dioxane in a 5 mL microwave vial. To this was added 6-bromo-1(2H)-isoquinolinone (31.2 mg, 0.139 mmol), PdC12(dppf)-CH₂Cl₂ adduct (5.68 mg, 6.95 μmmol), and 2.0 M aqueous potassium carbonate (0.209 mL, 0.417 mmol) with stirring. The vial was purged with nitrogen, sealed and heated at 100° C. for 2 hr. The reaction mixture was allowed to cool and the pH was adjusted to 7 with 1 N HCl. The reaction mixture was extracted with DCM (3×50 mL) and the combined extracts were dried over sodium sulfate, filtered and evaporated to dryness. The crude product was dissolved in DMSO (1.5 mL) and purified by preparative reverse phase HPLC using a gradient of 1% NH4OH (aq)/acetonitrile. The appropriate fractions were combined and evaporated to dryness to afford 15 mg of the titled compound as an off-white solid. (LCMS m/z 556.9, M+H).

### Example 167

### 1-{[(3S)-1-(Cyclopropylcarbonyl)-3-pyrrolidinyl]methyl}-2-[4-(2-methyl-1H-indol-5-yl)phenyl]-5-(trifluoromethyl)-1H-benzimidazole

2-(4-Bromophenyl)-1-{[(3S)-1-(cyclopropylcarbonyl)-3-pyrrolidinyl]methyl}-5-(trifluoromethyl)-1H-benzimidazole (246 mg, 0.500 mmol) was dissolved in 1,4-dioxane (2 mL) in a 5 mL microwave vial. To this was added potassium acetate (98 mg, 0.999 mmol), 4,4,4′,4′,5,5,5′,5′-octamethyl-2,2′-bi-1,3,2-dioxaborolane (127 mg, 0.500 mmol), and PdCl₂(dppf)-CH₂Cl₂ adduct (20.40 mg, 0.025 mmol), and 5-bromo-2-methyl-1H-indole (105 mg, 0.500 mmol). The vial was purged with nitrogen, sealed, and heated at 100° C. for 2 hr. Additional aliquots of 5-bromo-2-methyl-1H-indole (105 mg, 0.500 mmol), PdCl₂(dppf)-CH₂Cl₂ adduct (20.40 mg, 0.025 mmol) and 2.0 M aqueous potassium carbonate (0.749 mL, 1.499 mmol) were added and the reaction was allowed to stir at 100° C. overnight. The reaction mixture was allowed to cool to RT and was diluted with water (50 mL) and extracted with DCM (3×50 mL). The combined DCM extracts were dried over sodium sulfate, filtered and evaporated to dryness. The crude product was purified by silica gel column chromatography using a gradient of 0-8% MeOH/DCM, followed by preparative reverse phase HPLC. The appropriate fractions were combined, the pH adjusted to 7 with saturated aqueous NaHCO3, and extracted with DCM (3×25 mL). The combined DCM extracts were dried over sodium sulfate, filtered and evaporated to dryness to afford 21 mg of the titled compound as an off-white solid. (LCMS m/z 543.2, M+H).

### Example 168

### 2-[4-(1-Benzofuran-5-yl)phenyl]-1-{[(3S)-1-(cyclopropylcarbonyl)-3-pyrrolidinyl]methyl}-1H-benzimidazole-5-carbonitrile

### (a) 4-Chloro-3-nitrobenzonitrile

A suspension of 4-chloro-3-nitrobenzoic acid (5 g, 24.81 mmol) and oxalyl chloride (2.309 mL, 27.3 mmol) in DCM (50 mL) was treated with N,N-dimethylformamide (0.25 ml) and stirred at room temperature for 2 hours, until a clear solution resulted. The mixture was evaporated, redissolved in DCM (4 mL) and added to a cold solution of 7 M ammonia in methanol (6 mL) in chloroform (10 mL) in an ice bath. Solvent was evaporated under reduced pressure and the residue azeotroped successively with ethanol and chloroform to give the 4-chloro-3-nitrobenzamide as an off white solid.

A mixture of phosphorus pentoxide (20 g, 139 mmol) and hexamethyldisiloxane (50.0 mL, 235 mmol) in chloroform (100 mL) was heated under reflux in an argon atmosphere for 2 hours. The 4-chloro-3-nitrobenzamide was added to this solution and the mixture was heated under reflux for 2 hours. Most of the solvent was removed under reduced pressure and the oily residue applied to a pad of silica gel in a sintered funnel and washed through with hexanes (600 mL), the receiving flask changed and the pad washed with 5% methanol in dichloromethane (2×400 mL). The combined dichloromethane solutions were evaporated and the residue purified by silica gel column chromatography (dichloromethane then 5% methanol in dichloromethane) to give the title compound 4-chloro-3-nitrobenzonitrile (3.26 g, 17.86 mmol, 72.0% yield). 1H NMR (400 MHz, chloroform-d) δ ppm 7.75 (d, 1H) 7.83 (dd, 1H) 8.21 (d, J=1.77 Hz, 1H). The product contained about 25% of the methyl benzoate impurity and was taken on to the next step without further purification.

### (b) 4-({[(3S)-1-(Cyclopropylcarbonyl)-3-pyrrolidinyl]methyl}amino)-3-nitrobenzonitrile

4-Chloro-3-nitrobenzonitrile (560 mg, 3.33 mmol), {[(3S)-1-(cyclopropylcarbonyl)-3-pyrrolidinyl]methyl}amine (729 mg, 3.99 mmol), and DIEA (1.739 mL, 9.99 mmol) were dissolved in ethanol (50 mL) with stirring under nitrogen, and allowed to stir at 75° C. for 4 hours. The reaction mixture was evaporated, and the residue taken up in DCM and washed with water. The aqueous layer was extracted with DCM and the combined organics were washed with saturated brine, dried over sodium sulfate and evaporated to give 4-({[(3S)-1-(cyclopropylcarbonyl)-3-pyrrolidinyl]methyl}amino)-3-nitrobenzonitrile (900 mg, 2.86 mmol, 86% yield). 1H NMR (400 MHz, chloroform-d) δ ppm 0.77-0.84 (m, 2H) 0.99-1.07 (m, 2H) 1.53-1.68 (m, 2H) 1.72-1.98 (m, 1H) 2.12-2.47 (m, 1H) 2.56-2.95 (m, 1H) 3.24-3.65 (m, 3H) 3.66-3.79 (m, 1H) 3.79-3.92 (m, 1H) 6.84-7.06 (m, 1H) 7.54-7.72 (m, 1H) 8.55 (td, J=4.04, 2.02 Hz, 2H). The material was carried on to the next step without further purification.

### (c) 3-Amino-4-({[(3S)-1-(cyclopropylcarbonyl)-3-pyrrolidinyl]methyl}amino)-benzonitrile

A mixture of 4-({[(3S)-1-(cyclopropylcarbonyl)-3-pyrrolidinyl]methyl}amino)-3-nitrobenzonitrile (900 mg, 2.86 mmol) and 10% Pd/C (500 mg, 2.86 mmol) were suspended in ethanol (50 mL) and stirred vigorously under a hydrogen atmosphere for 2 hours. The mixture was filtered through celite, washed through with ethanol and the cake doused with water. The ethanolic solution was evaporated to a foam to give 3-amino-4-({[(3S)-1-(cyclopropylcarbonyl)-3-pyrrolidinyl]methyl}amino)benzonitrile (710 mg, 2.497 mmol, 87% yield). 1H NMR (400 MHz, chloroform-d) δ ppm 0.74-0.85 (m, 2H) 0.97-1.08 (m, 2H) 1.56-1.69 (m, 1H) 1.69-1.94 (m, 1H) 2.04-2.31 (m, 1H) 2.48-2.76 (m, 1H) 3.12-3.38 (m, 3H) 3.39-3.57 (m, 1H) 3.63-3.90 (m, 4H) 6.60 (dd, J=11.37, 8.34 Hz, 1H) 6.93-7.01 (m, 1H) 7.10-7.23 (m, 1H). The material was carried on to the next step without further purification.

### (d) 2-(4-Bromophenyl)-1-{[(3S)-1-(cyclopropylcarbonyl)-3-pyrrolidinyl]methyl}-1H-benzimidazole-5-carbonitrile

A solution of 3-amino-4-({[(3S)-1-(cyclopropylcarbonyl)-3-pyrrolidinyl]methyl}amino)-benzonitrile (700 mg, 2.462 mmol) and 4-bromobenzaldehyde (729 mg, 3.94 mmol) in 1-butanol (100 ml) was strirred at 80° C., with air being bubbled through the hot solution, for 2 hours. The mixture was cooled and evaporated to a red oil, which was purified by silica gel column chromatography (dichloromethane, then to 5% methanol in dichloromethane over 15 minutes) to give 2-(4-bromophenyl)-1-{[(3S)-1-(cyclopropylcarbonyl)-3-pyrrolidinyl]methyl}-1H-benzimidazole-5-carbonitrile (730 mg, 1.625 mmol, 66.0% yield). 1H NMR (400 MHz, chloroform-d) δ ppm 0.66-0.86 (m, 2H) 0.90-1.01 (m, 3H) 1.31-1.62 (m, 4H) 1.77-1.97 (m, 1H) 2.52-2.83 (m, 1 H) 2.92-3.26 (m, 1H) 3.40-3.60 (m, 2H) 4.21-4.52 (m, 2H) 7.52 (d, J=8.59 Hz, 1H) 7.57-7.68 (m, 3H) 7.69-7.80 (m, 2H) 8.18 (dd, J=3.92, 0.88 Hz, 1H).

### (e) 2-[4-(1-Benzofuran-5-yl)phenyl]-1-{[(3S)-1-(cyclopropylcarbonyl)-3-pyrrolidinyl]methyl}-1H-benzimidazole-5-carbonitrile

A mixture of 2-(4-bromophenyl)-1-{[(3S)-1-(cyclopropylcarbonyl)-3-pyrrolidinyl]methyl}-1H-benzimidazole-5-carbonitrile (120 mg, 0.267 mmol), 5-(4,4,5,5-tetramethyl-1,3,2-dioxaborolan-2-yl)-1-benzofuran (78 mg, 0.320 mmol), potassium carbonate (148 mg, 1.068 mmol) and tetrakis(triphenylphosphine)palladium(0) (15.43 mg, 0.013 mmol) in 1,4-dioxane (5 ml) and water (2 ml) was sealed in a microwave vessel and heated at 110° C. for 2 hr. The mixture was partitioned between ethyl acetate and dilute brine. The aqueous layer was separated and extracted with ethyl acetate, and the combined extracts washed with brine and evaporated. The crude product was purified by silica gel column chromatography (dichloromethane, then 3.5% methanol in dichloromethane) followed by preparative reverse phase HPLC to afford 40 mg of the titled compound (0.082 mmol, 30.8% yield) as a white solid. (LCMS m/z 487.4, M+H).

### Example 169

### 1-{[(3S)-1-(Cyclopropylcarbonyl)-3-pyrrolidinyl]methyl}-2-[4-(1H-indol-5-yl)phenyl]-1H-benzimidazole-5-carbonitrile

The titled compound was prepared according to the procedure in Example 168d, substituting 5-(4,4,5,5-tetramethyl-1,3,2-dioxaborolan-2-yl)-1H-indole (64.9 mg, 0.267 mmol). The crude product was purified by preparative reverse phase HPLC to afford 85 mg of the titled compound as a white solid. (LCMS m/z 486.3, M+H).

### Example 170

### 2-[4-(1-Benzofuran-5-yl)phenyl]-1-{[(3S)-1-(cyclopropylcarbonyl)-3-pyrrolidinyl]methyl}-1H-benzimidazole-6-carbonitrile

### (a) 3-({[(3S)-1-(Cyclopropylcarbonyl)-3-pyrrolidinyl]methyl}amino)-4-nitrobenzonitrile

A mixture of {[(3S)-1-(cyclopropylcarbonyl)-3-pyrrolidinyl]methyl}amine (495 mg, 2.94 mmol), 3-chloro-4-nitrobenzonitrile (537 mg, 2.94 mmol), and DIEA (1.025 mL, 5.88 mmol) were dissolved in 1,4-dioxane (10 mL) in a microwave vial. The solution was heated at 150° C. for 2 hours. The reaction mixture was evaporated, taken up in DCM and washed with water. The aqueous wash was extracted with DCM and the combined organics evaporated and purified by silica gel column chromatography using a gradient of 0-5% MeOH/DCM to afford the titled compound (467 mg, 1.486 mmol, 50.5% yield). ¹H NMR (400 MHz, chloroform-d) δ ppm 0.71-0.89 (m, 2H) 0.95-1.10 (m, 2H) 1.62-1.69 (m, 1H) 1.70-2.00 (m, 1H) 2.12-2.42 (m, 1H) 2.54-2.91 (m, 1H) 3.23-4.04 (m, 6H) 6.95 (td, J=8.65, 1.64 Hz, 1H) 7.18 (dd, J=8.34, 1.52 Hz, 1H) 8.01-8.20 (m, 1H) 8.30 (dd, J=8.72, 5.68 Hz, 1H).

### (b) 4-Amino-3-({[(3S)-1-(cyclopropylcarbonyl)-3-pyrrolidinyl]methyl}amino)-benzonitrile

A mixture of 3-({[(3S)-1-(cyclopropylcarbonyl)-3-pyrrolidinyl]methyl}amino)-4-nitrobenzonitrile (880 mg, 2.80 mmol) and 10% Pd/C (500 mg, 2.86 mmol) were suspended in ethanol (50 mL) and stirred vigorously under a hydrogen atmosphere for 4 hours. The mixture was filtered through a PTFE filter, washed with ethanol and the cake doused with water. The ethanolic filtrate was evaporated to afford the titled compound (750 mg, 2.64 mmol, 92% yield). 1H NMR (400 MHz, chloroform-d) δ ppm 0.76-0.83 (m, 2H) 0.99-1.05 (m, 2H) 1.62-1.69 (m, 3H) 1.72-1.95 (m, 1H) 2.11-2.32 (m, 1H) 2.49-2.77 (m, 1H) 3.24-3.97 (m, 7H) 6.67-6.76 (m, 1H) 6.88 (dd, J=12.25, 1.64 Hz, 1H) 7.01-7.12 (m, 1H).

### (c) 2-(4-Bromophenyl)-1-{[(3S)-1-(cyclopropylcarbonyl)-3-pyrrolidinyl]methyl}-1H-benzimidazole-6-carbonitrile

A solution of 4-amino-3-({[(3S)-1-(cyclopropylcarbonyl)-3-pyrrolidinyl]methyl}amino)benzonitrile (700 mg, 2.462 mmol) and 4-bromobenzaldehyde (729 mg, 3.94 mmol) in 1-butanol (100 ml) was strirred at 80° C., with air being bubbled through the hot solution, for 2 hours. The mixture was cooled and evaporated to a red oil, which was purified by silica gel column chromatography, eluting with a DCM, then a gradient of 0-5% MeOH/DCM to afford the titled compound (913 mg, 2.032 mmol, 83% yield). 1H NMR (400 MHz, chloroform-d) δ ppm 0.67-0.84 (m, 2H) 0.88-1.10 (m, 2H) 1.31-1.58 (m, 2H) 1.76-2.01 (m, 1H) 2.56-2.84 (m, 1H) 2.99-3.21 (m, 1H) 3.22-3.45 (m, 1H) 3.45-3.63 (m, 2H) 4.29-4.49 (m, 2H) 7.57-7.68 (m, 3H) 7.69-7.77 (m, 2H) 7.79 (dd, J=4.04, 0.76 Hz, 1H) 7.86-8.00 (m, 1H).

### (d) 2-[4-(1-Benzofuran-5-yl)phenyl]-1-{[(3S)-1-(cyclopropylcarbonyl)-3-pyrrolidinyl]methyl}-1H-benzimidazole-6-carbonitrile

A mixture of 2-(4-bromophenyl)-1-{[(3S)-1-(cyclopropylcarbonyl)-3-pyrrolidinyl]methyl}-1H-benzimidazole-6-carbonitrile (120 mg, 0.267 mmol), 5-(4,4,5,5-tetramethyl-1,3,2-dioxaborolan-2-yl)-1-benzofuran (78 mg, 0.320 mmol), potassium carbonate (148 mg, 1.068 mmol) and tetrakis(riphenylphosphine)palladium(0) (15.43 mg, 0.013 mmol) in 1,4-dioxane (5 ml) and water (2 ml) was sealed in a microwave vial and heated at 120° C. for 2 hr. The mixture was partitioned between EtOAc and dilute brine. The brine layer was extracted with EtOAc and the combined extracts washed with brine and evaporated. The crude product was purified by preparative reverse phase HPLC to afford 80 mg of the titled compound. (LCMS m/z 487.3, M+H).

### Example 171

### 1-{[(3S)-1-(Cyclopropylcarbonyl)-3-pyrrolidinyl]methyl}-2-[4-(1H-indol-5-yl)phenyl]-1H-benzimidazole-6-carbonitrile

A mixture of 2-(4-bromophenyl)-1-{[(3S)-1-(cyclopropylcarbonyl)-3-pyrrolidinyl]methyl}-1H-benzimidazole-6-carbonitrile (120 mg, 0.267 mmol), 5-(4,4,5,5-tetramethyl-1,3,2-dioxaborolan-2-yl)-1H-indole (78 mg, 0.320 mmol), potassium carbonate (148 mg, 1.068 mmol) and tetrakis(riphenylphosphine)palladium(0) (15.43 mg, 0.013 mmol) in 1,4-dioxane (5 ml) and water (2 ml) was sealed in a microwave vial and heated at 110° C. for 2 hr. The mixture was partitioned between EtOAc and dilute brine. The brine layer was extracted with EtOAc and the combined extracts washed with brine and evaporated. The crude product was purified by preparative reverse phase HPLC to afford 50 mg of the titled compound. (LCMS m/z 486.3, M+H).

### Example 172

### 1-{[(3S)-1-(Cyclopropylcarbonyl)-3-pyrrolidinyl]methyl}-2-[4-(1H-indol-6-yl)phenyl]-1H-benzimidazole-6-carbonitrile

The titled compound was prepared according to the procedure in Example 171, substituting 1H-indol-6-ylboronic acid (43.0 mg, 0.267 mmol), to afford 60 mg of the titled compound as a yellow solid. (LCMS m/z 486.3, M+H).

### Example 173

### 2-[4-(1-Benzofuran-5-yl)phenyl]-1-{[(3S)-1-(cyclopropylcarbonyl)-3-pyrrolidinyl]methyl}-1H-benzimidazole-7-carbonitrile

### (a) 2-({[(3S)-1-(Cyclopropylcarbonyl)-3-pyrrolidinyl]methyl}amino)-3-nitrobenzonitrile

A mixture of {[(3S)-1-(cyclopropylcarbonyl)-3-pyrrolidinyl]methyl}amine (553 mg, 3.29 mmol), 2-chloro-3-nitrobenzonitrile (500 mg, 2.74 mmol), and DIEA (1.431 mL, 8.22 mmol) were dissolved in 1,4-dioxane (10 mL) in a microwave Vial. The mixture was heated at 150° C. for 1 hour. The mixture was evaporated, taken up in DCM and washed with water. The aqueous layer was extracted with DCM and the combined organics evaporated and purified by silica gel column chromatography using a gradient of 0-5% MeOH/DCM to afford the titled compound (800 mg, 2.55 mmol, 93% yield) as an orange solid. 1H NMR (400 MHz, chloroform-d) δ ppm 0.71-0.88 (m, 2H) 0.94-1.12 (m, 2H) 1.59-1.69 (m, 1H) 1.69-2.00 (m, 1H) 2.11-2.41 (m, 1H) 2.55-2.92 (m, 0H) 3.16-4.11 (m, 6H) 6.80 (ddd, J=10.04, 8.40, 7.58 Hz, 1H) 7.79 (ddd, J=9.35, 7.71, 1.39 Hz, 1H) 8.27-8.49 (m, 1H) 8.53-8.80 (m, 1H).

### (b) 3-Amino-2-({[(3S)-1-(cyclopropylcarbonyl)-3-pyrrolidinyl]methyl}amino)-benzonitrile

A mixture of 2-({[(3S)-1-(cyclopropylcarbonyl)-3-pyrrolidinyl]methyl}amino)-3-nitrobenzonitrile (800 mg, 2.55 mmol) and 10% Pd/C (500 mg, 2.86 mmol) were suspended in ethanol (50 mL) and stirred vigorously under a hydrogen atmosphere for 2 hours. The mixture was filtered through a PTFE filter, washed through with ethanol and the cake doused with water. The ethanolic filtrate was evaporated to a gum to give 3-amino-2-({[(3S)-1-(cyclopropylcarbonyl)-3-pyrrolidinyl]methyl}amino)benzonitrile (674 mg, 2.370 mmol, 83% yield), which was used without further purification. 1H NMR (400 MHz, chloroform-d) δ ppm 0.73-0.83 (m, 2H) 0.94-1.09 (m, 2H) 1.58-1.91 (m, 3H) 2.07-2.36 (m, 1H) 2.39-2.67 (m, 1H) 3.18-3.36 (m, 2H) 3.38-3.51 (m, 1H) 3.59-3.71 (m, 2H) 3.77-4.00 (m, 3H) 6.84-6.94 (m, 2H) 6.96-7.04 (m, 1H).

### (c) 2-(4-Bromophenyl)-1-{[(3S)-1-(cyclopropylcarbonyl)-3-pyrrolidinyl]methyl}-1H-benzimidazole-7-carbonitrile

A solution of 3-amino-2-({[(3S)-1-(cyclopropylcarbonyl)-3-pyrrolidinyl]methyl}amino)-benzonitrile (660 mg, 2.321 mmol) and 4-bromobenzaldehyde (687 mg, 3.71 mmol) in 1-butanol (100 ml) was strirred at 80° C., with air being bubbled through the hot solution, for 2 hours. The mixture was cooled and evaporated to a red oil, which was purified by silica gel column chromatography using DCM and then a gradient of 0-5% MeOH/DCM to afford the titled compound (910 mg, 2.025 mmol, 87% yield). 1H NMR (400 MHz, chloroform-d) δ ppm 0.65-0.79 (m, 2H) 0.85-1.00 (m, 2H) 1.31-1.64 (m, 2H) 1.73-2.06 (m, 1H) 2.62-2.93 (m, 1H) 3.06-3.33 (m, 1H) 3.35-3.45 (m, 1H) 3.46-3.73 (m, 2H) 4.54-4.86 (m, 2H) 7.42 (dt, J=9.85, 7.96 Hz, 1H) 7.59 (dd, J=8.59, 2.27 Hz, 2H) 7.64-7.81 (m, 3H) 8.02-8.13 (m, 1H).

### (d) 2-[4-(1-Benzofuran-5-yl)phenyl]-1-{[(3S)-1-(cyclopropylcarbonyl)-3-pyrrolidinyl]methyl}-1H-benzimidazole-7-carbonitrile

A mixture of 2-(4-bromophenyl)-1-{[(3S)-1-(cyclopropylcarbonyl)-3-pyrrolidinyl]methyl}-1H-benzimidazole-7-carbonitrile (120 mg, 0.267 mmol), 5-(4,4,5,5-tetramethyl-1,3,2-dioxaborolan-2-yl)-1-benzofuran (78 mg, 0.320 mmol), potassium carbonate (148 mg, 1.068 mmol) and tetrakis(riphenylphosphine)palladium(0) (15.43 mg, 0.013 mmol) in 1,4-dioxane (5 ml) and water (2 ml) was sealed in a microwave vial and heated at 110° C. for 2 hr. The mixture was partitioned between EtOAc and dilute brine. The brine layer was extracted with EtOAc and the combined extracts washed with dilute brine and evaporated. The crude product was purified by preparative reverse phase HPLC to afford 90 mg of the titled compound. (LCMS m/z 487.3, M+H).

### Example 174

### 1-{[(3S)-1-(Cyclopropylcarbonyl)-3-pyrrolidinyl]methyl}-2-[4-(1H-indol-6-yl)phenyl]-1H-benzimidazole-7-carbonitrile

The titled compound was prepared according to the procedure in Example 173d, substituting 1H-indol-6-ylboronic acid (43.0 mg, 0.267 mmol), to afford 70 mg of the intended product. (LCMS m/z 486.3, M+H).

### Example 175

### 1-{[(3S)-1-(cyclopropylcarbonyl)-3-pyrrolidinyl]methyl}-2-[4-(1H-indol-5-yl)phenyl]-1H-benzimidazole-7-carbonitrile

The titled compound was prepared according to the procedure in Example 173d, substituting 5-(4,4,5,5-tetramethyl-1,3,2-dioxaborolan-2-yl)-1H-indole (78 mg, 0.320 mmol), to afford 81 mg of the intended product. (LCMS m/z 486.3, M+H).

### Example 176

### N′-[4′-(7-cyano-1-{[(3S)-1-(cyclopropylcarbonyl)-3-pyrrolidinyl]methyl}-1H -benzimidazol-2-yl)-3-biphenylyl]-N,N-dimethylsulfamide

A mixture of 2-(4-bromophenyl)-1-{[(3S)-1-(cyclopropylcarbonyl)-3-pyrrolidinyl]methyl}-1H-benzimidazole-7-carbonitrile (120 mg, 0.267 mmol), (3-{[(dimethylamino)sulfonyl]amino}phenyl)boronic acid (65.2 mg, 0.267 mmol), potassium carbonate (148 mg, 1.068 mmol) and tetrakis(riphenylphosphine)palladium(0) (15.43 mg, 0.013 mmol) in 1,4-dioxane (5 ml) and water (2 ml) was sealed in a microwave vial and heated at 120° C. for 2 hr. The mixture was partitioned between EtOAc and dilute brine. The brine layer was extracted with EtOAc and the combined extracts washed with brine and evaporated. The crude product was purified by preparative reverse phase HPLC to afford 90 mg of the titled compound. (LCMS m/z 569.2, M+H).

### Example 177

### 2-[4-(1-Benzofuran-5-yl)phenyl]-1-{[(3S)-1-(cyclopropylcarbonyl)-3-pyrrolidinyl]methyl}-1H-benzimidazole-4-carbonitrile

### (a) 3-({[(3S)-1-(Cyclopropylcarbonyl)-3-pyrrolidinyl]methyl}amino)-2-nitrobenzonitrile

A mixture of {[(3S)-1-(cyclopropylcarbonyl)-3-pyrrolidinyl]methyl}amine (539 mg, 3.20 mmol), 3-chloro-2-nitrobenzonitrile (487 mg, 2.67 mmol), and DIEA (1.4 mL, 8.04 mmol) were dissolved in 1,4-dioxane (10 mL) in a microwave vial and heated at 150° C. for 2 hours. The reaction mixture was evaporated, taken up in DCM and washed with water. The aqueous layer was extracted with DCM and the combined organics evaporated and purified by silica gel column chromatography using a gradient of 0-5% MeOH/DCM to afford the titled compound (650 mg, 2.068 mmol, 77% yield), which was used without further purification.

### (b) 2-amino-3-({[(3S)-1-(Cyclopropylcarbonyl)-3-pyrrolidinyl]methyl}amino)-benzonitrile

A mixture of 3-({[(3S)-1-(cyclopropylcarbonyl)-3-pyrrolidinyl]methyl}amino)-2-nitrobenzonitrile (900 mg, 2.86 mmol) and 10% Pd/C (500 mg, 2.86 mmol) were suspended in ethanol (50 mL) and stirred vigorously under a hydrogen atmosphere for 2 hr. The mixture was filtered through a PTFE filter, washed through with ethanol and the cake doused with water. The ethanolic filtrate was evaporated to afford the titled compound (660 mg, 2.321 mmol, 81% yield), which was used without further purification. 1H NMR (400 MHz, chloroform-d) δ ppm 0.63-0.84 (m, 2H) 0.93-1.13 (m, 2H) 1.42-1.68 (m, 1H) 1.67-1.95 (m, 1H) 2.00-2.36 (m, 1H) 2.41-2.75 (m, 1H) 3.01-3.59 (m, 3H) 3.75-4.06 (m, 2H) 6.53-7.07 (m, 2H) 7.34-7.69 (m, 1H).

### (c) 2-(4-Bromophenyl)-1-{[(3S)-1-(cyclopropylcarbonyl)-3-pyrrolidinyl]methyl}-1H-benzimidazole-4-carbonitrile

A solution of 2-amino-3-({[(3S)-1-(cyclopropylcarbonyl)-3-pyrrolidinyl]methyl}amino)benzonitrile (660 mg, 2.321 mmol) and 4-bromobenzaldehyde (687 mg, 3.71 mmol) in 1-butanol (100 ml) was stirred at 80° C., with air being bubbled through the hot solution, for 2 hours. The mixture was cooled and evaporated to a brown oil, which was purified by silica gel column chromatography using 70% EtOAc/hexanes followed by 5% MeOH/DCM to afford the titled compound, (306 mg, 0.681 mmol, 29.3% yield).

### (d) 2-[4-(1-Benzofuran-5-yl)phenyl]-1-{[(3S)-1-(cyclopropylcarbonyl)-3-pyrrolidinyl]methyl}-1H-benzimidazole-4-carbonitrile

A mixture of 2-(4-bromophenyl)-1-{[(3S)-1-(cyclopropylcarbonyl)-3-pyrrolidinyl]methyl}-1H-benzimidazole-4-carbonitrile (100 mg, 0.223 mmol), 5-(4,4,5,5-tetramethyl-1,3,2-dioxaborolan-2-yl)-1-benzofuran (65.2 mg, 0.267 mmol), potassium carbonate (123 mg, 0.890 mmol) and tetrakis(riphenylphosphine)palladium(0) (12.86 mg, 0.011 mmol) in 1,4-dioxane (5 ml) and water (2 ml) was sealed in a microwave vial and heated at 110° C. for 2 hr. The mixture was partitioned between EtOAc and dilute brine. The brine layer was extracted with EtOAc and the combined extracts washed with brine and evaporated. The crude product was purified by silica gel column chromatography using a gradient of 0-3.5% MedoH/DCM followed by preparative reverse phase HPLC to afford 52 mg of the titled compound. (LCMS m/z 487.3, M+H).

### Example 178

### 1-{[(3S)-1-(Cyclopropylcarbonyl)-3-pyrrolidinyl]methyl}-2-[4-(1H-indol-5-yl)phenyl]-1H-benzimidazole-4-carbonitrile

A mixture of 2-(4-bromophenyl)-1-{[(3S)-1-(cyclopropylcarbonyl)-3-pyrrolidinyl]methyl}-1H-benzimidazole-4-carbonitrile (90 mg, 0.200 mmol), 5-(4,4,5,5-tetramethyl-1,3,2-dioxaborolan-2-yl)-1H-indole (58.4 mg, 0.240 mmol), potassium carbonate (111 mg, 0.801 mmol) and yetrakis(riphenylphosphine)palladium(0) (11.57 mg, 10.01 μmmol) in 1,4-dioxane (5 ml) and water (2 ml) was sealed in a microwave vial and heated at 110° C. for 2 hr. The reaction mixture was allowed to cool and then partitioned between EtOAc and dilute brine. The brine layer was extracted with EtOAc and the combined extracts washed with brine and evaporated. The crude product was purified by preparative reverse phase HPLC to afford 37 mg of the titled compound. (LCMS m/z 486.3, M+H).

### Example 179

### 1-{[(3S)-1-(cyclopropylcarbonyl)-3-pyrrolidinyl]methyl}-2-[4-(1H-indol-6-yl)phenyl]-1H-benzimidazole-4-carbonitrile

A mixture of 2-(4-bromophenyl)-1-{[(3S)-1-(cyclopropylcarbonyl)-3-pyrrolidinyl]methyl}-1H-benzimidazole-4-carbonitrile (100 mg, 0.223 mmol), 1H-indol-6-ylboronic acid (35.8 mg, 0.223 mmol), potassium carbonate (123 mg, 0.890 mmol) and Tetrakis(triphenylphosphine)palladium(0) (12.86 mg, 0.011 mmol) in 1,4-dioxane (5 ml) and water (2 ml) was sealed in a microwave vial and heated at 120° C. for 2 hr. The reaction mixture was allowed to cool and then partitioned between EtOAc and water. The aqueous layer was extracted with EtOAc and the combined extracts washed with brine and evaporated. The crude product was purified by preparative reverse phase HPLC to afford 52 mg of the titled compound as a yellow solid. (LCMS m/z 486.3, M+H).

### Example 180

### 5-[4-(1-{[(3S)-1-(Cyclopropylcarbonyl)-3-pyrrolidinyl]methyl}-6-fluoro-1H-benzimidazol-2-yl)phenyl]-1H-indazole

### (a) 1,1-Dimethylethyl (3S)-3-{[(5-fluoro-2-nitrophenyl)amino]methyl}-1-pyrrolidinecarboxylate

2-Chloro-4-fluoro-1-nitrobenzene (1.8 g, 10.25 mmol). 1,1-dimethylethyl (3S)-3-(aminomethyl)-1-pyrrolidinecarboxylate (2.7 g, 13.48 mmol) Cs₂CO₃ (4.8 g, 14.73 mmol), Pd(OAc)₂ (0.126 g, 0.561 mmol) and BINAP (0.59 g, 0.948 mmol) were suspended in toluene (16 mL) in a microwave vial under nitrogen. The reaction mixture was heated in a microwave reactor at 100° C. for 45 min. An additional aliquot Pd Pd(OAc)₂ (˜100 mg) was added and the reaction heated an additional 10 min in the microwave reactor. The reaction mixture was filter and partitioned between EtOAc and aqueous NaHCO3. The EtOAc layer was washed with brine, dry over sodium sulfate, filtered, and evaporated to dryness. The crude 1,1-dimethylethyl (3S)-3-{[(5-fluoro-2-nitrophenyl)amino]methyl}-1-pyrrolidinecarboxylate was purified by silica gel column chromatography using a gradient of 0-10% IPA/EtOAc and used without further characterization.

### (b) (5-fluoro-2-nitrophenyl)[(3R)-3-pyrrolidinylmethyl]amine hydrochloride

1,1-dimethylethyl (3S)-3-{[(5-fluoro-2-nitrophenyl)amino]methyl}-1-pyrrolidinecarboxylate (3.5 g) was dissolved in MeOH (30 mL) and treated with 4 N HCl/dioxane (12.89 mL) at RT overnight. The solvent was removed under reduced pressure to afford (5-fluoro-2-nitrophenyl)[(3R)-3-pyrrolidinylmethyl]amine hydrochloride which was taken on to the next step without further purification.

### (c) {[(3S)-1-(Cyclopropylcarbonyl)-3-pyrrolidinyl]methyl}(5-fluoro-2-nitrophenyl)amine

(5-Fluoro-2-nitrophenyl)[(3R)-3-pyrrolidinylmethyl]amine hydrochloride (2.5 g, 9.07 mmol) was dissolved in THF (40 mL) under nitrogen. DIEA (7.92 ml, 45.3 mmol) was added and the reaction mixture allowed to stir for 30 min. Cyclopropanecarbonyl chloride (1.04 g, 9.97 mmol) was then added slowly and the reaction mixture allowed to stir at RT for 1 hr. Solvent was removed under reduced pressure and the residue was dissolved in EtOAc, washed with aqueous NaHCO3, dried over sodium sulfate, filtered and evaporated to dryness. The crude {[(3S)-1-(cyclopropylcarbonyl)-3-pyrrolidinyl]methyl}(5-fluoro-2-nitrophenyl)amine was purified by silica gel column chromatography using a gradient of 0-10% IPA/EtOAc and taken on to the next step without further purification.

### (d) N2-{[(3S)-1-(cyclopropylcarbonyl)-3-pyrrolidinyl]methyl}-4-fluoro-1,2-benzenediamine

{[(3S)-1-(cyclopropylcarbonyl)-3-pyrrolidinyl]methyl}(5-fluoro-2-nitrophenyl)amine (2.5 g, 8.13 mmol) was stirred over 10% Pd/C (0.866 g) in 50 mL EtOH in a hydrogen atmosphere at RT overnight. The reaction mixture was filtered and the filtrate evaporated to dryness to afford N2-{[(3S)-1-(cyclopropylcarbonyl)-3-pyrrolidinyl]methyl}-4-fluoro-1,2-benzenediamine, which was used without further purification.

### (e) 2-(4-Bromophenyl)-1-{[(3S)-1-(cyclopropylcarbonyl)-3-pyrrolidinyl]methyl}-6-fluoro-1H-benzimidazole

N2-{[(3S)-1-(cyclopropylcarbonyl)-3-pyrrolidinyl]methyl}-4-fluoro-1,2-benzenediamine (7.03 mmol) and 4-bromobenzaldehyde (1.561 g, 8.44 mmol) were added to a microwave vial under nitrogen. 1-Butanol (20 mL) was added and the reaction mixture was heated in a microwave reaction at 100° C. for 1 hr. An aliquot of 10% Pd/C (˜100 mg) was added to the reaction mixture and it was heated a conventionally at 100° C. for 1 hour. The reaction mixture was allowed to cool and then was partitioned between EtOAc and aqueous NaHCO3. The EtOAc layer was washed with saturated brine, dried over sodium sulfate, filter, and evaporated to dryness. The crude product was purified by silica gel column chromatography using a gradient of 0-100% EtOAc/hexanes), followed by preparative chiral HPLC using a Chiralpak ADH column and eluting with EtOH/hetpane (1:1) to afford 930 mg of the titled compound.

### (f) 5-[4-(1-{[(3S)-1-(Cyclopropylcarbonyl)-3-pyrrolidinyl]methyl}-6-fluoro-1H-benzimidazol-2-yl)phenyl]-1H-indazole

2-(4-Bromophenyl)-1-{[(3S)-1-(cyclopropylcarbonyl)-3-pyrrolidinyl]methyl}-6-fluoro-1H-benzimidazole (45 mg, 0.102 mmol), 5-(4,4,5,5-tetramethyl-1,3,2-dioxaborolan-2-yl)-1H-indazole (37.2 mg, 0.153 mmol), cesium carbonate (99 mg, 0.305 mmol) and bis(tri-t-butylphosphine)palladium(0) (5.20 mg, 10.17 μmmol) were added to a microwave vial and purged with nitrogen. Dioxane (800 μl) was added followed by water (250 μA), and the reaction mixture heated overnight at 90° C. The reaction mixture was filtered and purified by preparative reverse phase HPLC. The appropriate fractions were combined, the pH neutralized with aqueous NaHCO3 and extracted with EtOAc. The EtOAc extract was dried over sodium sulfate, filtered and evaporated to dryness. The product was further purified by silica gel column chromatography using a gradient of 10-100% EtOAc/hexanes to afford 19.3 mg of the titled compound as a white solid. (LCMS m/z 480.1, M+H).

### Example 181

### 1-{[(3S)-1-(Cyclopropylcarbonyl)-3-pyrrolidinyl]methyl}-6-fluoro-2-[4-(1H-indol-6-yl)phenyl]-1H-benzimidazole

The titled compound was prepared according to the procedure in Example 180f, substituting 6-(4,4,5,5-tetramethyl-1,3,2-dioxaborolan-2-yl)-1H-indole (37.1 mg, 0.153 mmol), to afford 33.1 mg of the expected product. (LCMS m/z 479.1, M+H).

### Example 182

### 6-[4-(1-{[(3S)-1-(Cyclopropylcarbonyl)-3-pyrrolidinyl]methyl}-6-fluoro-1H-benzimidazol-2-yl)phenyl]-1H-indazole

2-(4-Bromophenyl)-1-{[(3S)-1-(cyclopropylcarbonyl)-3-pyrrolidinyl]methyl}-6-fluoro-1H-benzimidazole (45 mg, 0.102 mmol), 6-(4,4,5,5-tetramethyl-1,3,2-dioxaborolan-2-yl)-1H-indazole (37.2 mg, 0.153 mmol), cesium carbonate (99 mg, 0.305 mmol) and bis(tri-t-butylphosphine)palladium(0) (5.20 mg, 10.17 μmmol) were added to a microwave vial and degassed with nitrogen. Dioxane (800 μl) was added followed by water (250 μl) and the reaction mixture was stirred t 90° C. overnight. The reaction mixture was filtered and purifed by preparative reverse phase HPLC. The appropriate fractions were neutralized with aqueous NaHCO3 and extracted with EtOAc. The combined EtOAc extracts were dried over sodium sulfate, filtered and evaporated to afford 27.4 mg of the titled compound as a white solid. (LCMS m/z 480.1, M+H).

### Example 183

### N′-[4′-(1-{[(3S)-1-(cyclopropylcarbonyl)-3-pyrrolidinyl]methyl}-6-fluoro-1H-benzimidazol-2-yl)-3-biphenylyl]-N,N-dimethylsulfamide

The titled compound was prepared according to the procedure in Example 182, substituting N,N-dimethyl-N′-[3-(4,4,5,5-tetramethyl-1,3,2-dioxaborolan-2-yl)phenyl]sulfamide (49.8 mg, 0.153 mmol). The crude product was purified by preparative reverse phase HPLC as in Example 182 followed by silica gel column chromatography using a gradient of 10-100% EtOAc/hexanes to afford 25.7 mg of the titled compound as a white solid. (LCMS m/z 562.1, M+H).

### Example 184

### 1-{[(3S)-1-(Cyclopropylcarbonyl)-3-pyrrolidinyl]methyl}-6-fluoro-2-(4′-fluoro-4-biphenylyl)-1H-benzimidazole

The titled compound was prepared according to the procedure in Example 182, substituting 2-(4-fluorophenyl)-4,4,5,5-tetramethyl-1,3,2-dioxaborolane (33.9 mg, 0.153 mmol), to afford 33.8 mg of the desired product as a white solid. (LCMS m/z 458.2, M+H).

### Example 185

### 1-{[(3S)-1-(Cyclopropylcarbonyl)-3-pyrrolidinyl]methyl}-6-fluoro-2-[4-(1H-indol-5-yl)phenyl]-1H-benzimidazole

The titled compound was prepared according to the procedure in Example 182, substituting 5-(4,4,5,5-tetramethyl-1,3,2-dioxaborolan-2-yl)-1H-indole (37.1 mg, 0.153 mmol), to afford 32.7 mg of the desired product as a white solid. (LCMS m/z 479.1, M+H).

### Example 186

### 2-[4-(1-Benzofuran-5-yl)phenyl]-1-{[(3S)-1-(cyclopropylcarbonyl)-3-pyrrolidinyl]methyl}-6-fluoro-1H-benzimidazole

The titled compound was prepared according to the procedure in Example 182, substituting 5-(4,4,5,5-tetramethyl-1,3,2-dioxaborolan-2-yl)-1-benzofuran (37.2 mg, 0.153 mmol), to afford 31.7 mg of the desired product as a white solid. (LCMS m/z 480.0, M+H).

### Example 187

### 2-[4-(1-Benzofuran-5-yl)phenyl]-1-{[(3S)-1-(cyclopropylcarbonyl)-3-pyrrolidinyl]methyl}-5-fluoro-1H-benzimidazole

### (a) 1,1-Dimethylethyl (3S)-3-{[(4-fluoro-2-nitrophenyl)amino]methyl}-1-pyrrolidinecarboxylate

1-Chloro-4-fluoronitrobenzene (900 mg), 1,1-dimethylethyl (3S)-3-(aminomethyl)-1-pyrrolidinecarboxylate (1.17 g), CsCO₃ (2.20 g), Pd(OAc)₂ (70 mg) and BINAP (280) mg were combined in a microwave vial under nitrogen. Toluene (14 mL) was added and the reaction was heated at 110° C. until the 1-chloro-4-fluoronitrobenzene was consumed by LCMS analysis. The reaction mixture was allowed to cool and was partitioned between EtOAc and saturated brine. The EtOAc layer was dried over sodium sulfate, filtered and evaporated to dryness to afford 1,1-dimethylethyl (3S)-3-{[(4-fluoro-2-nitrophenyl)amino]methyl}-1-pyrrolidinecarboxylate, which was taken on to the next step without further purification.

### (b) (4-Fluoro-2-nitrophenyl)[(3R)-3-pyrrolidinylmethyl]amine hydrochloride

1,1-Dimethylethyl (3S)-3-{[(4-fluoro-2-nitrophenyl)amino]methyl}-1-pyrrolidinecarboxylate (1.7 g) was dissolved in MeOH (20 mL). To this was added 4 N HCl/dioxane (6.26 mL) and the reaction mixture was allowed to stir at RT overnight. The solvent was removed under reduced pressure to afford (4-fluoro-2-nitrophenyl)[(3R)-3-pyrrolidinylmethyl]amine hydrochloride, which was taken on to the next step without further purification.

### (c) {[(3S)-1-(Cyclopropylcarbonyl)-3-pyrrolidinyl]methyl}(4-fluoro-2-nitrophenyl)amine

Crude (4-fluoro-2-nitrophenyl)[(3R)-3-pyrrolidinylmethyl]amine hydrochloride (2.0 g) was suspended in DCM (20 mL) under nitrogen. To this was added DIEA (6.33 mL) and the reaction mixture allowed to stir at RT for 30 min. Cyclopropylcarbonyl chloride (538 uL) was then added slowly and the reaction mixture stirred for 15 min. The reaction mixture was concentrated under vacuum and the residue dissolved in DCM and washed with aqueous NaHCO3. The DCM layer was dried over sodium sulfate, filtered and evaporated to dryness. The crude product was purified by silica gel column chromatography using a gradient of 0-10% IPA/EtOAc to afford {[(3S)-1-(cyclopropylcarbonyl)-3-pyrrolidinyl]methyl}(4-fluoro-2-nitrophenyl)amine.

### (d) N1-{[(3S)-1-(cyclopropylcarbonyl)-3-pyrrolidinyl]methyl}-4-fluoro-1,2-benzenediamine,

{[(3S)-1-(cyclopropylcarbonyl)-3-pyrrolidinyl]methyl}(4-fluoro-2-nitrophenyl)amine (1.8 g) and 10% Pd/C (0.623 g) in EtOH (30 mL) were stirred under an atmosphere of hydrogen overnight. The reaction mixture was filtered and the filtrate evaporated to dryness to afford N1-{[(3S)-1-(cyclopropylcarbonyl)-3-pyrrolidinyl]methyl}-4-fluoro-1,2-benzenediamine, which was used without further purification.

### (e) 2-(4-Bromophenyl)-1-{[(3S)-1-(cyclopropylcarbonyl)-3-pyrrolidinyl]methyl}-5-fluoro-1H-benzimidazole

N1-{[(3S)-1-(cyclopropylcarbonyl)-3-pyrrolidinyl]methyl}-4-fluoro-1,2-benzenediamine (1.1 g) and 4-bromobenzaldehyde (0.881 g) were placed in a microwave vial, which was then purged with nitrogen. To this was added 1-butanol (15 mL) and the reaction was heated at 90° C. in a microwave reactor for 30 min. An aliquot of 10% Pd/C (˜100 mg) was added and the reaction was heated thermally at 100° C. for 1 hr. The reaction mixture was allowed to cool and was filtered and partitioned between EtOAc and aqueous NaHCO3. The EtOAc layer was dried over sodium sulfate, filtered and evaporated to dryness. The crude product was purified by silica gel column chromatography using a gradient of 0-20% IPA/EtOAc, followed by purification again by silica gel column chromatography using a gradient of 0-15% IPA/EtOAc, followed by preparative chiral HPLC purification on a Chiralpak AD-H column using EtOH/heptanes (1:1) as eluant, to afford 390 mg of the titled compound.

### (f) 2-[4-(1-Benzofuran-5-yl)phenyl]-1-{[(3S)-1-(cyclopropylcarbonyl)-3-pyrrolidinyl]methyl}-5-fluoro-1H-benzimidazole

2-(4-bromophenyl)-1-{[(3S)-1-(cyclopropylcarbonyl)-3-pyrrolidinyl]methyl}-5-fluoro-1H-benzimidazole (55 mg, 0.093 mmol), 5-(4,4,5,5-tetramethyl-1,3,2-dioxaborolan-2-yl)-1-benzofuran, cesium carbonate (91 mg, 0.280 mmol and bis(tri-t-butylphosphine)palladium(0) (4.77 mg, 9.33 μmmol) were combined in a microwave vial and degassed with nitrogen. Dioxane (800 μl) was added followed by water (250 μA), and the reaction mixture was heated overnight at 90° C. thermally. The reaction mixture was allowed to cool to RT and was filtered and then purified by preparative reverse phase HPLC. The appropriate fractions were combined, neutralized with aqueous NaHCO3 and extracted with EtOAc. The EtOAc extracts were dried over sodium sulfate, filtered and evaporated to dryness to afford 36.8 mg of the titled compound as a white solid. (LCMS m/z 480.0, M+H).

### Example 188

### 1-{[(3S)-1-(Cyclopropylcarbonyl)-3-pyrrolidinyl]methyl}-5-fluoro-2-[4-(1H-indol-6-yl)phenyl]-1H-benzimidazole

The titled compound was prepared according to the procedure in Example 188f, substituting 6-(4,4,5,5-tetramethyl-1,3,2-dioxaborolan-2-yl)-1H-indole (22.67 mg, 0.093 mmol), to afford 36.5 mg of the desired product as a white solid. (LCMS m/z 479.1, M+H).

### Example 189

### 1-{[(3S)-1-(Cyclopropylcarbonyl)-3-pyrrolidinyl]methyl}-5-fluoro-2-[4-(1H-indol-5-yl)phenyl]-1H-benzimidazole

The titled compound was prepared according to the procedure in Example 188f, substituting 5-(4,4,5,5-tetramethyl-1,3,2-dioxaborolan-2-yl)-1H-indole (22.67 mg, 0.093 mmol). The crude product was purified by preparative reverse phase HPLC as in Example 188f followed by silica gel column chromatography using a gradient of 10-100% EtOAc/hexanes to afford 21.4 mg of the titled compound as a white solid. (LCMS m/z 479.1, M+H).

### Example 190

### 5-[4-(1-{[(3S)-1-(cyclopropylcarbonyl)-3-pyrrolidinyl]methyl}-5-fluoro-1H-benzimidazol-2-yl)phenyl]-1H-indazole

The titled compound was prepared according to the procedure in Example 188f, substituting 5-(4,4,5,5-tetramethyl-1,3,2-dioxaborolan-2-yl)-1H-indazole, to afford 14.5 mg of the desired product as a white solid. (LCMS m/z 480.1, M+H).

### Compounds Exemplified

| | | m/z |
|---|---|---|
| Example | Structure | (M + H) |
| 1 | 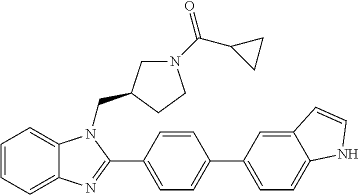 | 461 |
| 2 | 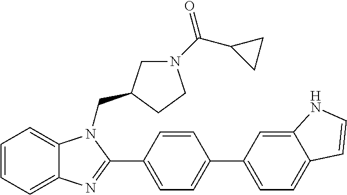 | 461 |
| 3 | 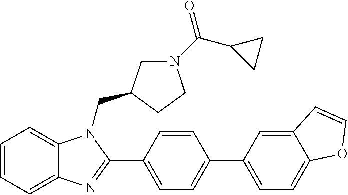 | 462 |
| 4 | 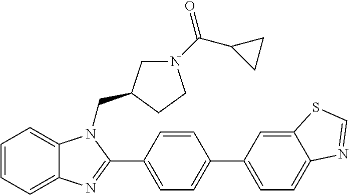 | 479.3 |
| 5 | 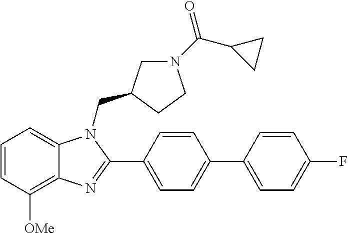 | 470.3 |
| 6 | 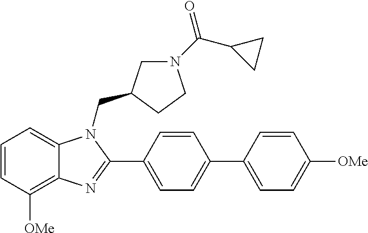 | 482.3 |
| 7 | 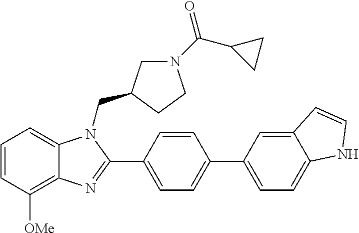 | 491.1 |
| 8 | 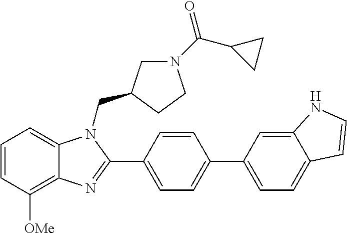 | 491.2 |
| 9 | 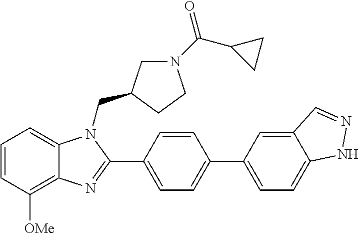 | 492.2 |
| 10 | 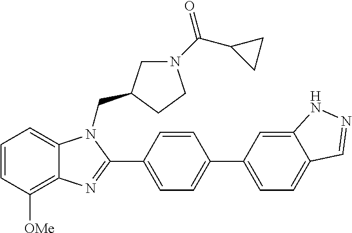 | 492.3 |
| 11 | 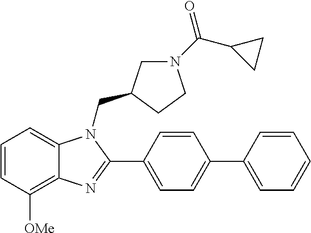 | 452.4 |
| 12 | 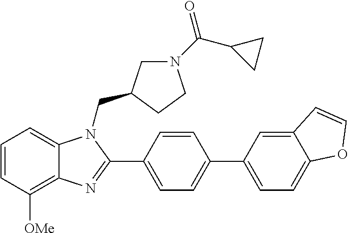 | 492.3 |
| 13 | 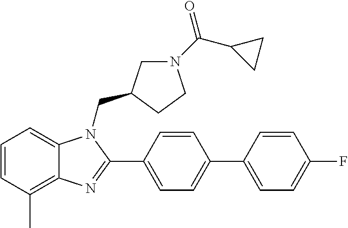 | 454.3 |
| 14 | 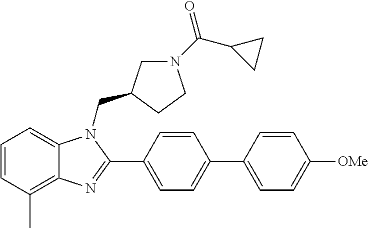 | 466.3 |
| 15 | 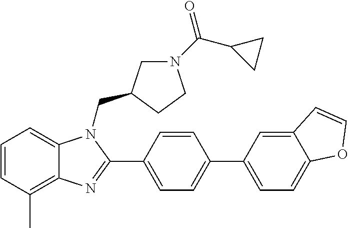 | 476.3 |
| 16 | 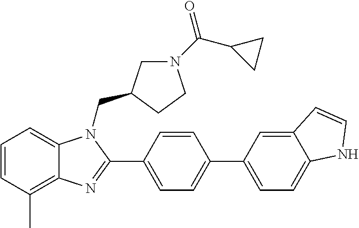 | 475.4 |
| 17 | 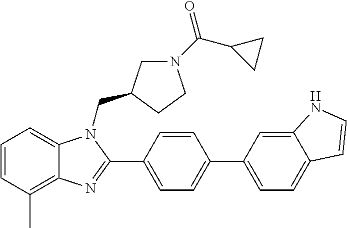 | 475.3 |
| 18 | 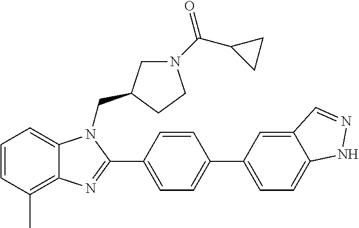 | 476.4 |
| 19 | 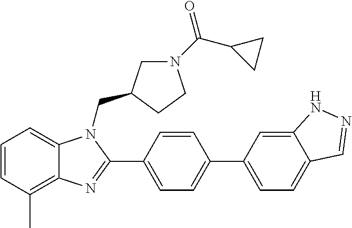 | 476.3 |
| 20 | 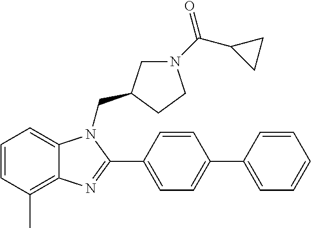 | 436.3 |
| 21 | 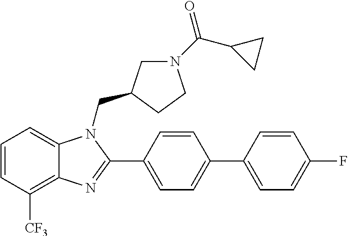 | 508.3 |
| 22 | 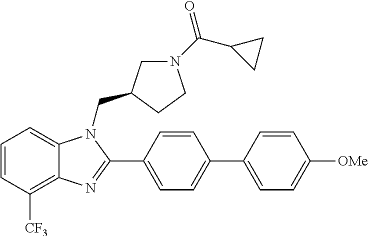 | 520.3 |
| 23 | 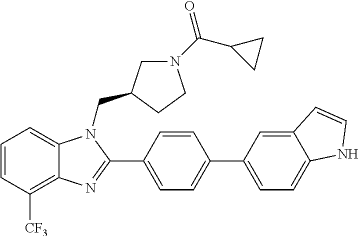 | 529.3 |
| 24 | 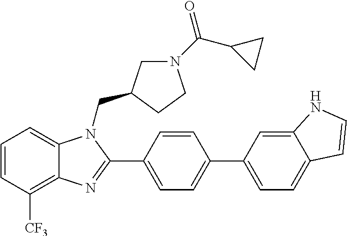 | 529.3 |
| 25 | 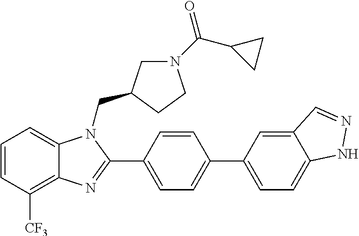 | 530.1 |
| 26 | 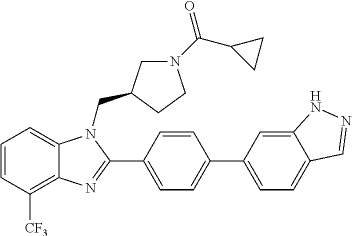 | 530.2 |
| 27 | 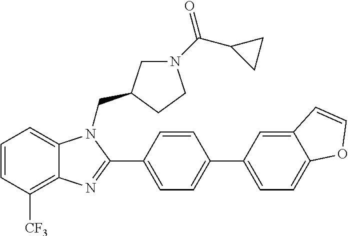 | 530.3 |
| 28 | 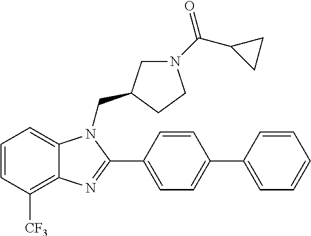 | 490.1 |
| 29 | 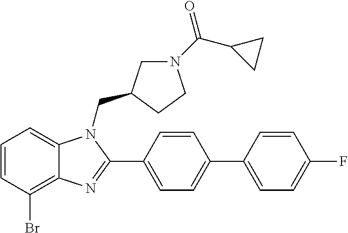 | 518.2 |
| 30 | 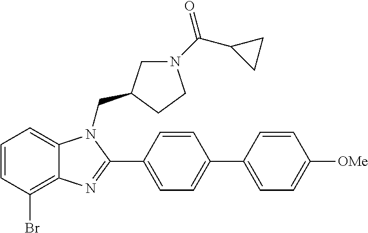 | 530.2 |
| 31 | 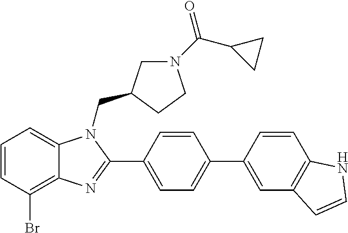 | 539.2 |
| 32 | 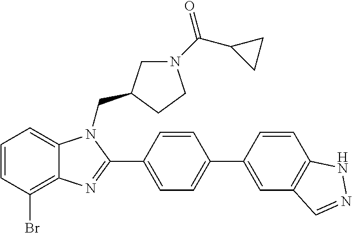 | 540.2 |
| 33 | 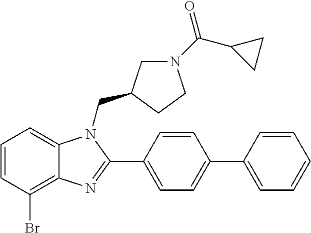 | 500.2 |
| 34 | 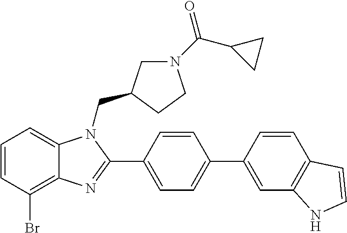 | 539.2 |
| 35 | 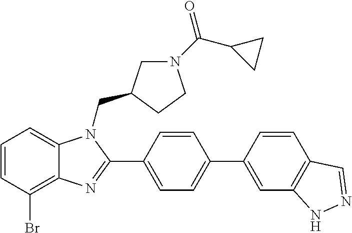 | 540.2 |
| 36 | 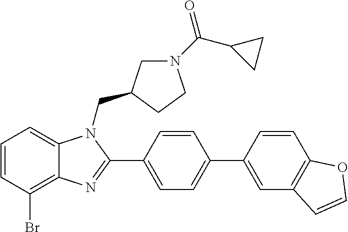 | 540.2 |
| 37 | 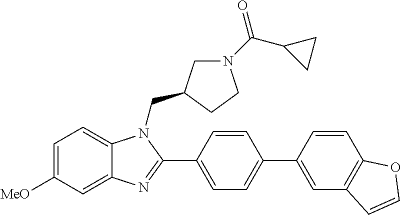 | 492.4 |
| 38 | 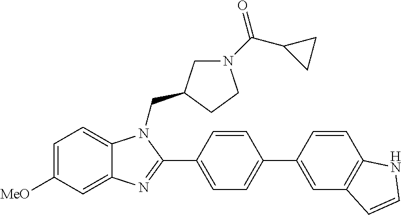 | 491.4 |
| 39 | 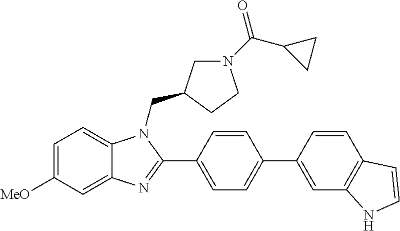 | 491.4 |
| 40 | 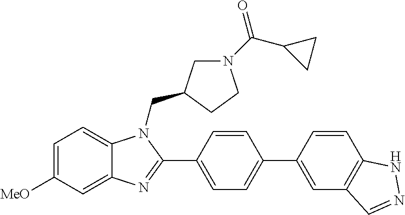 | 492.4 |
| 41 | 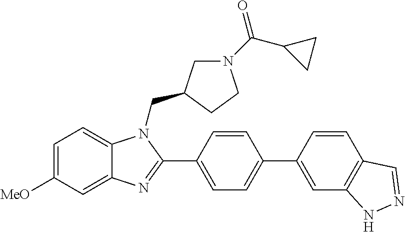 | 492.4 |
| 42 | 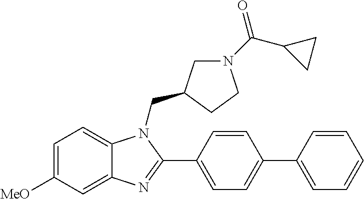 | 452.4 |
| 43 | 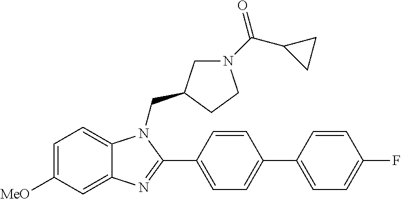 | 470.4 |
| 44 | 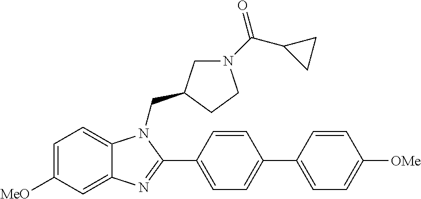 | 482.4 |
| 45 | 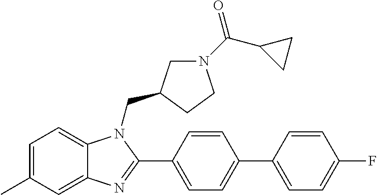 | 454.2 |
| 46 | 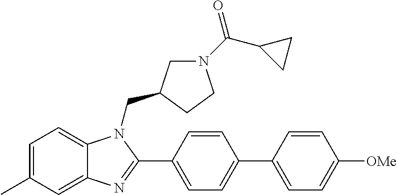 | 466.2 |
| 47 | 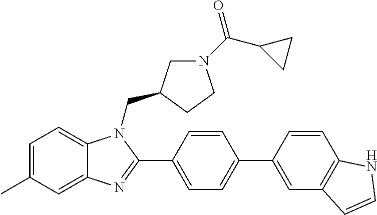 | 475.4 |
| 48 | 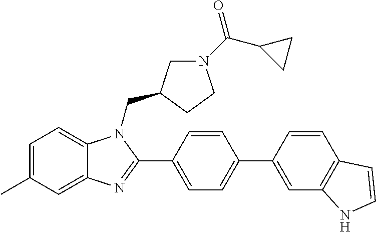 | 475.3 |
| 49 | 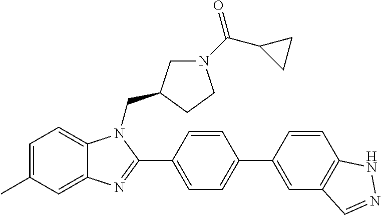 | 476.4 |
| 50 | 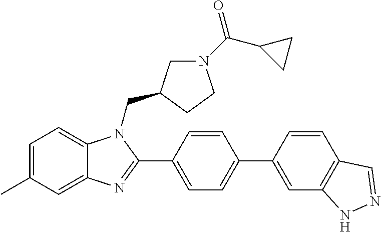 | 476.4 |
| 51 | 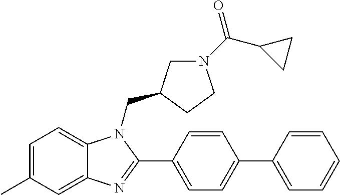 | 436.0 |
| 52 | 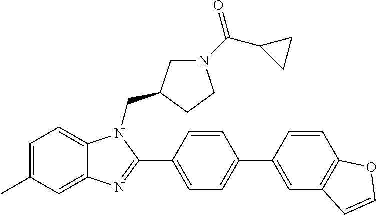 | 476.3 |
| 53 | 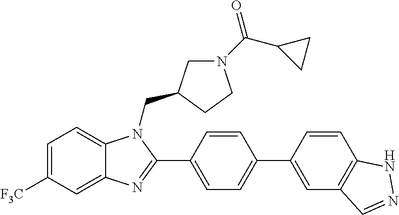 | 530.4 |
| 54 | 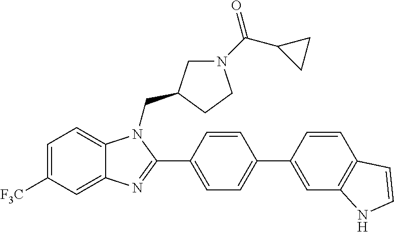 | 529.3 |
| 55 | 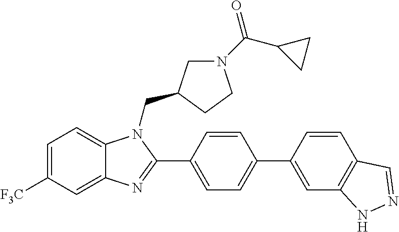 | 530.4 |
| 56 | 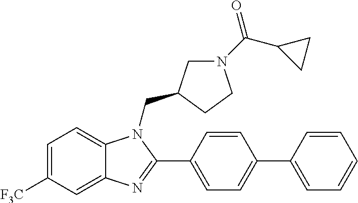 | 490.4 |
| 57 | 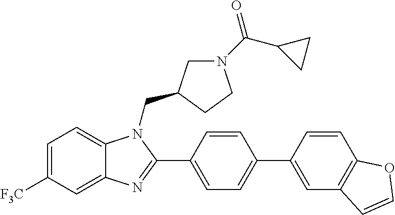 | 530.4 |
| 58 | 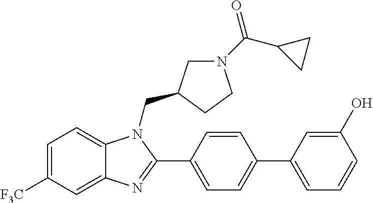 | 506.2 |
| 59 | 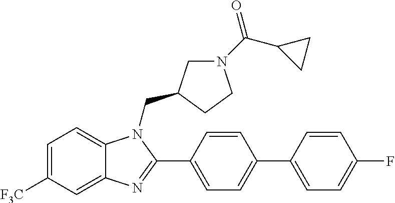 | 508.2 |
| 60 | 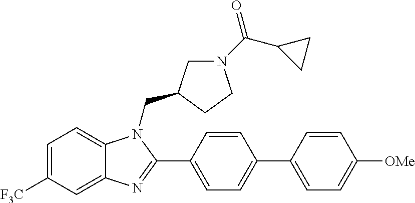 | 520.1 |
| 61 | 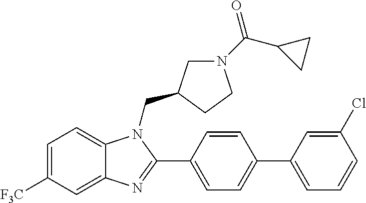 | 524.3 |
| 62 | 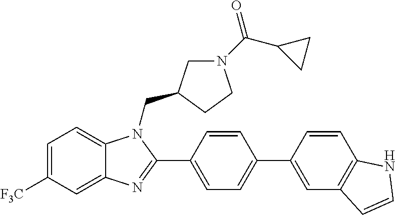 | 529.1 |
| 63 | 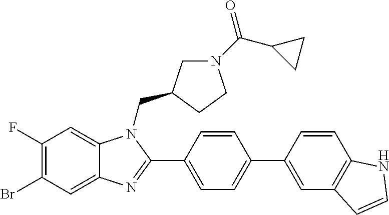 | 557.4 |
| 64 | 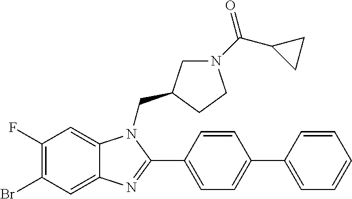 | 518.2 |
| 65 | 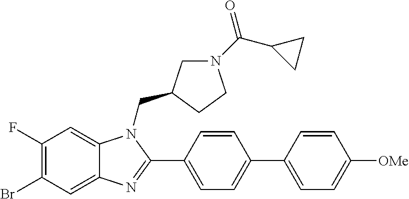 | 548.1 |
| 66 | 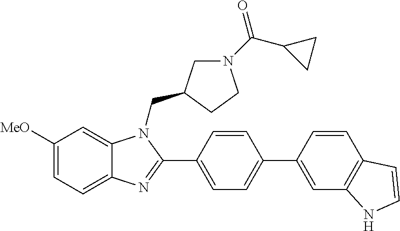 | 491.2 |
| 67 | 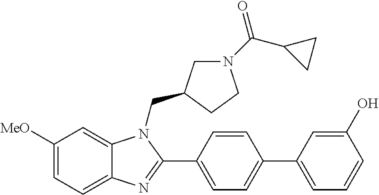 | 468.2 |
| 68 | 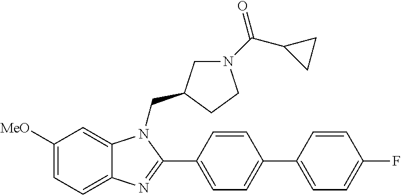 | 470.2 |
| 69 | 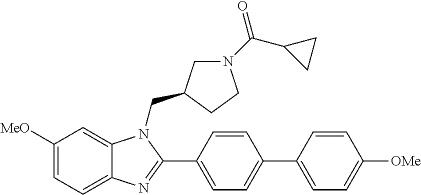 | 482.1 |
| 70 | 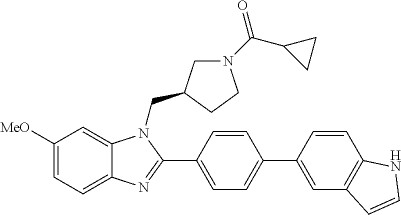 | 491.2 |
| 71 | 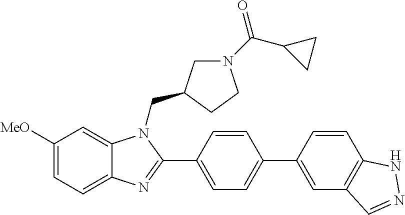 | 492.3 |
| 72 | 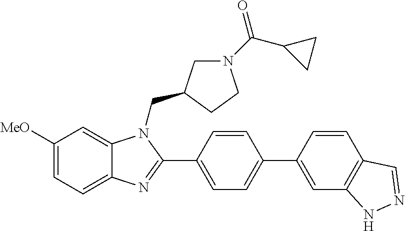 | 492.3 |
| 73 | 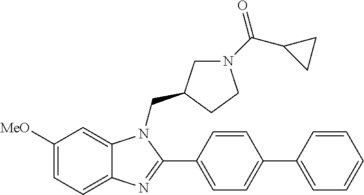 | 452.2 |
| 74 | 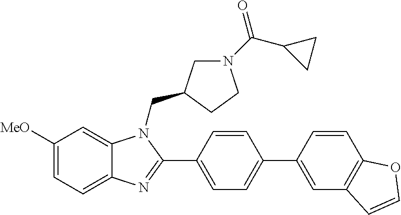 | 492.4 |
| 75 | 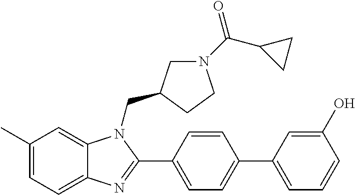 | 452.2 |
| 76 | 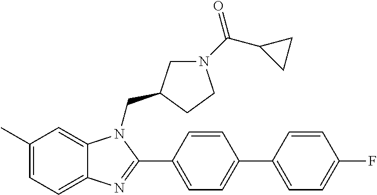 | 454.2 |
| 77 | 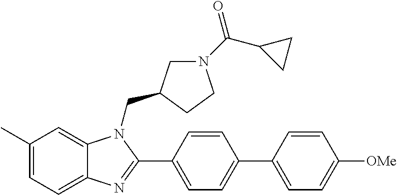 | 466.2 |
| 78 | 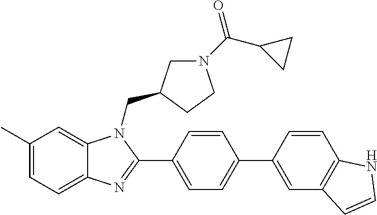 | 475.3 |
| 79 | 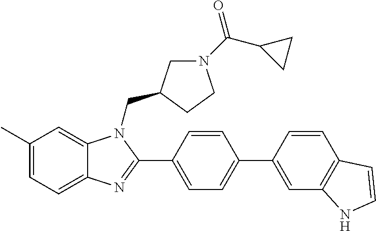 | 475.3 |
| 80 | 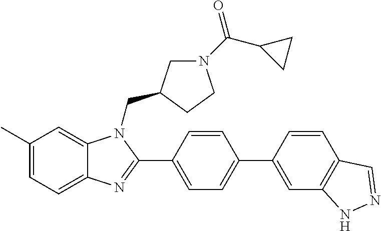 | 476.2 |
| 81 | 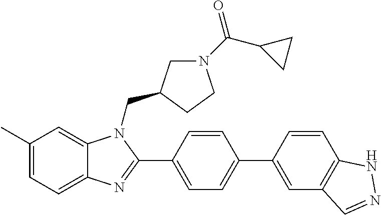 | 476.2 |
| 82 | 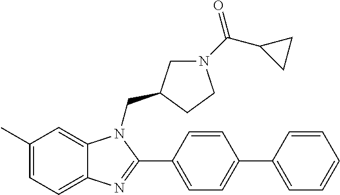 | 436.2 |
| 83 | 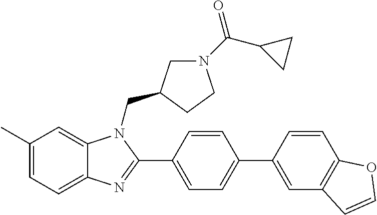 | 476.4 |
| 84 | 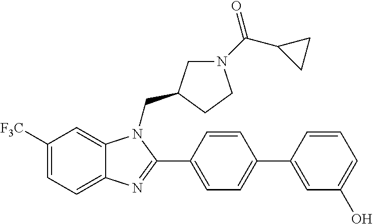 | 506.1 |
| 85 | 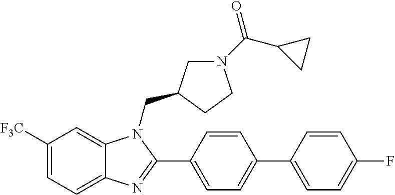 | 508.1 |
| 86 | 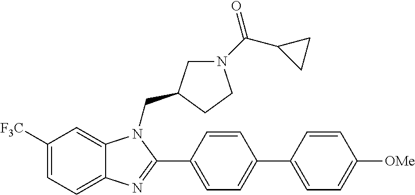 | 520.1 |
| 87 | 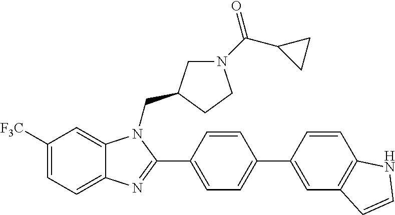 | 529.4 |
| 88 | 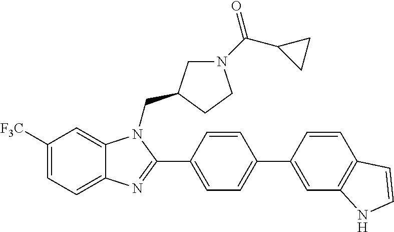 | 529.4 |
| 89 | 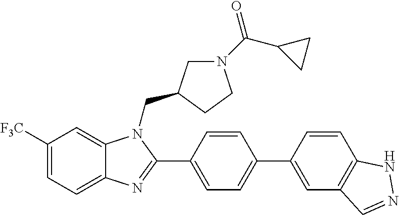 | 530.4 |
| 90 | 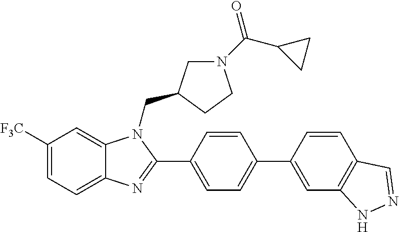 | 530.4 |
| 91 | 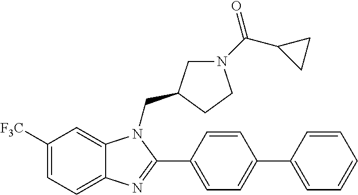 | 490.4 |
| 92 | 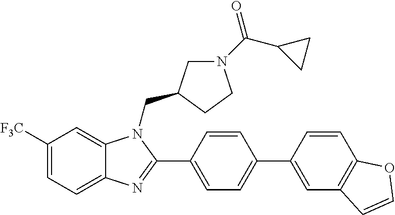 | 530.3 |
| 93 | 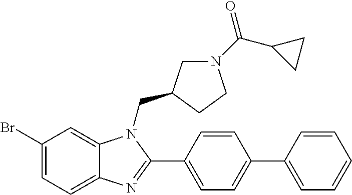 | 500.1 |
| 94 | 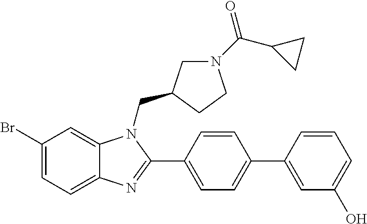 | 516.1 |
| 95 | 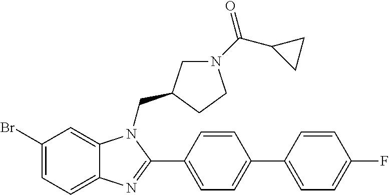 | 518.1 |
| 96 | 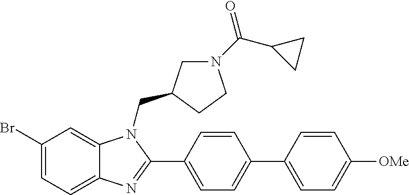 | 530.1 |
| 97 | 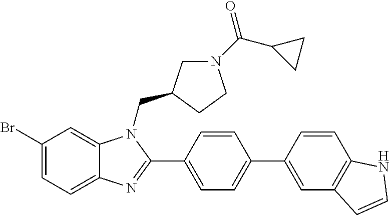 | 539.1 |
| 98 | 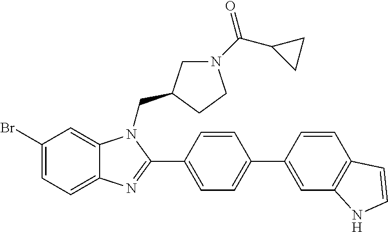 | 539.1 |
| 99 | 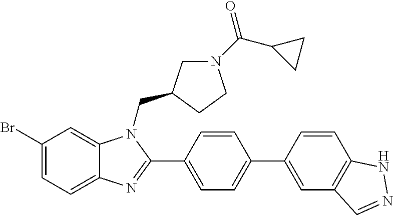 | 540.1 |
| 100 | 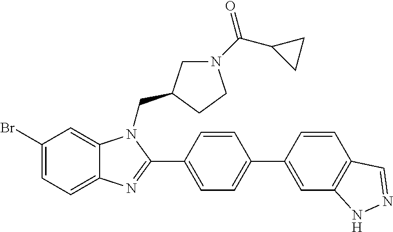 | 540.1 |
| 101 | 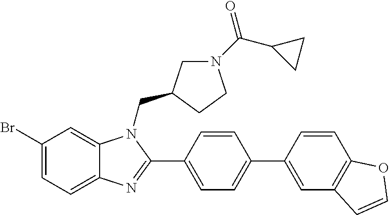 | 540.1 |
| 102 | 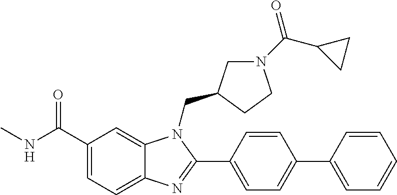 | 479.4 |
| 103 | 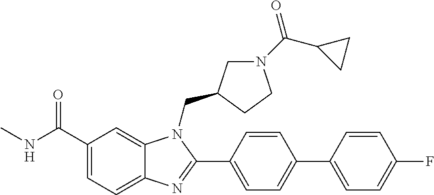 | 497.2 |
| 104 | 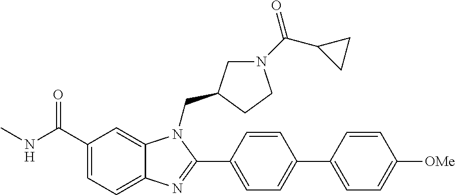 | 509.2 |
| 105 | 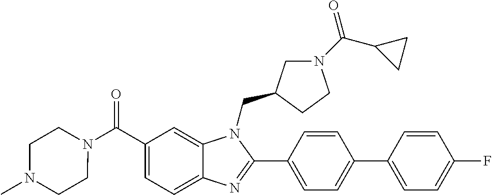 | 566.3 |
| 106 | 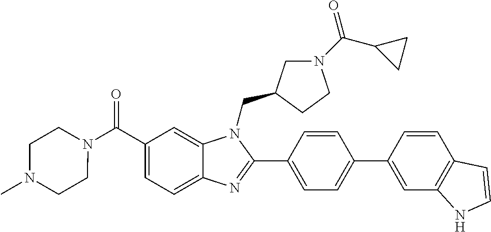 | 587.3 |
| 107 | 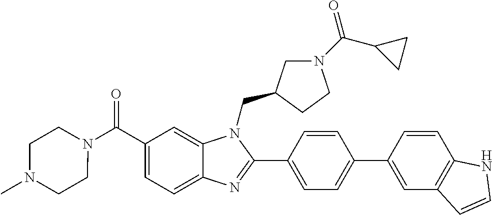 | 587 |
| 108 | 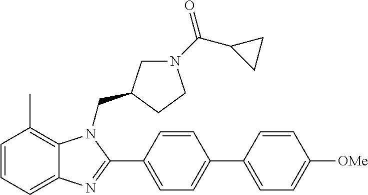 | 466.2 |
| 109 | 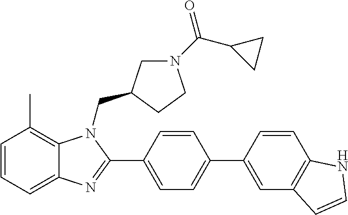 | 475.2 |
| 110 | 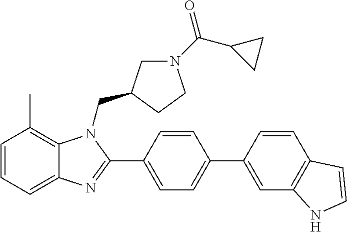 | 475.2 |
| 111 | 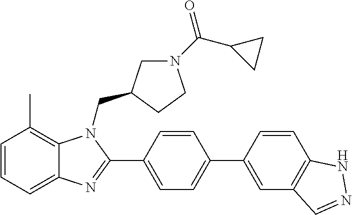 | 476.2 |
| 112 | 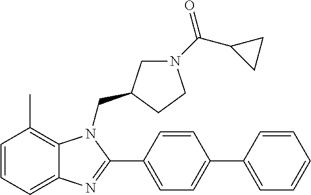 | 436.2 |
| 113 | 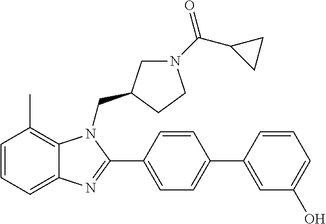 | 452.2 |
| 114 | 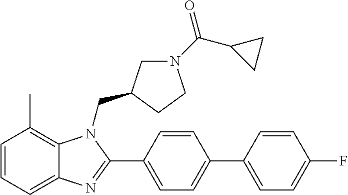 | 454.2 |
| 115 | 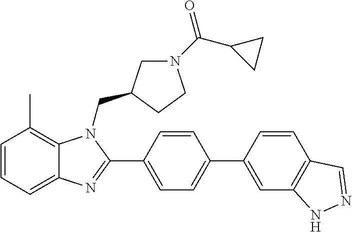 | 476.2 |
| 116 | 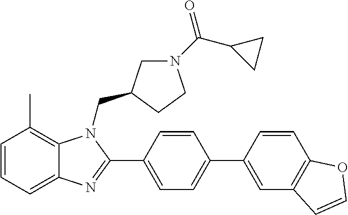 | 476.3 |
| 117 | 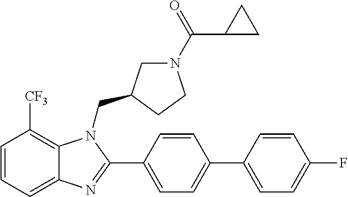 | 508.3 |
| 118 | 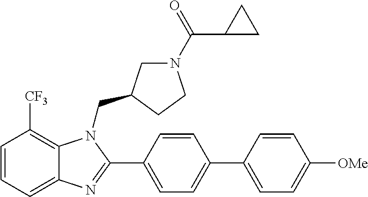 | 520.3 |
| 119 | 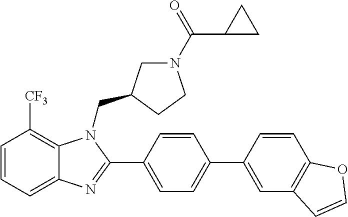 | 530.3 |
| 120 | 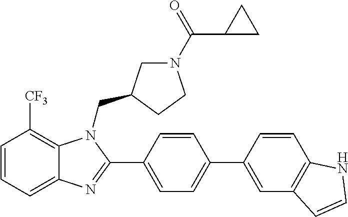 | 529.3 |
| 121 | 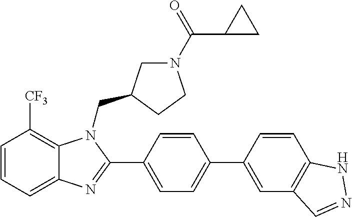 | 530.3 |
| 122 | 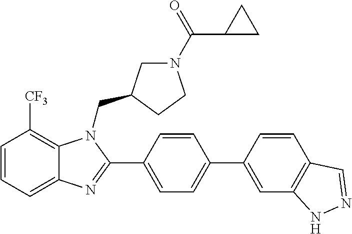 | 530.3 |
| 123 | 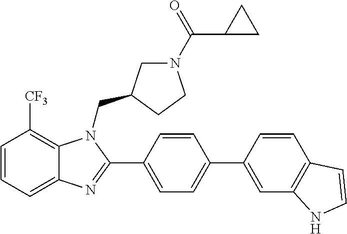 | 529.3 |
| 124 | 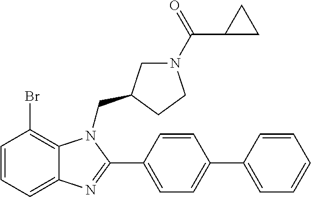 | 500.0 |
| 125 | 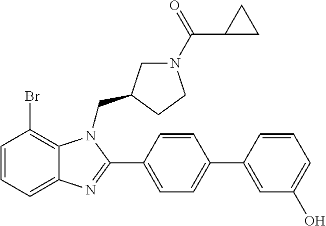 | 516.1 |
| 126 | 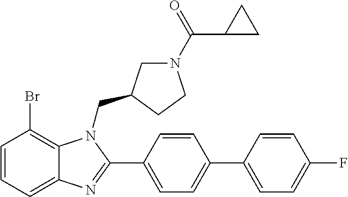 | 518.1 |
| 127 | 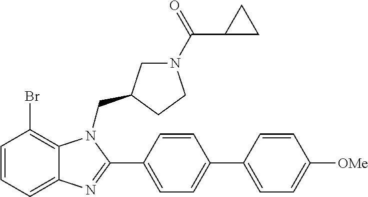 | 530.1 |
| 128 | 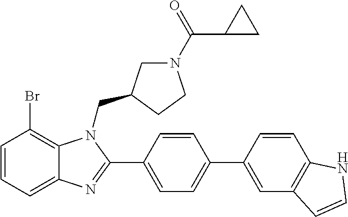 | 539.1 |
| 129 | 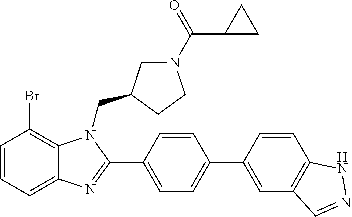 | 540.1 |
| 130 | 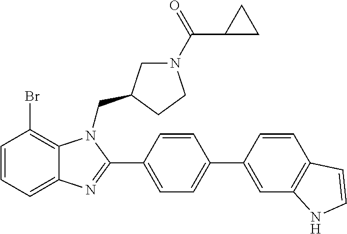 | 539.1 |
| 131 | 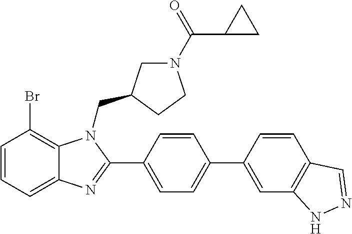 | 540.2 |
| 132 | 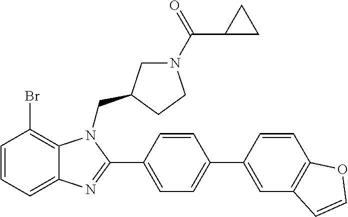 | 540.2 |
| 133 | 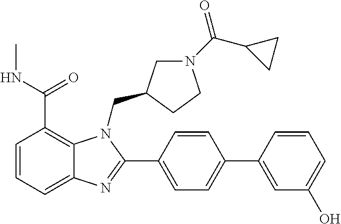 | 495.4 |
| 134 | 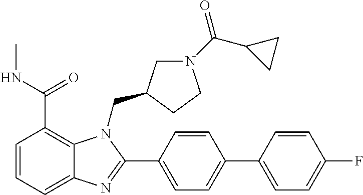 | 497.4 |
| 135 | 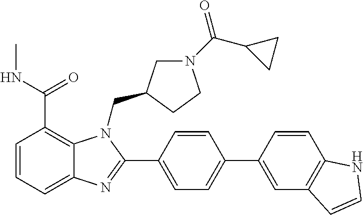 | 518.3 |
| 136 | 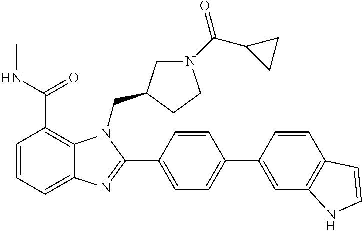 | 518.3 |
| 137 | 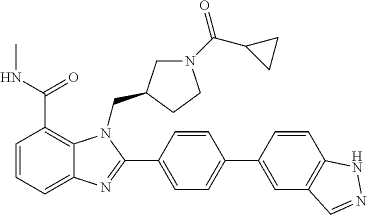 | 519.3 |
| 138 | 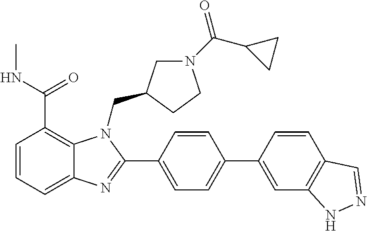 | 519.3 |
| 139 | 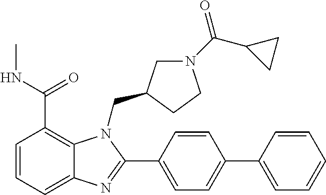 | 479.1 |
| 140 | 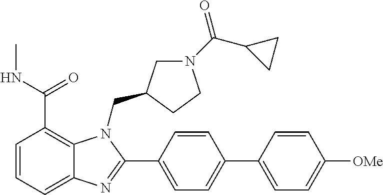 | 509.4 |
| 141 | 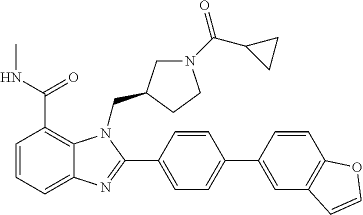 | 519.4 |
| 142 | 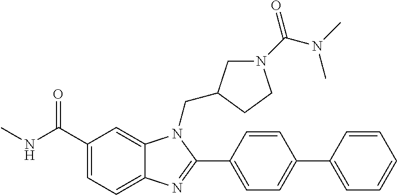 | 482.2 |
| 143 | 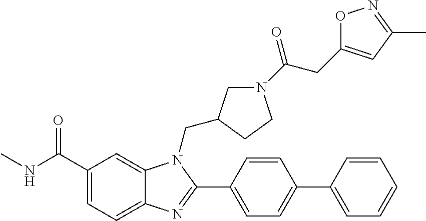 | 534.4 |
| 144 | 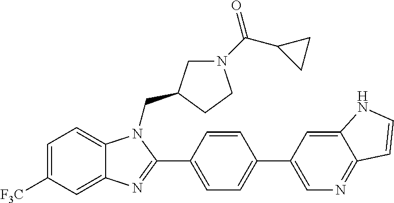 | 530.0 |
| 145 | 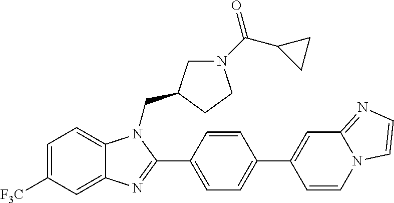 | 529.9 |
| 146 | 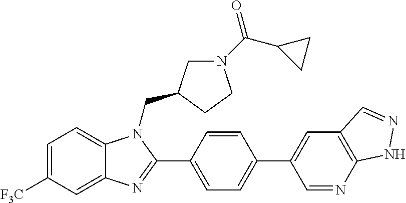 | 530.9 |
| 147 | 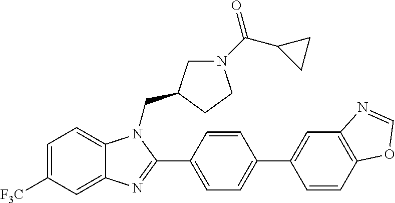 | 530.8 |
| 148 | 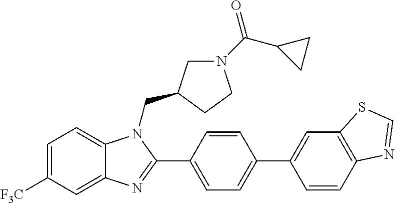 | 547.1 |
| 149 | 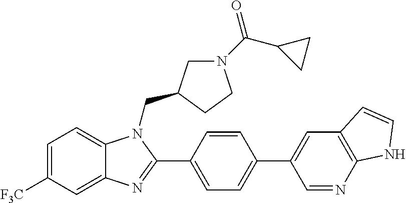 | 529.9 |
| 150 | 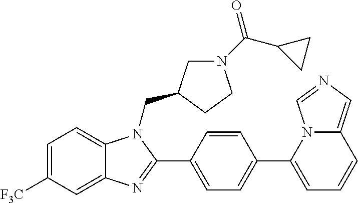 | 529.9 |
| 151 | 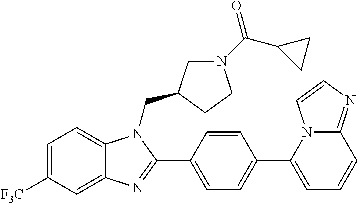 | 530.0 |
| 152 | 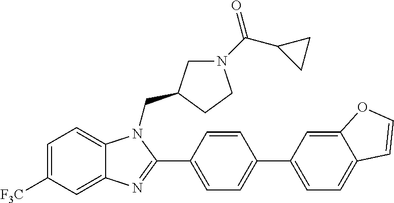 | 530.0 |
| 153 | 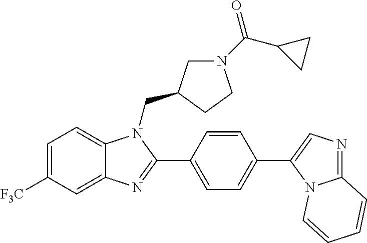 | 530.0 |
| 154 | 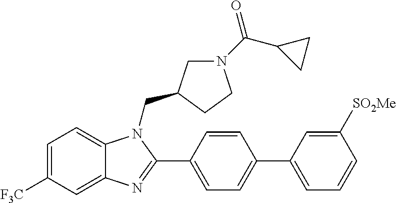 | 568.3 |
| 155 | 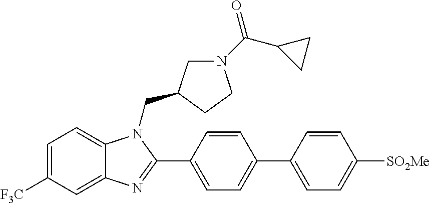 | 568.1 |
| 156 | 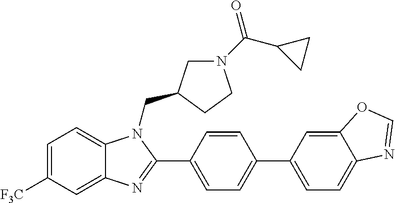 | 531.0 |
| 157 | 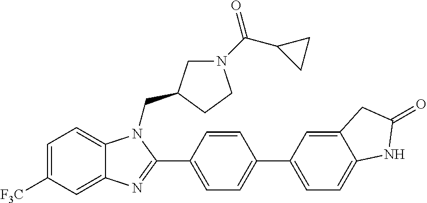 | 545.2 |
| 158 | 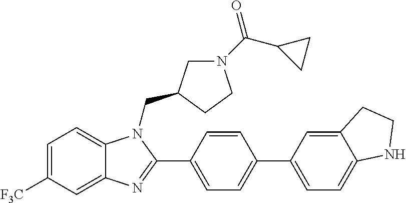 | 530.9 |
| 159 | 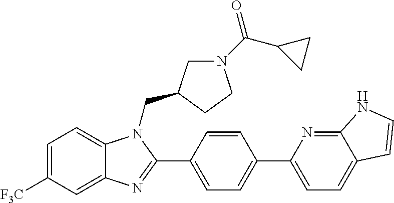 | 530.1 |
| 160 | 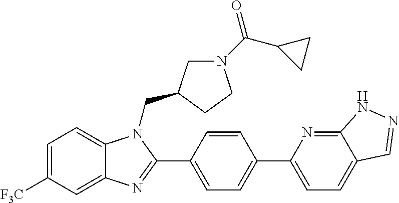 | 530.9 |
| 161 | 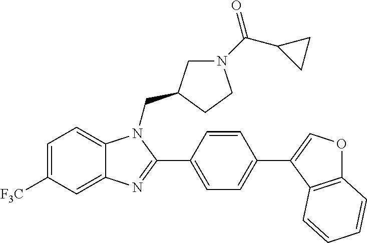 | 530.1 |
| 162 | 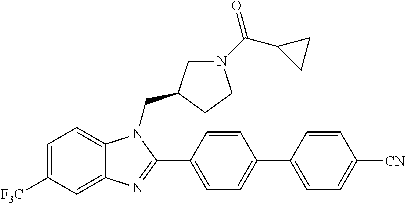 | 515.3 |
| 163 | 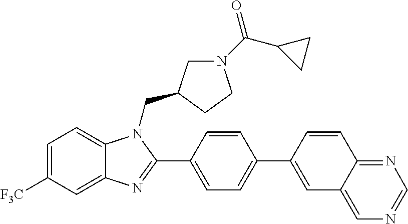 | 542.3 |
| 164 | 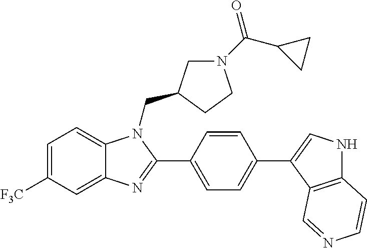 | 529.9 |
| 165 | 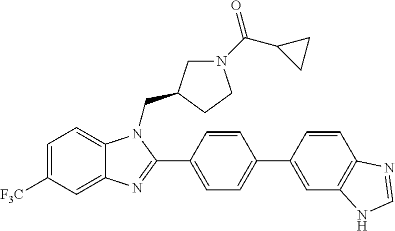 | 530.1 |
| 166 | 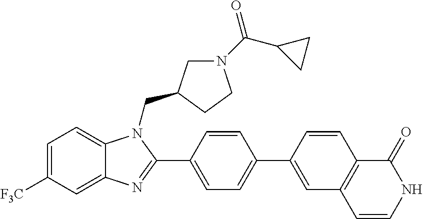 | 556.9 |
| 167 | 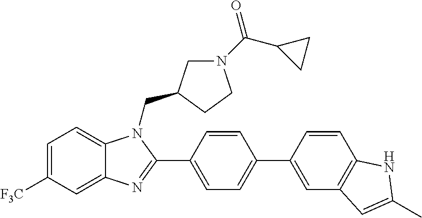 | 543.2 |
| 168 | 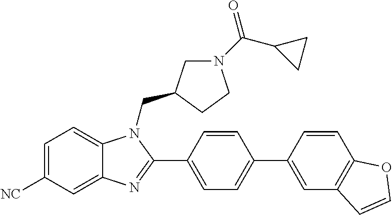 | 487.4 |
| 169 | 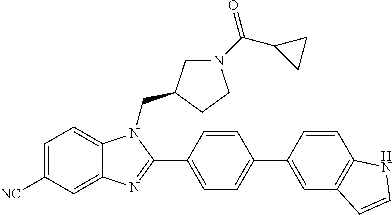 | 486.3 |
| 170 | 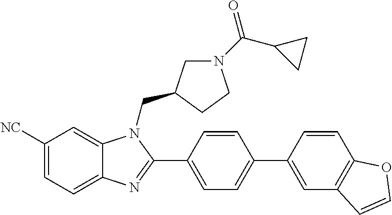 | 487.3 |
| 171 | 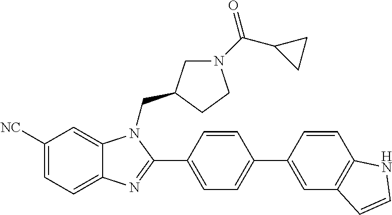 | 486.3 |
| 172 | 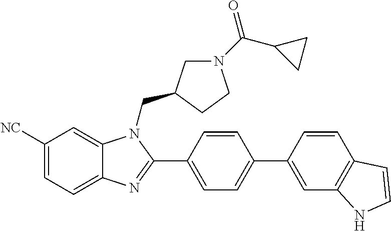 | 486.3 |
| 173 | 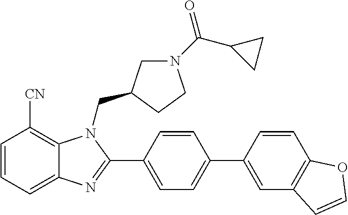 | 487.3 |
| 174 | 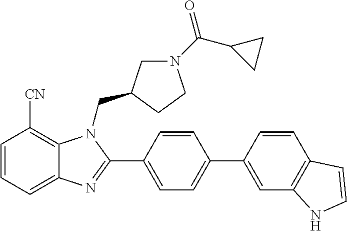 | 486.3 |
| 175 | 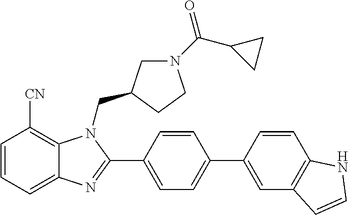 | 486.3 |
| 176 | 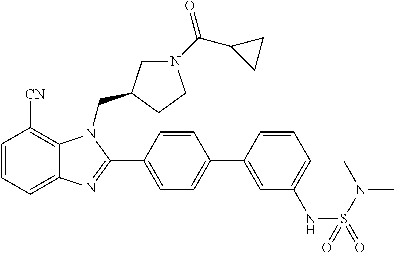 | 569.2 |
| 177 | 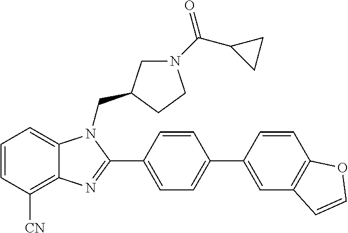 | 487.3 |
| 178 | 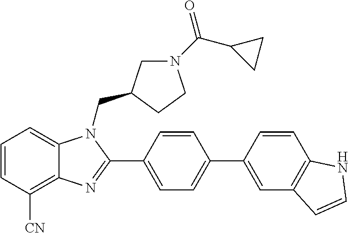 | 486.3 |
| 179 | 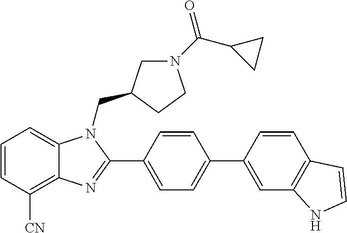 | 486.3 |
| 180 | 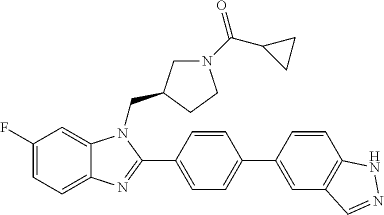 | 480.1 |
| 181 | 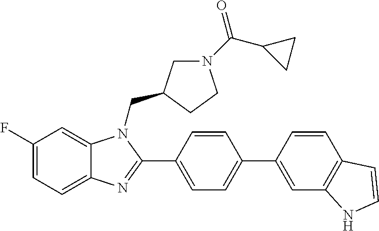 | 479.1 |
| 182 | 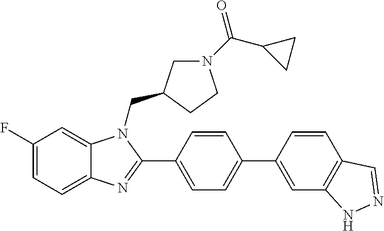 | 480.1 |
| 183 | 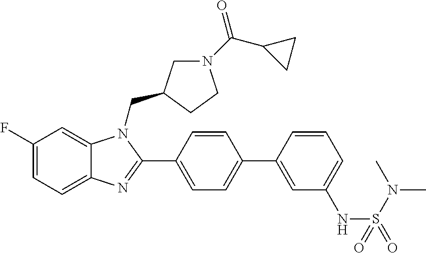 | 562.1 |
| 184 | 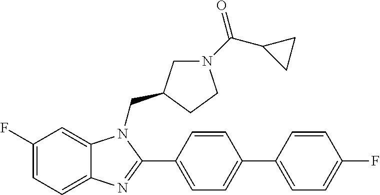 | 458.2 |
| 185 | 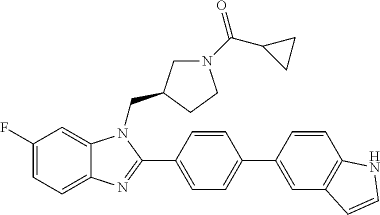 | 479.1 |
| 186 | 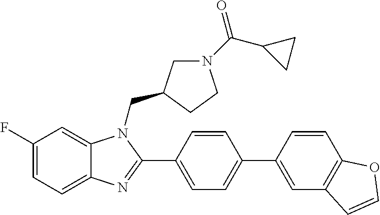 | 480.1 |
| 187 | 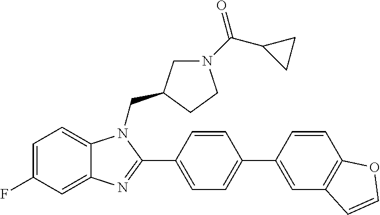 | 480.0 |
| 188 | 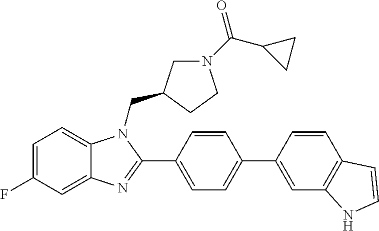 | 479.1 |
| 189 | 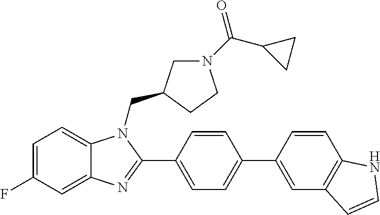 | 479.1 |
| 190 | 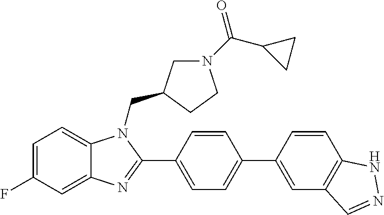 | 480.1 |

### Biological Assays

### FAS Assay

FAS activity was measured through one of the two following assays.

### Assay #1:

Inhibition of FAS activity can be measured based on the detection of residual NADPH substrate after the FAS assay is quenched. This assay is run as a 10 μL endpoint assay in 384-well format, where the reaction contains 20 μM malonyl-CoA, 2 μM acetyl-CoA, 30 μM NADPH and 40 nM FAS in 50 mM sodium phosphate, pH 7.0. The assay is run by sequentially dispensing 5 μl of a malonyl-CoA solution, then enzyme solution (containing the acetyl-CoA, and NADPH) into a black, low volume assay plate (Greiner 784076) pre-dispensed with 100 mL compound solutions in DMSO. The reaction is incubated at ambient temperature for 60 minutes, then quenched with 5 μL of a developing solution composed of 90 μM resazurin, 0.3 IU/ml diaphorase in 50 mM sodium phosphate, pH 7.0. The developed reaction is read on a Molecular Devices Analyst or Acquest (or equivalent) plate reader using a 530 nm excitation wavelength filter, a 580 nm emission filter, and 561 nm dichroic filter. The test compounds are prepared in neat DMSO at a concentration of 10 mM. For inhibition curves, compounds are diluted using a three-fold serial dilution and tested at 11 concentrations (e.g. 25 μM-0.42 nM). Curves are analysed using ActivityBase and XLfit, and results are expressed as pIC50 values.

### Assay #2:

Inhibition of FAS can also be quantified based on the detection of the CoA products with a thio-reactive coumarin dye. This assay is run as a 10 μL endpoint assay in 384-well format, where the reaction contains 20 μM malonyl-CoA, 20 μM acetyl-CoA, 40 μM NADPH and 2 nM FAS in 50 mM sodium phosphate, pH 7.0, and 0.04% Tween-20. The assay is run by adding 5 μL enzyme solution to a black, low volume assay plate (Greiner 784076) pre-dispensed with 100 nl compound solutions in DMSO. After 30 minutes, 50 μL substrate is added, and the reaction incubated at ambient temperature for an additional 60 minutes. The reaction is then quenched with 10 μL of 6M guanidine-HCl containing 50 μM CPM (7-diethylamino-3-(4′-maleimidylphenyl)-4-methylcoumarin (CPM; thio-reactive dye), and incubated for 30 minutes. The plate is read on an Envision (PerkinElmer) or equivalent plate reader using a 380 nm excitation wavelength filter, and a 486 nm emission filter. Data fitting and compound preparations are done as described above.

### Biological Data

Exemplified compounds of the present invention were tested according to the above assays and were found to be inhibitors of FAS. The pIC₅₀ values ranged from about 4.8 to about 8.1.

The compound of Example 4 was tested generally according to the assays described herein and in at least one experimental run exhibited a pIC₅₀ value equal to 7.26.

The compound of Example 24 was tested generally according to the assays described herein and in at least one experimental run exhibited a pIC₅₀ value equal to 7.31.

The compound of Example 33 was tested generally according to the assays described herein and in at least one experimental run exhibited a pIC₅₀ value equal to 6.87.

The compound of Example 49 was tested generally according to the assays described herein and in at least one experimental run exhibited a pIC₅₀ value equal to 7.57.

The compound of Example 62 was tested generally according to the assays described herein and in at least one experimental run exhibited a pIC₅₀ value equal to 7.51.

The compound of Example 66 was tested generally according to the assays described herein and in at least one experimental run exhibited a pIC₅₀ value equal to 7.66.

The compound of Example 83 was tested generally according to the assays described herein and in at least one experimental run exhibited a pIC₅₀ value equal to 7.30.

The compound of Example 88 was tested generally according to the assays described herein and in at least one experimental run exhibited a pIC₅₀ value equal to 7.36

The compound of Example 106 was tested generally according to the assays described herein and in at least one experimental run exhibited a pIC₅₀ value equal to 7.26.

The compound of Example 107 was tested generally according to the assays described herein and in at least one experimental run exhibited a pIC₅₀ value equal to 8.00.

The compound of Example 117 was tested generally according to the assays described herein and in at least one experimental run exhibited a pIC₅₀ value equal to 5.84.

### Lipogenesis Assay

Cultured primary human pre-adipocytes (Zen-Bio, Cat#ASC062801) are plated at confluence (3×10⁴ cells/well) in 96-well plates (Costar, Cat#3598) coated with 0.2% gelatin (Sigma, Cat#G-6650) in DMEM/F12 medium (InvitroGen Cat#11330-032) supplemented with 10% heat inactivated fetal bovine serum (InvitroGen, Cat#16000-044). The following day (day 1) the cell differentiation is induced by replacing the seeding medium with the differentiation medium composed of DMEM/F12 medium supplemented with 10% heat inactivated fetal bovine serum, 200 μM 3-isobutyl-1-methylxanthine (Sigma, Cat#1-5879), 20 nM dexamethasone (Sigma, Cat#D-8893), 20 nM GW1929 (Sigma, Cat#G5668) and 20 nM insulin (InvitroGen, Cat#03-0110SA). On day 7, differentiation medium is replaced by the re-feed medium made of DMEM/F12 supplemented with 10% heat inactivated serum and 20 nM insulin. The appropriate concentration of tested compounds and controls are added into this medium at that time. On day 12, the relative amount of cellular triglyceride is estimated by using a Trinder kit (Sigma, Cat#TR0100). Re-feed medium is aspirated and cells are washed with PBS (InvitroGen, Cat#14190-144) and the assay is performed according the kit manufacturer protocol. Briefly, reconstituted solutions A and B are mixed with 0.01% digitonin (Sigma, Cat#D-5628) prior to performing the assay and added onto the cells; plates are incubated at 37° C. for one hour. The absorbance is read at 540 nm. The data is first normalized using the following equation: 100*((UNK−Control 1)/(Control 2−Control 1)) where Control 1 is the Robust Mean of the 0% response control and Control 2 is the Robust Mean of the 100% response control. When multiple dilutions of compounds are tested, pXC50 are calculated from curves using the 4-parameter curve fitting with the following equation: y=(a−d)/(1+(s/c)̂1))+d and with IRLS (Iterative Re-weighted Least Squares) algorithms to weight outliers (Mosteller, F. & Tukey J. W. (1977) Data Analysis and Regression, pp 353-365, Addison-Wesley).

1. A compound of the Formula (I), 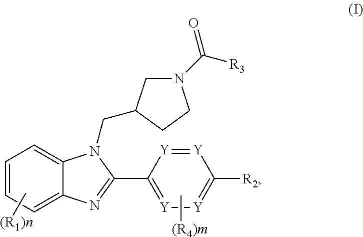
wherein,
each R₁ is independently selected from the group consisting of: halogen, C1-6alkyl, alkoxy, cyano, hydroxyl, amino, substituted amino, alkylsulfonyl, C4-7heterocycloalkyl and —C(O)NRₐR_{b},
in which Rₐ and R_{b} are hydrogen, C1-6alkyl, C3-7cycloalkyl, or together Rₐ and R_{b} form a C4-7heterocycloalkyl;
R₂ is selected from the group consisting of: optionally substituted aryl and heteroaryl, in which adjacent substituents together may form an additional five or six membered ring which contains 0-2 hetero atoms;
R₃ is selected from the group consisting of: amino, alkylamino, dialkylamino, C1-6alkyl, —OC1-6alkyl, and C3-7cycloalkyl;
R₄ is selected from the group consisting of: C1-6alkyl, alkoxy, hydroxyl and halogen;
Y is C or N; and
n is 0-4;
m is 0-4;
or a pharmaceutically acceptable salt thereof;
with the proviso that at least two Y's are C.

2. A compound of claim 1, wherein said compound is represented by Formula (I)(A), as shown below 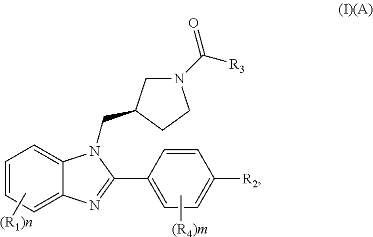
wherein,
each R₁ is independently selected from the group consisting of: C1-6alkyl, cyano, alkoxy, halogen, and —C(O)NRₐR_{b}, in which Rₐ and R_{b} are hydrogen, C1-6alkyl, C3-7cycloalkyl, or together Rₐ and R_{b} form a C4-7heterocycloalkyl;
R₂ is selected from the group consisting of: optionally substituted aryl and heteroaryl, in which adjacent substituents together may form an additional five or six membered ring which contains 0-2 hetero atoms;
R₃ is selected from the group consisting of: C1-6alkyl, —OC1-6alkyl, and C3-7cycloalkyl;
R₄ is selected from the group consisting of: C1-6alkyl, alkoxy, hydroxyl and halogen; and
n is 0-4
m is 0-4;
or a pharmaceutically acceptable salt thereof.

3. A compound of claim 2, wherein R₃ is cyclopropyl.

4. A compound of claim 2, wherein n is 0 and m is 0.

5. A compound of claim 2, wherein R₁ is halogen, cyano, C1-3alkyl, alkoxy, or —C(O)NRₐR_{b} as defined above.

6. A compound of claim 2, wherein R₂ is heteroaryl.

7. A compound of claim 2, wherein R₂ is aryl.

8. A pharmaceutical composition comprising a compound according to claim 1 and a pharmaceutically acceptable carrier.

9. A method of treating cancer which comprises administering to a human in need thereof an effective amount of a compound as described in claim 1.

10. A method of claim 9 wherein the cancer is selected from the group consisting of: brain (gliomas), glioblastomas, leukemias, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, renal, ovarian, pancreatic, prostate, sarcoma, osteosarcoma, giant cell tumor of bone and thyroid.

11. A compound of claim 1, wherein R₃ is cyclopropyl.

12. A compound of claim 1, wherein n is 1 and m is 0.

13. A compound of claim 1, wherein R₁ is halogen, cyano, C1-3alkyl, alkoxy, or —C(O)NRₐR_{b} as defined above.

14. A compound of claim 1, wherein R₂ is heteroaryl.

15. A compound of claim 1, wherein R₂ is aryl.

16. A compound of claim 2, wherein R₂ is an aryl or heteroaryl selected from the group consisting of: indole, phenyl, indazole, benzofuranyl, wherein said aryl or heteroaryl may be substituted by one to three groups selected from: alkyl, halogen, hydroxyl, —SO₂Me and alkoxy.
